SEQUENCE LISTING (38 sequences)

SEQ ID NO: 1 - DNA, 624 bp, Transposon Tn10
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaa

SEQ ID NO: 2 - DNA, 21 bp, Simian virus 40
cctaagaaaaagagaaaagtt

SEQ ID NO: 3 - DNA, 645 bp, Tn10 tet repressor with SV40 NLS
atgcctaagaaaaagagaaaagtttctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaa

SEQ ID NO: 4 - DNA, 550 bp, Artificial Sequence
  Polynucleotide sequence of the tet Triple X operator
aattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactagactctatcagtgatagagtgtatataagactctatcagtgatagagtgaactctatcagtgatagagtt

SEQ ID NO: 5 - DNA, 247 bp, Agrobacterium tumefaciens
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgg

SEQ ID NO: 6 - DNA, 735 bp, Agrobacterium tumefaciens
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggc

SEQ ID NO: 7 - DNA, 108 bp, Artificial Sequence
  Polynucleotide sequence of the TripleX minimal promoter
atctccactgacgtaagggatgacgcacaatcccactagactctatcagtgatagagtgtatataagactctatcagtgatagagtgaactctatcagtgatagagtt

SEQ ID NO: 8 - DNA, 843 bp, A.tumefaciens OCS elements and TripleX minimal promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggcatctccactgacgtaagggatgacgcacaatcccactagactctatcagtgatagagtgtatataagactctatcagtgatagagtgaactctatcagtgatagagtt

SEQ ID NO: 9 - DNA, 1167 bp, Nicotiana tabacum
actattttcagaagaagttcccaatagtagtccaaaatttttgtaacgaagggagcataatagttacatgcaaaggaaaactgccattctttagaggggatgcttgtttaagaacaaaaaatatatcactttcttttgttccaagtcattgcgtatttttttaaaaatatttgttccttcgtatatttcgagcttcaatcactttatggttctttgtattctggctttgctgtaaatcgtagctaaccttcttcctagcagaaattattaatacttgggatatttttttagaatcaagtaaattacatattaccaccacatcgagctgcttttaaattcatattacagccatataggcttgattcattttgcaaaatttccaggatattgacaacgttaacttaataatatcttgaaatattaaagctattatgattaggggtgcaaatggaccgagttggttcggtttatatcaaaatcaaaccaaaccaactatatcggtttggattggttcggttttgccgggttttcagcattttctggttttttttttgttagatgaatattattttaatcttactttgtcaaatttttgataagtaaatatatgtgttagtaaaaattaattttttttacaaacatatgatctattaaaatattcttataggagaattttcttaataacacatgatatttatttattttagtcgtttgactaatttttcgttgatgtacactttcaaagttaaccaaatttagtaattaagtataaaaatcaatatgatacctaaataatgatatgttctatttaattttaaattatcgaaatttcacttcaaattcgaaaaagatatataagaattttgatagattttgacatatgaatatggaagaacaaagagattgacgcattttagtaacacttgataagaaagtgatcgtacaaccaattatttaaagttaataaaaatggagcacttcatatttaacgaaatattacatgccagaagagtcgcaaatatttctagatattttttaaagaaaattctataaaaagtcttaaaggcatatatataaaaactatatatttatattttggtttggttcgaatttgttttactcaataccaaactaaattagaccaaatataattgggatttttaatcg

SEQ ID NO: 10 - DNA, 857 bp, Artificial Sequence
  Polynucleotide sequence of the CtToo 35 Smin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggccgcgtatctccactgacgtaagggatgacgcacaatcccactagactctatcagtgatagagtgtatataagaacctgcaatttggagaggacacgctgcaatttggagaggacacgctgca

SEQ ID NO: 11 - DNA, 1596 bp, Artificial Sequence
  Polynucleotide sequence of the CtTtt 35Smin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggcgatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggccgcgtatctccactgacgtaagggatgacgcacaatcccactagactctatcagtgatagagtgtatataagaacctgcactctatcagtgatagagtctgcactctatcagtgatagagtctgca

SEQ ID NO: 12 - DNA, 884 bp, Artificial Sequence
  Polynucleotide sequence of the CtTttt 35Smin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggccgcgtatctccactgacgtaagggatgacgcacaatcccactagactctatcagtgatagagtgtatataagaacctgcactctatcagtgatagagtctgcactctatcagtgatagagtctgcactctatcagtgatagagtctgca

SEQ ID NO: 13 - DNA, 859 bp, Artificial Sequence
  Polynucleotide sequence of the CtTot 35Smin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggccgcgtatctccactgacgtaagggatgacgcacaatcccactagactctatcagtgatagagtgtatataagaacctgcaatttggagaggacacgctgcactctatcagtgatagagtctgca

SEQ ID NO: 14 - DNA, 857 bp, Artificial Sequence
  Polynucleotide sequence of the CoToo 35Smin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggccgcgtatctccactgacgtaagggatgacgcacaatcccactatccttcgcaagacccttcctctatataagaacctgcaatttggagaggacacgctgcaatttggagaggacacgctgca

SEQ ID NO: 15 - DNA, 861 bp, Artificial Sequence
  Polynucleotide sequence of the CoTtt 35Smin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggccgcgtatctccactgacgtaagggatgacgcacaatcccactatccttcgcaagacccttcctctatataagaacctgcactctatcagtgatagagtctgcactctatcagtgatagagtctgca

SEQ ID NO: 16 - DNA, 859 bp, Artificial Sequence
  Polynucleotide sequence of the CtTto 35Smin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggccgcgtatctccactgacgtaagggatgacgcacaatcccactagactctatcagtgatagagtgtatataagaacctgcactctatcagtgatagagtctgcaatttggagaggacacgctgca

SEQ ID NO: 17 - DNA, 859 bp, Artificial Sequence
  Polynucleotide sequence of the CoTot 35Smin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggccgcgtatctccactgacgtaagggatgacgcacaatcccactatccttcgcaagacccttcctctatataagaacctgcaatttggagaggacacgctgcactctatcagtgatagagtctgca

SEQ ID NO: 18 - DNA, 878 bp, Artificial Sequence
  Polynucleotide sequence of the CoTott 35Smin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggctatctccactgacgtaagggatgacgcacaatcccactatccttcgcaagacccttcctctatataagaacctgcaatttggagaggacacgctgcactctatcagtgatagagtctgcactctatcagtgatagagtctgca

SEQ ID NO: 19 - DNA, 858 bp, Artificial Sequence
  Polynucleotide sequence of the CoTto 35Smin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggccgcgtatctccactgacgtaagggatgacgcacaatcccactatccttcgcaagacccttcctctatataagaacctgcactctatcagtgatagagtctgcaatttggagaggacacgctgc

SEQ ID NO: 20 - DNA, 774 bp, Artificial Sequence
  Polynucleotide sequence of the CTo MASmin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggcaaatggataaatactgcaatttggagaggacacgctgca

SEQ ID NO: 21 - DNA, 792 bp, Artificial Sequence
  Polynucleotide sequence of the tCTo MASmin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggcctctatcagtgatagagtaaatggataaatactgcaatttggagaggacacgctgca

SEQ ID NO: 22 - DNA, 845 bp, Artificial Sequence
  Polynucleotide sequence of the CTTttTTt MASmin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggcaaatggataaatactgcagatataataactctatcactgatagagtgcactctatcagtgatagagttattatatctgcagatataataactctatcactgatagagtgc

SEQ ID NO: 23 - DNA, 802 bp, Artificial Sequence
  Polynucleotide sequence of the tCTtT MASmin promoter
gatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggcctctatcagtgatagagtaaatggataaatactgcactctatcagtgatagagttattatatctgca

SEQ ID NO: 24 - DNA, 23 bp, Artificial Sequence
  tet operator fragment
ctctatcagtgatagagtctgca

SEQ ID NO: 25 - DNA, 23 bp, Artificial Sequence
  tet operator fragment
gactctatcactgatagagtgca

SEQ ID NO: 26 - DNA, 21 bp, Artificial Sequence
  35S fragment
atttggagaggacacgctgca

SEQ ID NO: 27 - DNA, 21 bp, Artificial Sequence
  35S fragment
gcgtgtcctctccaaattgca

SEQ ID NO: 28 - DNA, 31 bp, Artificial Sequence
  tet operator + MAS tata fragment
ctctatcagtgatagagttattatatctgca

SEQ ID NO: 29 - DNA, 31 bp, Artificial Sequence
  tet operator + MAS tata fragment
gatataataactctatcactgatagagtgca

SEQ ID NO: 30 - DNA, 18 bp, Agrobacterium tumefaciens
  MAS caat-tata fragment
aaatggataaatactgca

SEQ ID NO: 31 - DNA, 18 bp, Agrobacterium tumefaciens
  MAS caat-tata fragment
gtatttatccattttgca

SEQ ID NO: 32 - DNA, 36 bp, Artificial Sequence
  tet operator + MAS caat-tata fragment
ctctatcagtgatagagtaaatggataaatactgca

SEQ ID NO: 33 - DNA, 36 bp, Artificial Sequence
  tet operator + MAS caat-tata fragment
gtatttatccatttactctatcactgatagagtgca

SEQ ID NO: 34 - DNA, 22 bp, Artificial Sequence
  primer
tacgcgtatctccactgacgta

SEQ ID NO: 35 - DNA, 20 bp, Artificial Sequence
  primer
cttatatacactctatcact

SEQ ID NO: 36 - DNA, 20 bp, Artificial Sequence
  primer
cttatatagaggaagggtct

SEQ ID NO: 37 - DNA, 445 bp, Agrobacterium tumefaciens
ctgcaggtcaatcccattgcttttgaagcagctcaacattgatctctttctcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgact

SEQ ID NO: 38 - DNA, 106 bp, Agrobacterium tumefaciens
aaatggataaatagccttgcttcctattatatcttcccaaattaccaatacattacactagcatctgaatttcataaccaatctcgatacaccaaatcgactctag

(19) United States
(12) Patent Application Publication
(10) Pub. No.: US 2005/0034187 A1
(43) Pub. Date: Feb. 10, 2005

(54) Modified tet-inducible system for regulation of gene expression in plants

(76) Inventors: Andrei Golovko, Bristol, PA (US); Gerald Hall Jr, Morrisville, PA (US)

Correspondence Address: KEIL & WEINKAUF, 1350 CONNECTICUT AVENUE, N.W., WASHINGTON, DC, 20036, US

(21) Appl. No.: 10/363,657

(22) PCT Filed: Sep. 7, 2001
(86) PCT No.: PCT/EP01/10315

Related U.S. Application Data
(60) Provisional application No. 60/231,522, filed on Sep. 9, 2000.

(51) Int. Cl. C12N 15/82; C12N 15/85

(52) U.S. Cl. 800/278; 435/320.1; 435/252.3

(57) ABSTRACT

The present invention relates to modified tetracycline-inducible cassettes for controlling gene expression in organisms, particularly plants. Specifically, the invention provides novel tetracycline repressor and operator cassettes. The invention preferably provides a tetracycline-inducible expression cassette comprising both the tetracycline repressor and operator cassettes of the present invention wherein the repressor and operator cassettes are located on a single plasmid and/or vector. Also provided is a method of producing herbicide resistant plants using the modified tetracycline inducible cassettes of the present invention to control the expression of a herbicide resistance gene. Moreover, a method for identifying novel tetracycline analogs and/or functional equivalents using the modified tetracycline inducible cassettes of the present invention is also presented.

### FIELD OF THE INVENTION

The present invention relates to modified tetracycline-inducible cassettes for controlling gene expression in organisms, particularly plants. Specifically, the invention provides novel tetracycline repressor and operator cassettes. The invention preferably provides a tetracycline-inducible expression cassette comprising both the tetracycline repressor and operator cassettes of the present invention wherein the repressor and operator cassettes are located on a single plasmid and/or vector. Also provided is a method of producing herbicide resistant plants using the modified tetracycline inducible cassettes of the present invention to control the expression of a herbicide resistance gene. Moreover, a method for identifying novel tetracycline analogs and/or functional equivalents using the modified tetracycline inducible cassettes of the present invention is also presented.

### BACKGROUND OF THE INVENTION

The ability to reversibly turn genes on and off has great utility for the analysis of gene expression and function, particularly for those genes whose products are toxic to the cell. A well characterized control mechanism in prokaryotes involves repressor proteins binding to operator DNA to prevent transcription initiation (Wray and Reznikoff, 1983), and regulated systems have been developed for controlling the expression, both in animals (Wirtz and Clayton, 1995; Deuschle et al, 1995; Furth et al, 1994; Gossen and Bujard, 1992; Gossen et al, 1995) and plants (Wilde et al, 1992; Gatz et al, 1992; Roder et al, 1994; Ulmasov et al, 1997).

Two major systems have been successfully exploited for regulation of plant gene expression during the past decade: the lac (Ulmasov et al, 1997; Wilde et al, 1992) and tet (Wilde et al, 1992; Gatz et al, 1992; Roder et al, 1994; Ulmasov et al, 1997) operator-repressor systems. Both systems are repressor/operator based-systems and derive key elements from their corresponding prokaryotic operon—the E. coli lactose operon for lac, and the transposon Tn10 tetracycline operon for tet. Generally, these systems control the activity of a promoter by placing operator sequences near the transcriptional start site of a gene such that gene expression from the operon is inhibited upon the binding of the repressor protein to its cognate operator sequence. However, in the presence of an inducing agent, the binding of the repressor to its operator is inhibited—thus activating the promoter and enabling gene expression. In the lac system, isopropyl-B-D-thiogalactopyranoside (IPTG) is the standard inducing agent, while tetracycline, and/or its analog doxycyline, is the standard inducing agent for the tet system.

Although the lac repressor has been extensively characterized, there are several advantages to using a tet repressor based system. For example, despite the fact the lac repressor has a high association constant for its operator, and the fact that isopropyl b-d-thiogalactoside is able to reduce the affinity of repressor for the operator by 300-fold (Barkley and Bourgeois, 1980), a maximum of only 30-fold repression has been documented using the lactose repressor (Ulmasov et al, 1997). However, a 500-fold repression level has been documented using tet-based system (Gatz et al, 1992).

Another advantage concerns the toxicity of the inducing agent. The level of IPTG required to induce a lac repressor system is sufficiently high to be cytotoxic to cells. However, the level of tetracycline, or an analog, required to induce expression in a tet-based system is significantly lower (Gossen, M., et al., Curr. Opin. Biotech., 5:516-520 (1994)).

Tetracycline is the parent compound of a widely used class of antibiotics. Many chemical analogs of tetracycline have been synthesized and studied for their antibacterial effects (Rogalski, 1985). Some of them have markedly different affinities for tet Repressor (Degenkolb et al., 1991) and are up to 100-fold more efficient inducers than tetracycline. Such derivatives have been given a thermodynamic description of induction (Lederer et al, 1996) and used for tet Repressor-regulated expression in eukaryotic systems (Gossen et al., 1995).

As inferred above, the regulated expression of the Tn10-operon is mediated by the binding of the tet Repressor to its operator sequences (Beck et al., 1982, and Wray and Reznikoff, 1983). The high specificity of tetracycline for the tet operator, the high efficiency of inducibility, the low toxicity of the inducer, as well as the ability of tetracycline to easily permeate most cells, are the basis for the application of the tet system in somatic gene regulation in eukaryotic cells: most frequently in animals (Wirtz and Clayton. 1995; Gossen et al, 1995), humans (Deuschle et al, 1995; Furth et al, 1994; Gossen and Bujard, 1992; Gossen et al, 1995), and to a lesser extent, in plant cell cultures (Wilde et al, 1992; Gatz et al, 1992; Roder et al, 1994; Ulmasov et al, 1997).

A number of variations of tetracycline operator/repressor systems have been devised. For example, Gossen and Bujard describe a tetracycline based operator/repressor system which is based upon converting the tet repressor to an activator by constructing fusion proteins between transcriptional transactivation domains (e.g., the transactivator of herpes simplex virus, VP16) and the tet repressor (PNAS, 89:5547-5551, (1992)). In this example, the effector, tetracycline, inactivates the transactivator and thereby inhibits transcription from a minimal promoter that functions solely upon binding of the tet repressor/VP16 fusion protein (tTA) to several tet operator sequences located approximately 70 bp upstream from the transcriptional start site. In the absence of tetracycline, the tTA protein binds to the operator sequences—thus leading to subsequent transcriptional activation. This system has been applied to plants (Weinmann, P., et al., Plant J., 5:559-569, (1994)), rat hearts (Fishman, G I., et al., J. Clin. Invest., 93:1864-1868, (1994)), and more recently in mice (Furth, P A., et al., PNAS, (1994)). However, further characterization of this system determined the chimeric tTA fusion protein was toxic to cells at levels required for efficient gene regulation (Bohl, D., et al., Nat Med, 3:299-305, (1996)).

A central problem with tetracycline inducible promoters, in general, concerns the fairly high level of gene expression observed during non-induced (i.e., repressed) conditions (e.g., in the presence of the inducing agent for tet repressor/operator systems, or in the absence of the inducing agent for tet repressor/transactivator systems). Such “leaky” expression is undesirable in most instances, since it restricts the application of inducible tet promoters largely to non-toxic proteins, or to proteins having low biological activity.

In addition, the majority of the tet inducible promoters described to date have been two plasmid systems where one plasmid contains the gene of interest operably linked to a promoter under the control of tet operator sequences, and the other plasmid contains a constitutive promoter for driving the tet repressor. The application of two plasmid systems to plants, and/or other organisms, poses numerous obstacles, in general. First, both plasmids require separate selection genes to avoid plasmid competition. In the absence of other factors, two plasmids having the same selection genes could compete with each other within the plant cell, resulting in a significantly lower level of one plasmid, compared to the other. This problem is further compounded by the relative scarcity of appropriate selection genes for use in plants.

Secondly, the probability of transfecting both plasmids equally in all cells is greatly diminished. The techniques currently used for transfecting plant cells are highly inefficient and typically result in less than 1% positive transfectants per transformation. Thirdly, the copy number of either plasmid may not be equal in all cell types. For example, some cell types may result in very high copy numbers of one plasmid, and very low levels for the other plasmid. The copy number of the plasmid could have profound effects on the overall level of gene expression, particularly in those instances where the plasmid containing the tet repressor is very low in copy number. In the latter example, the absence of sufficient tet repressor to occupy the tet operator sites could lead to very high levels of “leaky” expression of the gene of interest. It could also greatly diminish the ability of the system to effectively control gene expression. Lastly, random chromosomal integration of both plasmids would also pose significant obstacles since it would complicate the process of obtaining homogenous populations of transgenic plants, for example, in future generations.

Therefore, there is a need in the art for an inducible promoter system that has very tight levels of gene expression (i.e., low leaky expression), that is capable of inducing high levels of gene expression during periods of induction, that utilizes a strong and/or tissue specific promoter, and that is based upon a single plasmid system. Such a system would preferably be applicable to regulating plant gene expression, though could also be applied to regulating genes in other organisms, such as mammals, bacteria, and yeast, for example. In addition, there is also a need in the art to identify additional tetracycline analogs, and/or their equivalents.

The modified tetracycline inducible cassettes of the invention represent a significant advance with respect to the tetracycline-inducible promoters currently available in the art. As discussed supra, the tetracycline inducible promoter cassettes known in the art are prone to high levels of leaky expression—significantly limiting their application. The novel tetracycline repressor cassettes of the present invention alleviate this problem by maximizing the occupancy of the tetracycline operators and minimizing leaky expression of downstream genes through a variety of methods. First, the present invention increases the concentration of tetracycline repressor, albeit indirectly, through the addition of a nuclear localization signal operably linked to the tet repressor coding region. Secondly, the novel tetracycline repressor cassettes of the present invention comprise at least one or more enhancer sequences upstream of the promoter driving the tet repressor (for example, the OCS, (OCS)₃, and MAR elements), and the application of promoters (e.g., which includes the 35s promoter, the MAS promoter, derivations of the 35s and/or MAS promoters, in addition to, other promoters), to drive the tet repressor expression. The addition of the nuclear localization signal to the tet repressor increases the effective concentration of the tet repressor by directing the localization of the protein to the nucleus of the cell. In addition, the enhancer sequences, and promoters, directly result in an increased cellular concentration of tet repressor through increased expression.

A tetracycline regulated expression system specifically designed for transgenic plants has been described (Gatz et al, 1992). The cassette was made in which three tet operators were introduced into the vicinity of the tata box of the cauliflower mosaic virus (CaMV) 35S promoter. When stably integrated into the genome of a tet Repressor-positive plant, the activity of the promoter was reduced up to 500-fold, owing to steric interference of the tet Repressor with the transcription initiation complex. This effect was supported by the high amount of repressor monomers (600 000 per cell in plants exhibiting the highest level of expression) that, in turn, provided fractional saturation of operator sites by the repressor of 0.9999 (Gatz et al, 1991). Addition of the tetracycline inducer, which prevents the repressor from binding to its operator sequences, lead to full derepression of the promoter, and the subsequent activation of gene expression (Gatz et al, 1992).

Although the 35S promoter worked in the context of a tetracycline inducible system, it was not necessarily the best promoter for a variety of reasons. First, the 35S promoter is constitutively active in most plant cell types. Such. global activation may be desirable in some instances, such as in expressing a gene to confer resistance to a plant pathogen, for example. However, using 35S to drive.the expression of a gene useful for promoting fruit ripening would not be desirable if expressed in apical meristem tissue, for example, since it could lead to abscission, and/or necrosis, of such tissue. Therefore, the ability to induce expression of a gene in a particular cell or tissue cell type would be more advantageous.

Secondly, the 35S promoter is not a strong promoter. One of the goals of using an inducible promoter system is to drive the expression of a gene, on demand, and in sufficient amounts to observe the desired trait. However, if the promoter driving the expression is not strong, then it may require increased levels of inducing agent to observe the desired affect—levels that may be toxic to the cells. Additionally, using a modest promoter may take significantly longer to observe the desired trait since it would take more time for the plant or cell to accumulate the gene of interest to a level where it would have a phenotypic effect.

The addition of a nuclear localization signal to the coding region of the tet repressor has been described previously in tet inducible systems in animals and retroviruses (International Publication No: WO 94/04672). However, the addition of a nuclear localization signal to the tet repressor for use in modulating gene expression in a plant has not been observed prior to the present invention. More recent applications of a nuclear localization signal in tetracycline inducible systems have been through its addition to the tetracycline transactivator gene (tTA) (International Publication No. WO 98/10084) for use in animal and virus applications. As described elsewhere herein, the tTA system relies upon the activation potential of the VP16 tranactivating domain fused to a tet repressor gene. A similar system (tTA without the nuclear localization signal) has been shown to operate in plants (Wienmann, et al., The Plant Journal, 5(4):559-569, (1994)). Though, as referenced above, the present invention represents the first application of a nuclear localization signal to the tet repressor gene for use in plants.

The application of OCS activating elements has also been described previously for enhancing plant gene promoter activity (Ni, M., et al., The Plant Journal., 7: 661-676, (1995)). However, the application of an OCS element, and specifically a (OCS)₃ element, to a tetracycline inducible system (animal or plant) has not been described prior to the present invention.

The application of a single MAR element to a tetracycline system, specifically an operator cassette, has been described previously (Wells, K D, et al., Transgenic Res. 8(5), 371-381, (1999))—though the system is based upon the rTA system, a variant of the tTA system described supra. Although this combination of a MAR element and a tet operator cassette have been shown to operate in animals, the present invention represents the first description of one or more MAR elements, in combination with a tet repressor, tet operator, and/or tet repressor/operator cassette, for operability in plants.

The application of actin promoters in conjunction with tetracycline systems has also been described previously (International Publication No. WO 98/38322)—though the actin promoter is the human beta-actin promoter, and not the At actin-intron promoter, operable in plants, that is utilized for the present invention.

The modified tetracycline inducible cassettes are sometimes referred to herein as novel tetracycline inducible promoter cassettes, novel tetracycline inducible promoter cassettes, novel tetracycline repressor/operator cassettes, modified tetracycline inducible vectors, and modified tet-inducible system. Components of the modified tetracycline inducible cassettes are sometimes referred to herein as novel tetracycline repressor cassettes, and novel tetracycline operator cassettes. The present invention encompasses the application of each cassette, individually, or in combination, to other tetracycline inducible systems in the art. The present invention also encompasses the application of each cassette, individually, or in combination, to any other genetic construct known in the art or described herein.

### BRIEF SUMMARY OF THE INVENTION

The present invention relates to modified tetracycline-inducible promoter cassettes for controlling gene expression in organisms, particularly plants. Specifically, the present invention encompasses novel tetracycline repressor cassettes, novel tetracycline operator cassettes, and/or novel tetracycline repressor/operator cassettes.

The present invention encompasses the insertion of any of the novel tetracycline repressor, operator, and/or repressor/operator cassettes of the invention into a heterologous vector. Such a vector may be a plasmid and/or a virus, and preferably comprises a selectable marker gene, and may preferably comprise a reporter gene, and/or a gene of interest.

The invention encompasses host cells transformed with the heterologous vectors comprising any of the novel tetracycline repressor, operator, and/or repressor/operator cassettes of the present invention.

The present invention encompasses a method of identifying tetracycline analogs, and/or functional equivalents, using a modified tetracycline repressor, operator, and/or repressor/operator cassette of the present invention.

In a preferred embodiment, the modified tetracycline repressor, operator, and/or repressor/operator cassette of the present invention may be used to modulate gene expression of any gene in a plant, and/or other organism.

Moreover, the polynucleotide sequence of interest may be a polynucleotide sequence encoding a gene, an antisense polynucleotide, a ribozyme, a fusion protein, a polynucleotide encoding an antibody, etc. In specific embodiments, the polynucleotide sequence may a polynucleotide encoding a plant hormone, plant defense protein, a nutrient transport protein, a biotic association protein, any gene in an antisense orientation, a desirable input trait, a desirable output trait, a stress resistance gene, an herbicide resistance gene, in addition to other genes described elsewhere herein.

The invention encompasses a method of producing herbicide resistant plants using the modified tetracycline repressor, operator, and/or repressor/operator cassette of the present invention to control the expression of a herbicide resistance gene, for example.

Also provided is a method of expressing a gene of interest in specific plant tissues using the modified tetracycline repressor, operator, and/or repressor/operator cassette of the present invention to control the expression of a gene, for example. Deposits with the ATCC comprising the modified tetracycline repressor, operator, and/or repressor/operator cassette of the present invention are also provided.

### BRIEF DESCRIPTION OF THE FIGURES

FIG. 1—NLS-Modified tet Receptor Provides For Stronger epression of tet-Inducible System than Wild Type tet Receptor. Firefly and Renilla luciferase activities were assayed after co-electroporation of an aliquot containing 2.5×10⁶ NT1 tobacco protoplasts with 20 μg of pACAG024 (TripleX/LUC) and 15 μg of either pACAG015 ((OCS)₃MAS/nTR) or pACAG016 ((OCS)₃MAS/WtTR) followed by cultivation of half of the electroporated protoplasts in NT1 liquid medium supplemented with 3 mg/l of tetracycline, with the remaining half of the protoplasts in the same medium without inducer. Electroporation with pACRS018 (35S/LUC, see FIGS. 30A-K) was performed for control purposes. Luciferase assays were performed 24 hours later. The figure shows results of Firefly luciferase assay standardized by expression of Renilla (Firefly reading was divided by Renilla reading and multiplied by 10000). Each bar represents an average of three samples. Numbers above the bars represent magnitude of induction.

FIG. 2—Induction of GUS Gene in Leaves Expressing NLS-tet Receptor Under Control of Different Promoters. Tobacco plants. already expressing pAC499 (TripleX/GUS) were transformed with five pCAMBIA-based Agro cassettes carrying NPTII marker gene and nTR under control of 35S, 2×35S, A. thaliana HPPD, A. thaliana AHAS, A. thaliana Actin and (OCS)₃MAS promoters (pACAG076, 077, 078, 079, 109 and 084), as well as pAC489 (35S/WtTR) to yield a number of transgenic lines. This collection of tobacco plants was tested in a preliminary induction experiment performed on leaf disks: 5-10 mm leaf disks were excised from plants and put into six- or twelve-well plates with liquid MS medium and with or without Doxy 5 mg/l at 5 mg/l. GUS assays were performed with the tissues 5 days later. Results of GUS assays are presented in the figure. Each bar represents one sample; numbers above the bars represent magnitude of induction.

FIG. 3—Induction of GUS Gene In Tobacco Leaves Expressing nTR Under Control of Different Promoters. Lines that showed induction in preliminary experiments (FIG. 2) and tobacco lines transformed with AtActin/nTR were taken for advanced analysis. 5-10 mm leaf disks and pieces of meristems were excised from plants and put into six- or twelve-well plates with liquid MS medium and with or without Doxy. 5 mg/l at 5 mg/l. GUS assays were performed with the tissues 5 days later. Results of the GUS assays are presented in the figures. The figure also presents data on repression and induction in plants carrying a cassette where nTR is flanked with one or more MAR elements. Each bar represents an average of three samples; numbers above the bars represent magnitude of induction.

FIG. 4—Induction Of GUS Gene In Tobacco Meristems Expressing nTR Under Control Of Different Promoters. Lines that showed induction in preliminary experiments (FIG. 2) and tobacco lines transformed with AtActin/nTR were taken for advanced analysis. 5-10 mm leaf disks and pieces of meristems were excised from plants and put into six- or twelve-well plates with liquid MS medium and with or without Doxy 5 mg/l at 5 mg/l. GUS assays were performed with the tissues 5 days later. Results of the GUS assays are presented in the figures. The figure also presents data on repression and induction in plants carrying a cassette where nTR is flanked with one or more MAR elements. Each bar represents an average of three samples; numbers above the bars represent magnitude of induction.

FIG. 5—Reactivation of Minimal Promoters Mediated by Addition of Upstream OCS Elements. Transient Assays. This experiment was run in order to test the possibility of restoring activities of minimal promoters by addition of upstream activating elements. Cassettes with two minimal promoters, 35Sₘᵢₙ and MASₘᵢₙ located upstream of luciferase gene (pACAG088 and 089), with 35S and MAS minimal promoters with OCS elements placed at 5′ ((OCS)₃35Sₘᵢₙ and (OCS)₃MASₘᵢₙ) upstream of luciferase gene (pACAG095 and 096) and cassettes with original promoters driving luciferase gene were used in transient assays as follows. Protoplasts were isolated from NT1 tobacco cells and electroporated with each of these cassettes at concentrations of 20 μg per aliquot containing 2.5×10⁶ NT1 tobacco protoplasts. Protoplasts were transferred to NT1 liquid medium for overnight cultivation. Luciferase assays were performed 24 hours later. Results of the luciferase assays are shown in the figure. Each bar represents one sample; numbers above the bars represent magnitude of expression improvement after OCS elements were added upstream of minimal promoters.

FIG. 6—Expression of Luciferase Gene from Different Promoters in Transgenic Tobacco Plants. The following experiment was performed for the purpose of answering the question of how minimal and new chimeric promoters would work on a whole plant level. Expression of luciferase in root and leaf tissue samples from collection of transgenic tobacco plants carrying the cassettes composed of luciferase driven by minimal and chimeric promoters (pACAG105-108) were visualized by low-light video-image analysis as follows. Tissue samples in 48-well plate were overlaid with solution-containing 1 mM luciferin and 0.1% of Triton x100 followed by vacuum infiltration for 5 minutes and immediate measurement of light emission using a Night Owl LB 981. Simultaneously, a photo image of the plate was taken on the same camera. The luminescence image was converted into pseudo-color image where different colors represent different luminescence intensities (the scale is presented in the figure), and, subsequently, overlaid over the photo image. Results are shown in the figure.

FIG. 7—Expression of Luciferase Gene from Different Promoters in Transgenic Tobacco Plants. The following experiment was performed for the purpose of answering the question of how minimal and new chimeric promoters would work on a whole plant level. Expression of luciferase in whole plants from collection of transgenic tobacco lines carrying the cassettes composed of luciferase driven by minimal and chimeric promoters (pACAG105-108) were visualized by low-light video-image analysis as follows. A plant was incubated in solution containing 1 mM luciferin and 0.1% of Triton x100 under vacuum for 5 minutes followed by immediate measurement of light emission. on Night Owl LB 981. Simultaneously, a photo image of the plant was taken on the same camera. The luminescence image was converted into pseudo-color image where different colors represent different luminescence intensities (the scale is presented in the figure), and, subsequently, overlaid over the photo image. Images are collated in the figure.

FIG. 8—New tet-Responsive Promoters Provide Higher Expression of Luciferase Gene. This experiment was performed in order to test tet-inducible promoters modified by OCS elements. (OCS)₃TripleX and (OCS)₃TripleXₘᵢₙ fused to luciferase gene (pACAG042 & 050) were tested in the experiment under transient expression. Renilla and Firefly luciferase activities were assayed after co-electroporation of NT1 protoplasts with 20 μg of either pACAG042 or 050 and 15 μg of pACAG015 ((OCS)₃MAS/nTR) followed by cultivation of one half of the electroporated cells in NT1 liquid medium supplemented with 3 mg/l of tetracycline, the other half of the cells were grown in the same medium but without inducer. In control experiments (with reporter cassettes only) a plasmid similar in size to that of nTR was used to offset the effect of expression. increase caused by increase of DNA sample with addition of Receptor cassette in test experiments. Luciferase assays were performed 24 hours after electroporation. The figure shows results of Firefly luciferase assay standardized by expression of Renilla (Firefly reading was divided by Renilla reading and multiplied by a large number). Each bar represents an average of three samples. Numbers above the bars represent magnitude of induction

FIG. 9—Doxycycline-Mediated Induction of Expression of Luciferase from (OCS)₃triplexₘᵢₙ Promoter in Plants is Stronger than TripleX. This experiment was performed in order to test the performance of OCS elements-modified TripleX promoter on the whole plant level. Transgenic tobacco plants carrying (OCS)₃MAS/nTR, NPTII, and either of (OCS)₃TripleXₘᵢₙ/LUC or TripleX/LUC cassettes were tested in leaf disk induction assays: 5-10 mm leaf disks were excised from plants and put into six- or twelve-well plates with liquid MS medium and with or without Doxy 5 mg/l at 5 mg/l. Luciferase assays were performed with the tissues 5 days later. Results of the assays are presented in the figure. Each bar represents one sample; numbers above the bars represent magnitude of induction.

FIG. 10—Doxycycline-Mediated Induction of Expression of Luci erase from (OCS)₃triplexₘᵢₙ Promoter in Plants is Stronger than TripleX. Lines that showed induction in preliminary experiments (FIG. 9) were taken for advanced analysis. 5-10 mm leaf disks and pieces of roots were excised from plants and put into six- or twelve-well plates with liquid MS medium and with or without Doxy 5 mg/l at 5 mg/l. Luciferase assays were performed with the tissues 5 days later. Results of the luciferase assays are presented in the figure. Each bar represents an average of three samples; numbers above the bars represent magnitude of induction.

FIG. 11—Doxycycline-Mediated Induction of Expression of Luciferase from (OCS)₃triplexₘᵢₙ Promoter in Roots is Stronger TripleX. Lines that showed induction in preliminary experiments (FIG. 9) were taken for advanced analysis. 5-10 mm leaf disks and pieces of roots were excised from plants and put into six- or twelve-well plates with liquid MS medium and with or without Doxy 5 mg/l at 5 mg/l. Luciferase assays were performed with the tissues 5 days later. Results of the luciferase assays are presented in the figure. Each bar represents an average of three samples; numbers above the bars represent magnitude of induction.

FIG. 12—Induction Of GUS Gene in Leaves Expressing NLS-tet Receptor from Different Cassettes. This experiment was performed in order to test the. effect of one or more MAR elements on expression of nTR. Tobacco plants already expressing. pAC499 (TripleX/GUS) were transformed with pCAMBIA-based Agro cassettes carrying NPTII marker gene and nTR under the control of (OCS)₃MAS promoters with 1100 bp MAR elements or without them (pACAG049 and 084 respectively). The resulting collection of tobacco plants was tested in preliminary induction experiment performed on leaf disks: 5-10 mm leaf disks were excised from plants and put into six- or twelve-well plates with liquid MS medium with or without Doxy 5 mg/l. Luciferase assays were performed with the tissues 5 days later. Results of these assays are presented in the figure. Each bar represents one sample; numbers above the bars represent magnitude of induction.

FIG. 13—Doxycycline-Mediated Induction of Expression of Luciferase in Inducible Cassettes With or Without One or More MAR Elements. This experiment was performed in order to test the effect of placing inducible luciferase gene between one or more MAR elements. Tobacco plants carrying TripleX/LUC cassette placed between one or more MAR elements, (OCS)₃MAS/nTR and NPTII genes (pACAG073 and pACAG081) as well as the reference cassette without one or more MAR elements (pACAG085) were used as donors of 5-10 mm leaf disks which were cultivated in six- or twelve-well plates with liquid MS medium with or without doxycycline 5 mg/l for five days. Samples were assayed for luciferase activity. Results of the assays presented in the figure. Each bar represents one sample; numbers above the bars represent magnitude of induction.

FIG. 14—Doxycycline-Mediated Induction of Luciferase in Leaves Carrying Inducible Cassettes With or Without One or More MAR Elements. This experiment was performed in order to test the effect of placing inducible luciferase gene between one or more MAR elements. Lines that showed induction in preliminary experiments (FIG. 13) were taken for advanced analysis. 5-10 mm leaf disks and pieces of roots were excised from plants and put into six- or twelve-well plates with liquid MS medium and with or without Doxy 5 mg/l at 5 mg/l. Luciferase assays were performed with the tissues 5 days later. Results of the assays are presented in the figure. Each bar represents an average of three samples; numbers above the bars represent magnitude of induction.

FIG. 15—Doxycycline-Mediated Induction of Luciferase in Roots Carrying Inducible Cassettes With or Without One or More MAR Elements. This experiment was performed in order to test the effect of placing inducible luciferase gene between one or more MAR elements. Lines that showed induction in preliminary experiments (FIG. 13) were taken for advanced analysis. 5-10 mm leaf disks and pieces of roots were excised from plants and put into six- or twelve-well plates with liquid MS medium and with or without Doxy 5 mg/l at 5 mg/l. Luciferase assays were performed with the tissues 5 days later. Results of the assays are presented in the figure. Each bar represents an average of three samples; numbers above the bars represent magnitude of induction.

FIG. 16—Induction of GUS Gene in Double Transformants: Time Series. Time series were run with the best double transformants carrying pAC499 (TripleX/GUS) and either of pACAG084 ((OCS)₃MAS/nTR) or pACAG049 (MAR-(OCS)₃MAS/nTR). Three weeks-old rooted plants were transferred from agar to magenta boxes with liquid medium and tetracycline 2 mg/l. Tissue samples were regularly taken from roots and assayed for GUS. Results of the assays with root samples are presented in the figure. Each point on the graph represents an average of three samples.

FIG. 17—Using Several Tetracycline Analogs in Determining The Concentration Curve For Induction Of GUS Gene On The Protoplast Level. Two analogs—95702-03-7 and 1665-56-1—as well as tetracycline were compared for induction of the tet system in protoplast assays. Mesophyll protoplasts were isolated from T2 transgenic tobacco carrying both wild type tet Receptor and GUS gene controlled by TripleX promoter (pAC489/pAC499 double transformants) and were cultivated at a concentration of 5×10⁴ cells/ml in liquid KM medium supplemented with tet analogs at 2-5 mg/l in the dark at 26° C. Total and divided protoplasts were counted and GUS fluorescent assays were performed on the seventh day of cultivation. Toxicity of chemicals was evaluated by division rate—the number of divisions divided by the total number of viable cells and multiplied by 100. Results of the assays are presented in the figure. Each point on the graphs represents an average of three samples. Logarithmic scale is used for concentration axis.

FIG. 18—Analysis of Cyanamid's Proprietary Chemistry for Induction of the Switch. Expanded number of analogs was tested in seed germination test. Seeds of T2 homozygous tobacco carrying wild type tet Receptor and TripleX-driven GUS gene (pAC489/pAC499 double transformants) were germinated in presence,of several new analogs at different concentrations. Two-week-old seedlings were collected and assayed for GUS. Results of GUS assays are shown in the figure. Each point on the graph represents an average of three samples.

FIG. 19—Using Several Tetracycline Analogs in Determining the Concentration Curve for Induction of GUS Gene. The purpose of this experiment was to determine the concentration curve for selected analogs. Seeds of homozygous tobacco carrying wild type tet Receptor and TripleX-driven GUS gene (pAC489/pAC499 double transformants) were germinated in presence of several analogs at different concentrations. Seedlings were visually evaluated for toxicity, collected and assayed. Results of GUS assay are shown in the figure. Each point on the graph represents an average of three samples.

FIG. 20—tet-Inducible Regeneration of PURSUIT® -Resistant Shoots Followed by Infection of Tobacco With Agro Carrying pACAG029 (NLS-tet Repressor and TripleX/AHAS genes). This test was performed in order to provide quick assessment of the performance of tet-inducible AHAS gene in putative transformants carrying pACAG029 (TripleX/AHAS, (OCS)₃MAS/nTR and NPTII cassettes). During the first step of tobacco Agrobacterium-mediated transformation with pACAG029 (regeneration under selective pressure), three different selection schemes were used as follows. Leaf disks infected with Agro were placed on three different media: Kanamycin 100 mg/l alone, to select all transgenic lines; tetracycline 2 mg/l and PURSUIT® 1 μM, to select lines with highest inducible herbicide resistance; and PURSUIT® 1 μM as a control for escapes. Experiment was evaluated in three weeks. The figure shows that a number of herbicide-resistant shoots showed up on media with herbicide and tetracycline, whereas no shoots appeared on plates with herbicide alone.

FIG. 21—Induction of Herbicide Resistance in Tobacco Plants Transformed With pACAG029. This test was performed in order to provide deep evaluation of the performance of tet-inducible AHAS gene in individual tobacco plants transformed with pACAG029 (TripleX/AHAS, (OCS)₃MAS/nTR and NPTII cassettes). 17 lines selected resistant to Kanamycin were checked for induction of PURSUIT® resistance on rooting medium containing either tet 2 mg/l+PURSUIT® 1 μM or PURSUIT® 1 μM alone. Only four lines showed the induction: healthy plants with well-developed root systems grew on medium with tetracycline whereas shoots growing on the medium with herbicide only were severely inhibited. The figure shows an example of such line.

FIG. 22—tet-Inducible AHAS Gene is Repressed in Roots and True Leaves, But Not Cotyledons, of F1 Tobacco Seedlings Carrying (OCS)₃MAS-driven tet Receptor. This experiment was performed in order to test the performance of tet-inducible AHAS gene in T1 progeny of tobacco plants transformed with pACAG029 (TripleX/AHAS, (OCS)₃MAS/nTR and NPTII cassettes). T1 seeds were plated on media with either PURSUIT® 1 μM alone, or PURSUIT® 1 μM and Doxycycline 3 mg/l. Two weeks later, all seed lines germinated on both media and produced green cotyledons, though after closer evaluation it was noted that roots are severely inhibited on seedlings of only one line, #4, growing on PURSUIT® alone compared to no root inhibition on the same media with Doxycycline (figure shows how the seedlings looked from the bottom of the plate). Further evaluation revealed that true leaves are also inhibited on these seedlings (examples of the plantlets are shown in the figure).

FIG. 23—Induction of Herbicide Resistance in Transgenic Tobacco Works Equally Well in Both T1 And T2 Progenies. This experiment was performed in order to test the performance of tet-inducible AHAS gene in T2 progeny of transgenic tobacco plants homozygous for pACAG029 (TripleX/AHAS, (OCS)₃MAS/nTR and NPTII cassettes). T1 heterozygous and T2 homozygous seeds of the line #4 were germinated on MS plates supplemented with 5 μM of PURSUIT® either alone or with doxycycline 5 mg/l. Images of plates were taken two weeks later and are shown in the figure.

FIG. 24—Induction of Herbicide Resistance in Tobacco Plants: Leaf Disk Test. A concentration curve test was run using the tobacco line that showed the best tet-inducible herbicide resistance (pACAG029 #4). Leaf discs from T1 plant were floated on liquid medium supplemented with PURSUIT® 1, 3, 10 and 20 μM either alone or with doxycycline 10 mg/l. Three weeks later the inhibition of tissues was evaluated visually. The inhibition occurred as bleaching of tissues compared to the green color of healthy leaf disks, and smaller size of the disk compared to expanded healthy tissues. The figure shows the differences among tissues treated with different chemicals.

FIG. 25—Induction of Herbicide Resistance in Transgenic Tobacco: Regeneration Test. This test was performed in order to provide deep evaluation of the performance of tet-inducible AHAS gene in individual tobacco plants transformed with pACAG029, 119, 119r, 120, and 120r (all carrying AtActin/nTR and NPTII cassettes and either TripleX or (OCS)₃TripleXₘᵢₙ promoter driving AHAS gene). Leaf disks from tobacco plants under study and pACAG029 #4, line that showed good induction of PURSUIT® resistance before, were placed on agar supplemented with 1 mg/l of BAP and 5 μM of PURSUIT® either alone or with doxycycline 5 mg/l. Three weeks later the inhibition of tissues was evaluated visually. The inhibition occurred as lack of regeneration events compared to multiple shoots growing up on healthy explants, bleaching of tissues compared to the green color of healthy leaf disks, and smaller size of the disk compared to expanded healthy tissues. The figure shows the-differences between tissues treated or untreated with doxycycline for several lines.

FIG. 26—Induction of Herbicide Resistance in Transgenic Arabidopsis Leaves Carrying pACAG029. This test was performed in order to provide quick assessment of the performance of tet-inducible AHAS gene in individual Arabidopsis plants transformed with pACAG029 (TripleX/AHAS, (OCS)₃MAS/nTR and NPTII cassettes). Fifteen Arabidopsis plants selected resistant to Kanamycin after transformation with pACAG029 were checked for induction of PURSUIT®resistance by cultivation of a single leaf from these plants in MS liquid medium containing either tetracycline 2mg/l and PURSUIT® 1 μM or PURSUIT® 1 μM alone. The test was evaluated two weeks later. Only two lines showed the induction: green, healthy leaf grew on medium with tetracycline and PURSUIT®whereas severely inhibited leaf grew in the medium with herbicide alone. These two lines are presented in the figure.

FIG. 27—Induction of Herbicide Resistance in Arabidopsis Plants: Seed Germination Test. This test was performed in order to provide deep evaluation of the performance of tet-inducible AHAS gene in individual Arabidopsis plants transformed with pACAG029 (TripleX/AHAS, (OCS)₃MAS/nTR and NPTII cassettes). Seeds from 17 plants selected resistant to Kanamycin were planted on media with either PURSUIT® 1 μM alone or PURSUIT® 1 μM and tetracycline at 2 mg/l. The test was evaluated two weeks later. Five lines showed induction of herbicide resistance on the medium with tetracycline. The best line, AG029A # 4, which had the least number of escapes on the medium with herbicide alone, is shown in the figure.

FIG. 28—Induction Of Herbicide Resistance in Transgenic Arabidopsis Works Better in Homozygous Line. This experiment was run in order to compare inducibility in heterozygous versus homozygous lines transformed with pACAG029 (TripleX/AHAS, (OCS)₃MAS/nTR and NPTII cassettes). Five Arabidopsis lines transformed with pACAG029, T1 heterozygous and T2 homozygous seeds, were germinated on MS plates supplemented with 5 μM of PURSUIT® either alone or with doxycycline 5 mg/l. The test was evaluated two weeks later. Results with one of these lines, #1, are shown in the figure.

FIG. 29A-F—Shows the polynucleotide sequences of the wild-type tet repressor coding region (Wt TR—SEQ ID NO:1); the polynucleotide sequence of the SV40 Large T-antigen nuclear localization sequence (SV40 NLS—SEQ ID NO:2), see Boulikas, T. (1993); the polynucleotide sequence of the tet repressor coding region operably linked to the SV40 Large T-antigen nuclear localization signal (nTR—SEQ ID NO:3); the polynucleotide sequence of the tet Triple X operator (TripleX—SEQ ID NO:4); the polynucleotide sequence of an upstream OCS activator sequence (OCS—SEQ ID NO:5); the polynucleotide sequence of three tandem OCS activator sequences ((OCS)₃—SEQ ID NO:6); the polynucleotide sequence of the TripleX minimal promoter (TripleXₘᵢₙ—SEQ ID NO:7); the polynucleotide sequence of the TripleX minimal promoter downstream from the three upstream OCS activator sequences ((OCS)₃TripleXₘᵢₙ—SEQ ID NO:8); the polynucleotide sequence of the matrix attachment region (MAR—SEQ ID NO:9); the polynucleotide sequence of the CtToo 35Sₘᵢₙ promoter (SEQ ID NO:10); the polynucleotide sequence of the CtTtt 35Sₘᵢₙ promoter (SEQ ID NO:11); the polynucleotide sequence of the CtTttt 35Sₘᵢₙ promoter (SEQ ID NO:12); the polynucleotide sequence of the CtTot 35Sₘᵢₙ promoter (SEQ ID NO:13); the polynucleotide sequence of the CoToo 35Sₘᵢₙ romoter (SEQ ID NO:14); the polynucleotide sequence of the CoTtt 35Sₘᵢₙ promoter (SEQ ID NO:15).; the polynucleotide sequence of the CtTto 35Sₘᵢₙ promoter (SEQ ID NO:16); the polynucleotide sequence of the CoTot 35Sₘᵢₙ promoter (SEQ ID NO:17); the polynucleotide sequence of the CoTott 35Sₘᵢₙ promoter (SEQ ID NO:18); the polynucleotide sequence of the CoTto 35Sₘᵢₙ promoter (SEQ ID NO:19); the polynucleotide sequence of the CTo MASₘᵢₙ promoter (SEQ ID NO:20); the polynucleotide sequence of the tCTo MASₘᵢₙ promoter (SEQ ID NO:21); the polynucleotide sequence of the CTTttTTt MASₘᵢₙ promoter (SEQ ID NO:22); the polynucleotide sequence of the tCTtT MASₘᵢₙ promoter (SEQ ID NO:23); the polynucleotide sequence of the CTo MASₘᵢₙ promoter (SEQ ID NO:19); and the polynuceotide sequence of the various promoter element fragments used in constructing the 35S- and MAS-based promoter cassettes of the present invention (SEQ ID NOS: 24 thru 36).

FIGS 30A—Shows the structural schemes for the various promoter cassettes and cassettes of the present invention. Abbreviations for the 35S- and MAS-based promoter cassettes are as follows: Capital “C” equals “CAAT” box, Capital “T”. equals “TATA” box, Lower case “t” equals “tet operator”, and Lower case “o” stands for “transcriptionally nonfunctional DNA”.

FIG. 31—Shows a schematic representation of the annealed oligonucleotide fragments used to create the 35S and MAS-based promoter cassettes of the present invention (see Example 1 for the method of assembly for creating each cassettes and FIGS. 30A-K for the polynucleotide sequence of each of the individual fragments).

FIG. 32—Doxycycline-Mediated Induction Of Expression Of Luciferase From 35S-Based Inducible Promoters In Plant Protoplasts. Cassettes containing the (OCS)₃TripleX or (OCS)₃TripleXₘᵢₙ promoters were fused to the coding region of the luciferase gene (pACAG042 & pACAG050) and their resulting expression analyzed through transient expression. Firefly luciferase activity was assayed after co-electroporation of NT1 protoplasts with 20 μg of either pACAG042 or pACAG050 and 15 μg of pACAG015 ((OCS)₃MAS/nTR). One half of the transformed protoplasts were then cultivated in NT1 liquid medium supplemented with 3 mg/l of tetracycline, while the remaining half were cultivated in the same medium without inducer. Luciferase assays were performed 24 hours after electroporation. The figure shows results of Firefly luciferase assay standardized by amount of protein in the sample. Each bar represents an average of three samples. Numbers above the bars represent magnitude of induction. Each promoter is shown as a schema representing the relative location of the most important transcription elements (OCS enhancer, TATA and CAAT boxes and tet operators). Each of the tet operator sequence locations are labeled as “A”, “B”, “C”, or “D”. The schema for each cassette is drawn with 5′ to 3′ represented in the left to right direction.

FIG. 33—Doxycycline-Mediated Induction Of Expression Of Luciferase From 35S-Based Inducible Promoters In Plants. Transgenic tobacco plants carrying ((OCS)₃MAS/nTR, NPTII, in addition to a cassette containing one of the novel promoter cassettes of the present invention driving the expression of the LUC coding region) or TripleX/LUC cassettes were tested in leaf disk induction assays:: 5-10 mm leaf disks were excised from plants and put into six- or twelve-well plates with liquid MS medium and with or without Doxy 5 mg/l at 5 mg/l. Luciferase assays were performed with the tissues 5 days later. Results of the assays are shown. Each bar represents one sample; numbers above the bars represent magnitude of induction. The schema for each cassette is drawn with 5′ to 3′ represented in the left to right direction.

FIG. 34—Doxycycline-Mediated Induction Of Expression Of Luciferase From MAS-Based Inducible Promoters In Plant Protoplasts. (OCS)₃TripleX and (OCS)₃TripleXₘᵢₙ fused to coding region of the luciferase gene (pACAG042 & 050) were analyzed for expression in transient expression assays. Firefly luciferase activity was assayed after co-electroporation of NT1 protoplasts with 20 μg of either pACAG042 or pACAG050 and 15 μg of pACAG015 ((OCS)₃MAS/nTR). One half of the electroporated protoplasts were then cultivated in NT1 liquid medium supplemented with 3 mg/l of tetracycline, with the remaining half being cultivated in the same medium without inducer. Luciferase assays were performed 24 hours after electroporation. The figure shows results of the Firefly luciferase assay standardized by amount of protein in the sample. Each bar represents an average of three samples. Numbers above the bars represent magnitude of induction. The schema for each cassette is drawn with 5′ to 3′ represented in the left to right direction.

FIG. 35—tet-Inducible Herbicide Resistance: Cassette Orientation Effects. Individual Arabidopsis lines were transformed with either pACAG119, pACAG119r, pACAG120, or pACAG120r. Each cassette comprised the AtActin/nTR and NPTII cassettes and either TripleX or (OCS)₃TripleXₘᵢₙ promoter driving AHAS gene—differing only in the relative orientation of each cassette within the vector. Seeds from plants selected to be Kanamycin resistant were planted on media with either PURSUIT® 1 μM alone or PURSUIT® 1 μM and doxycycline at 5 mg/l. The results of the experiment were evaluated two weeks later. All lines showed different level of induction of herbicide resistance on the medium with doxycycline. Since results were highly consistent for lines representing the same cassette, it was possible to attribute typical response patterns of plants to the vector used for transformation. The most typical responses for each cassette are shown in the figure. In the case of the pACAG119 cassette, two typical responses were detected, each of which are represented by the top and bottom photograph sets in the figure. The control cassette, AG029A # 4, was also tested in this experiment, though results are not shown in the figure.

FIG. 36—tet-Inducible Herbicide Resistance: Spray Test of F1 Seedlings and Seeds of Tobacco Line pACAG029#4. Seedlings at different stages of development (1 and 2 weeks old), produced by germination in 2.5″×2.5″ pots with Metro mix, were used for assessing tet inducible herbicide resistance in a post-emergence test. For pre-emergency application, seeds were sown on the Metro mix right before PURSUIT®, application. 15-20 seeds were placed in each pot. Pots were sprayed with doxycycline premixed with PURSUIT®, each at different rates. The test was evaluated two weeks after the spray. Each pot in the figure represents a unique experiment in terms of the combination of chemical rates and the stage of seedling development. As shown in the figure, the rate of herbicide resistance directly corresponded with increased doxycycline application rates.

### DEFINITIONS

The description that follows uses a number of terms that refer to recombinant DNA technology. In order to provide a clear and consistent understanding of the specification and claims, including the scope to be given such terms, the following definitions are provided.

Expression vector: This and comparable terms refer to a vector which is capable of inducing the expression of DNA that has been cloned into it after transformation into a host cell. The cloned DNA is usually placed under the control of (i.e., operably linked to) certain regulatory sequences such a promoters or enhancers. Promoters sequences maybe constitutive, inducible or repressible.

Host: Any prokaryotic or eukaryotic cell that is the recipient of a vector is the host for that vector. The term encompasses prokaryotic or eukaryotic cells that have been engineered to incorporate a gene in their genome. Cells that can serve as hosts are well known in the art as are techniques for cellular transformation (see e.g., Sambrook, et al., Molecular Cloning: A Laboratory Manual, 2nd ed., Cold Spring Harbor (1989)).

Promotor: A DNA sequence that initiates the transcription of a gene. Promoters are typically found 5′ to the gene and located proximal to the start codon. If a promotor is of the inducible type (i.e., the tetracycline inducible promoters of the present invention), then the rate of transcription increases in response to an inducing agent.

Minimal Promotor: A DNA sequence that initiates the transcription of a gene that may have less than the original elements found in the parent promoter, though still maintains the ability to initiate transcription (e.g., enhancer, binding domains, regulatory domains, etc.).

Expression: Expression is the process by which a polypeptide is produced from DNA. The process involves the transcription of the gene into mRNA and the translation of this mRNA into a polypeptide. Depending on the context in which it is used, the term “expression” may refer to the production of RNA, protein or both.

Repressor: As used herein, the term “repressor” refers to a molecule capable of inhibiting the expression of a particular gene from a promoter. In effect, the molecule “represses” the expression of the gene from its promoter. For example, the tet repressor is a protein that represses gene transcription of the tet operon upon binding to its cognate tet operator sequences within the operon promoter.

Derepression: As used herein, the term “derepression” may be construed to mean the reversal of “repression”. If the expression of a gene is repressed, then upon “derepression”, transcriptional would be activated and the gene expressed, for example.

Recombinant: As used herein, the term “recombinant” refers to nucleic acid that is formed by experimentally recombining nucleic acid sequences and sequence elements. A recombinant host would be any host receiving a recombinant nucleic acid and the term “recombinant protein” refers to protein produced by such a host.

Operably linked: The term “operably linked” refers to genetic elements that are joined in such a manner that enables them to carry out their normal functions. For example, a gene is operably linked to a promotor when its transcription is under the control of the promotor and such transcription produces the protein normally encoded by the gene.

Gene: As used herein, “gene” refers to the nucleic acid sequence that undergoes transcription as the result of promoter activity. A gene may code for a particular protein or, alternatively, code for an RNA sequence that is of interest in itself, e.g. because it acts as an antisense inhibitor.

Transcriptionally Silent DNA: As used herein, DNA referred to as being “transcriptionally silent” is a reference to that particular DNA lacking the required elements to initiate transcription, such as promoter elements, enhancers, TATA boxes, etc. The related terms, “DNA without function” and “inactive DNA” should be construed as having the same meaning and may be referred to herein.

PURSUIT®: As used herein, “PURSUIT®” refers to a commercial formulation of the herbicide imazethapyr containing 240 mg/mL active ingredient, manufactured by American Cyanamid Co.).

Several chemical compounds are referenced herein. Each number references the chemical compounds American Chemical Society (ACS) registry number. In this instance, each of the compounds have previously been described in the art as being a tetracycline analog. Specifically, ACS Reg. No. 1665-56-1 is disclosed in U.S. Pat. No. 2,990,426; ACS Reg. No. 4199-33-1 is disclosed in U.S. Pat. Nos. 3,030,377, 3,093,549 and 3,146,264; ACS Reg. No. 95702-03-7 is disclosed in Conover, L. H., et al., J. Am. Chem. Soc., 84:3222-4 (1962); ACS Reg. No. 4497-08-9 is disclosed in U.S. Pat. No. 2,744,931; ACS Reg. No. 101057-85-6 is disclosed in Hlavka, J. J., et al., J. Am. Chem. Soc., 84:1426-30 (1962); and ACS Reg. No. 64-73-3 is disclosed in U.S. Pat. No. 3,255,079.

### DETAILED DESCRIPTION OF THE INVENTION

The novel tetracycline repressor cassettes of the present invention comprises a promoter operably linked to the tetracycline repressor coding sequence (SEQ ID NO:1), a transcriptional terminator sequence, and one or more enhancer sequences and/or a nuclear localization signal. The promoters for the novel tetracycline repressor cassettes of the present invention are preferably selected from the group consisting of: the mannopine synthase promoter (MAS), the minimal MAS-promoter, the Arabidopsis thaliana acetohydroxyacid synthase promoter (AtAHAS), the Arabidopsis thaliana hydroxyphenylpyruvate dioxygenase promoter (AtHPPD), the Arabidopsis thaliana Actin-Intron promoter, the Cauliflower Mosaic Virus 35s promoter, the two tandem CMV promoters—2×35s (R. Kay et al.,1987), and the 35s minimal promoter. Other promoters are encompassed by the invention and are described elsewhere herein.

The enhancer sequences of the novel tetracycline repressor cassettes of the present invention may be located upstream (5′) of the promoter, downstream (3′) of the tetracycline repressor gene, or both, and comprise the octopine synthase upstream activating sequence (SEQ ID NO:5), and the matrix attachment regions (MAR) (SEQ ID NO:9). Preferably, the novel tetracycline repressor cassettes of the invention comprise one, two, three, or more OCS elements in tandem, upstream of the promoter. Most preferred are novel tetracycline repressor cassettes of the invention comprising three OCS elements in tandem (SEQ ID NO:6) upstream of the promoter. Also preferred, are the novel tetracycline repressor cassettes of the present invention comprising a plurality of OCS elements and one or more MAR elements, preferably, comprising a MAR element (SEQ ID NO:9) and three OCS elements in tandem (SEQ ID NO:6) located upstream of the promoter.

As referenced above, the novel tetracycline repressor cassettes of the present invention preferably comprise a nuclear localization signal, functional in plant cells, operably linked to the tetracycline repressor coding region (SEQ ID NO:3). The localization signal is preferably from the SV40 Large T-antigen (Boulikas, infra) (SEQ ID NO:2).

The transcriptional terminator sequences of the novel tetracycline repressor cassettes of the present invention preferably comprise a terminator operable in plants and operably linked to the 3′ end of the tetracycline repressor coding region. The terminator sequences may be selected from the group consisting of the NOS terminator, and the OCS terminator. Other terminator sequences are known in the art and are encompassed by the invention. The skilled artisan would appreciate that any terminator known in the art could be used to substitute the terminator sequence of the present invention using known molecular biology techniques.

In a preferred embodiment, the novel tetracycline repressor cassettes of the present invention comprise one or more MAR elements (SEQ ID NO:9) and three tandem OCS elements (SEQ ID NO:6) located upstream of the MAS promoter, the SV40 Large T-antigen operably linked to the tetracycline repressor coding region, and the NOS terminator.

The present invention also encompasses novel tetracycline operator cassettes. As referenced previously, prior tetracycline-inducible systems operable in plants relied upon the 35s promoter to modulate the expression of a gene of interest. This promoter is not only a weak promoter, but results in gene expression in the majority of plant tissue due to its constitutive activity. The novel tetracycline operator cassettes of the present invention overcome these problems by integrating enhancer sequences upstream of the tet operator promoter, and utilizing promoters other than the wild-type 35s promoter, for example. As discussed more specifically herein, such enhancer sequences not only significantly increase gene expression from the tet operator promoter, but also provide decreased leaky expression, and surprisingly distinct tissue-specific expression.

The novel tetracycline operator cassettes preferably comprise a promoter comprising tetracycline operator sequences (SEQ ID NO:4) operably linked to the coding sequence of a gene of interest, transcriptional terminator sequences, and enhancer sequences. The promoters for the novel tetracycline operator cassettes of the present invention are preferably selected from the group consisting of: the tetracycline TripleX promoter with the −540 to −64 fragment of the 35S promoter located upstream of a consensus tetracycline operator region, the minimal tetracycline TripleX promoter with the −89 to −64 fragment (SEQ ID NO:7) of the 35S promoter located upstream of a consensus tetracycline operator region, the 35S promoter comprising at least one tet operator sequence, the MAS promoter comprising at least one tet operator sequence, and the minimal MAS promoter comprising at least one tet operator sequence. Other promoters are encompassed by the invention and are described elsewhere herein.

The enhancer sequences of the novel tetracycline operator cassettes of the present invention may be located upstream (5′) of the promoter, downstream (3′) of the gene of interest, or both, and comprise the octopine synthase upstream activating sequence (SEQ ID NO:5), and the matrix attachment regions (MAR). (SEQ ID NO:9). Preferably, the novel tetracycline operator cassettes of the invention comprise one, two, three, or more OCS elements in tandem, upstream of the promoter. Most preferred are novel tetracycline operator cassettes of the invention comprising three OCS elements in tandem (SEQ ID NO:6) upstream of the promoter. Also preferred, are the novel tetracycline operator cassettes of the present invention comprising a plurality of OCS elements and one or more MAR elements (SEQ ID NO:9), preferably, comprising one or more MAR elements and three OCS elements in tandem (SEQ ID NO:6) located upstream of the promoter.

The transcriptional terminator sequences of the novel tetracycline operator cassettes of the present invention preferably comprise a terminator operably linked to the 3′ end of the gene of interest coding region, and preferably operable in plants. The terminator sequences may be selected from the group consisting of the NOS terminator, and the OCS terminator. Other terminator sequences are known in the art and are encompassed by the invention. The skilled artisan would appreciate that any terminator known in the art could be used to substitute the terminator sequence of the present invention using known molecular biology techniques.

A preferred embodiment of the present invention is a novel tetracycline operator cassette of the present invention comprising one or more MAR elements (SEQ ID NO:9) and three OCS tandem elements (SEQ ID NO:6) located upstream of the minimal TripleX promoter (SEQ ID NO:7), a polynucleotide sequence of interest, and the OCS terminator.

Another preferred embodiment of the present invention is a novel tetracycline operator cassette of the present invention comprising three OCS tandem elements (SEQ ID NO:6) located upstream of, and operably linked to, a 35S promoter comprising at least one tet operator sequence (SEQ ID NO:4), preferably at least two, preferably at least three, or preferably at least four or more, tet operator sequences (SEQ ID NO:4), a polynucleotide sequence of interest, and the OCS terminator.

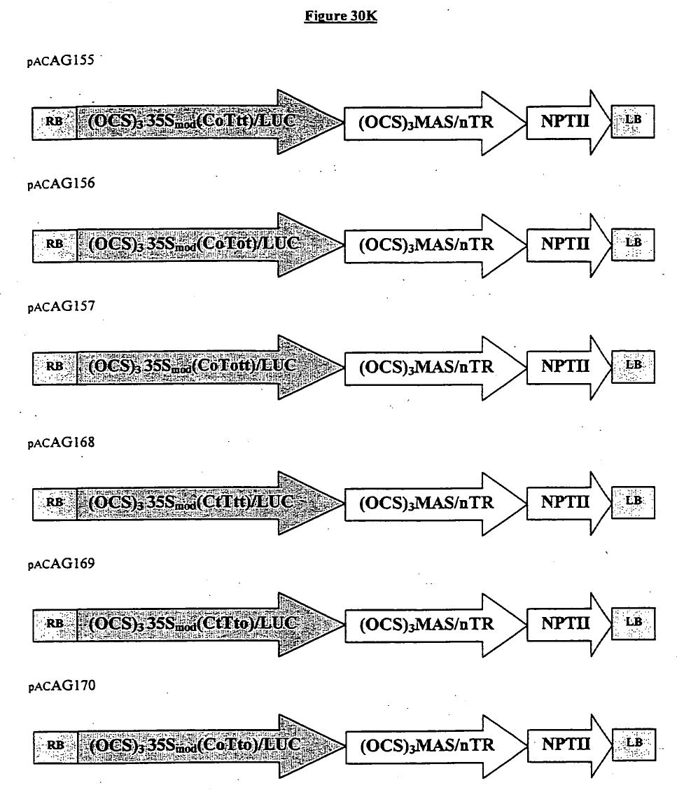

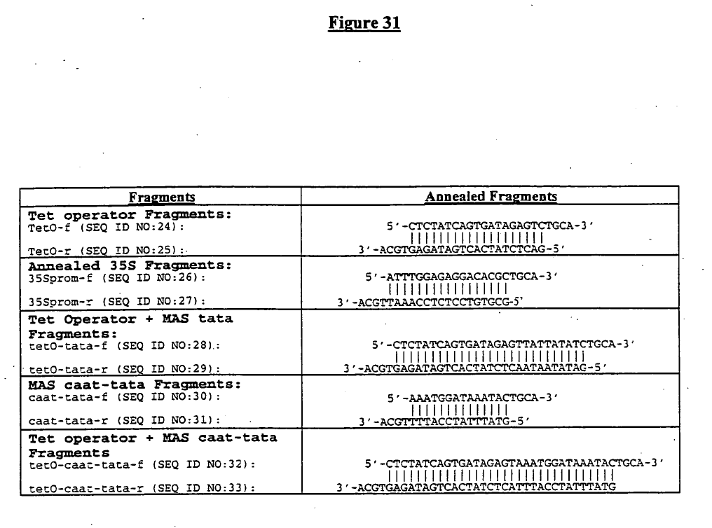

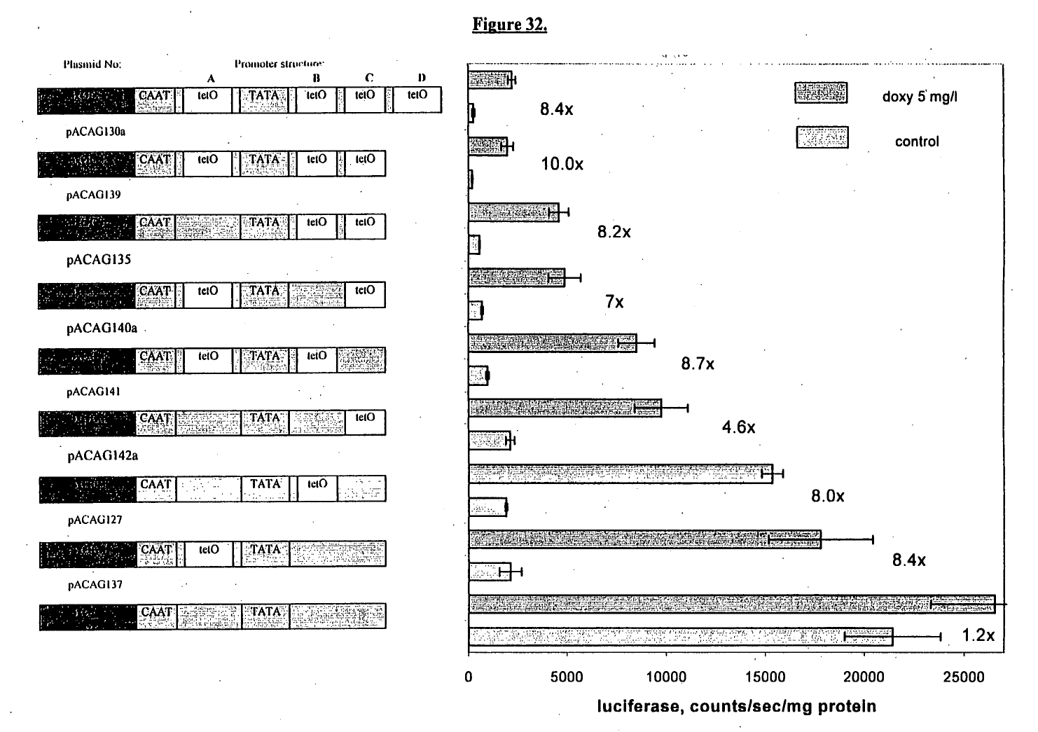

Yet another preferred embodiment of the present invention is a novel tetracycline operator cassette of the present invention comprising three OCS tandem elements (SEQ ID NO:6) located upstream of, and operably linked to, a 35S promoter comprising at least one tet operator sequence (SEQ ID NO:4), preferably at least two, preferably at least three, or preferably at least four or more, tet operator sequences (SEQ ID NO:4), wherein the at least one, two, three, four or more tet operator sequences (SEQ ID NO:4) are positioned such that the tet operator location indicated as site “A” in FIG. 32 is occupied by one of the tet operator sequences, a polynucleotide sequence of interest, and the OCS terminator.

Another preferred embodiment of the present invention is a novel tetracycline operator cassette of the present invention comprising three OCS tandem elements (SEQ ID NO:6) located upstream of, and operably linked to, a MAS promoter comprising at least one tet operator sequence (SEQ ID NO:4), preferably at least two, preferably at least three, or preferably at least four or more, tet operator sequences (SEQ ID NO:4), a polynucleotide sequence of interest, and the OCS terminator.

Yet another preferred embodiment of the present invention is a novel tetracycline operator cassette of the present invention comprising three OCS tandem elements (SEQ ID NO:6) located upstream of, and operably linked to, a MAS minimal promoter comprising at least one tet operator sequence (SEQ ID NO:4), preferably at least two, preferably at least three, or preferably at least four or more, tet operator sequences (SEQ ID NO:4), a polynucleotide sequence of interest, and the OCS terminator.

The novel tetracycline operator cassette of the present invention encompasses the modulation of more than one polynucleotide sequence of interest (preferably a polynucleotide sequence encoding a gene), under the control of the operator promoter.

The present invention also encompasses novel tetracycline repressor/operator cassettes. The tetracycline repressor/operator cassettes of the present invention comprise at least one novel tetracycline repressor cassette of the present invention located on the same plasmid and/or vector as at least one novel tetracycline operator cassette of the present invention. The invention encompasses the novel tetracycline repressor/operator cassettes whereby at least one novel tetracycline repressor cassette of the present invention is located upstream or downstream of at least one novel tetracycline operator cassette of the present invention—where both the repressor and operator cassettes are oriented in the same 5′ to 3′ direction (i.e., transcription from the promoter of both of the cassettes proceeds in the same direction). Alternatively, the invention encompasses novel tetracycline repressor/operator cassettes whereby at least one novel tetracycline repressor cassette of the present invention is located upstream or downstream of at least one novel tetracycline operator cassette of the present invention—whereby the repressor and operator cassettes are oriented in opposing directions (i.e., transcription from the promoter of both of the cassettes proceeds in opposite directions). Preferably the novel tetracycline repressor cassette and the novel tetracycline operator cassette are separated by a suitable polynucleotide spacer that is transcriptionally silent. Alternatively, the polynucleotide spacer may be transcriptionally active (e.g., may encode one or more genes, may be an active promoter, etc.).

Specifically, the tetracycline repressor/operator cassettes of the present invention comprise the following elements: a first promoter, operably linked to the tetracycline repressor coding sequence (SEQ ID NO:1), a first transcriptional terminator sequence, first and/or second enhancer sequences, the tetracycline repressor coding sequence (SEQ ID NO:2), or optionally a nuclear localization signal operably linked to the tetracycline repressor coding sequence (SEQ ID NO:3), a second promoter containing tetracycline operator sequences (SEQ ID NO:4), a polynucleotide sequence of interest, third and/or forth enhancer sequences, and a second terminator sequence. Preferably the first transcriptional terminator sequence, the first and/or second enhancer sequences, and the optional nuclear localization signal are associated with the first promoter, and the third and/or forth enhancer sequences are associated with the second promoter with the tetracycline operator sequences. Additionally, the polynucleotide sequence of interest is under the control of the second promoter with the tetracycline operator sequences. The invention encompasses the control of more than one polynucleotide of interest., preferably a gene, under the control of the second promoter.

The first promoter for the tetracycline repressor/operator cassettes of the present invention are preferably selected from the group consisting of: the mannopine synthase promoter (MAS), the minimal MAS promoter, the Arabidopsis thaliana acetohydroxyacid synthase promoter (AtAHAS), the Arabidopsis thaliana hydroxyphenylpyruvate dioxygenase promoter (AtHPPD), the Arabidopsis thaliana Actin-Intron promoter, the Cauliflower Mosaic Virus 35s promoter, the two tandem CMV promoters—2×35s (R. Kay et al.,1987), and the 35s minimal promoter. Other promoters are encompassed by the invention and are described elsewhere herein.

The first and second enhancer sequences of the novel tetracycline repressor/operator cassettes of the present invention may be located upstream (5′) of the promoter, downstream (3′) of the tetracycline repressor gene, or both, and comprise the octopine synthase upstream activating sequence (SEQ ID NO:5), and the matrix attachment regions (MAR) (SEQ ID NO:9). Preferably, the novel tetracycline repressor/operator cassettes of the invention comprise one, two, three, or more OCS elements in tandem, upstream of the promoter. Most preferred are novel tetracycline repressor/operator cassettes of the invention comprising three OCS elements in tandem (SEQ ID NO:6) upstream of the promoter. Also preferred, are novel tetracycline repressor/operator cassettes of the present invention comprising a plurality of OCS elements and one or more MAR elements, preferably, comprising a MAR element (SEQ ID NO:9) and three OCS elements in tandem (SEQ ID NO:6) located upstream of the promoter.

As referenced above, the novel tetracycline repressor/operator cassettes of the present invention preferably comprise a nuclear localization signal, functional in plant cells, operably linked to the tetracycline repressor coding region (SEQ ID NO:3). The localization signal is preferably from the SV40 Large T-antigen (Boulikas, infra) (SEQ ID NO:2).

The first transcriptional terminator sequences of the novel tetracycline repressor/operator cassettes of the present invention preferably comprise a terminator operable in plants and operably linked to the 3′ end of the tetracycline repressor coding region. The terminator sequences may be selected from the group consisting of the NOS terminator, and the OCS terminator. Other terminator sequences are known in t he art and are encompassed by the invention. The skilled artisan would appreciate that any terminator known in the art could be used to substitute the terminator sequence of the present invention using known molecular biology techniques.

The second promoter of the novel tetracycline repressor/operator cassettes preferably are selected from the group consisting of: the tetracycline TripleX promoter with the −540 to −64 fragment of the 35S promoter located upstream of a consensus tetracycline operator region, the minimal tetracycline TripleX promoter with the −89 to −64 fragment of the 35S promoter located upstream of a consensus tetracycline operator region (SEQ ID NO:7), the 35S promoter comprising at least one tet operator sequence, the MAS promoter comprising at least one tet operator sequence, and the minimal MAS promoter comprising at least one tet operator sequence. Other promoters are encompassed by the invention and are described elsewhere herein.

The third and forth enhancer sequences of the novel tetracycline repressor/operator cassettes of the present invention may be located upstream (5′) of the second promoter, downstream (3′) of the polynucleotide sequence of interest, or both, and comprise the octopine synthase upstream activating sequence (SEQ ID NO:5), and the matrix attachment regions (MAR) (SEQ ID NO:9). Preferably, the novel tetracycline repressor/operator cassettes of the invention comprise one, two, three, or more OCS elements in tandem, upstream of the second promoter. Most preferred are novel tetracycline repressor/operator cassettes of the invention comprising three OCS elements in tandem (SEQ ID NO:6) upstream of the second promoter. Also preferred, are the novel tetracycline repressor/operator cassettes of the present invention comprising a plurality of OCS elements and one or more. MAR elements, preferably, comprising a MAR element (SEQ ID NO:9) and three OCS elements in tandem (SEQ ID NO:6) located upstream of the second promoter.

The second transcriptional terminator sequences of the novel tetracycline repressor/operator cassettes of the present invention preferably comprise a terminator operably linked to the 3′ end of the polynucleotide sequence of interest coding region, and preferably operable in plants. The terminator sequences may be selected from the group consisting of the NOS terminator, and the OCS terminator. Other terminator sequences are known in the art and are encompassed by the invention. The skilled artisan would appreciate that any terminator known in the art could be used to substitute the terminator sequence of the present invention using known molecular biology techniques.

In a preferred embodiment., the novel tetracycline repressor/operator cassette of the present invention comprises a first MAR element (SEQ ID NO:9) and a first three tandem OCS element (SEQ ID NO:6) located upstream of a MAS promoter, the SV40 Large T-antigen nuclear localization signal operably linked to the tetracycline repressor coding region (SEQ ID NO:3) and the NOS terminator, a second MAR element (SEQ ID NO:9) and a second three OCS tandem element (SEQ ID NO:6) located upstream of the minimal TripleX promoter (SEQ ID NO:7), a polynucleotide sequence of interest and the OCS terminator wherein the transcription of the MAS and minimal TripleX promoters are transcriptionally in the same direction. Alternatively, the MAS and minimal TripleX promoters are transcriptionally in opposing directions.

In a preferred embodiment, the novel tetracycline repressor/operator cassette of the present invention comprises a first MAR element (SEQ ID NO:9) and a first three tandem OCS element (SEQ ID NO:6) located upstream of a MAS promoter, the SV40 Large T-antigen nuclear localization signal operably linked to the tetracycline repressor coding region (SEQ ID NO:3) and the NOS terminator, a second MAR element (SEQ ID NO:9) and a second three OCS tandem element (SEQ ID NO:6) located upstream of a 35S promoter comprising at least one tet operator sequence (SEQ ID NO:4), preferably at least two, preferably at least three, or preferably at least four or more, tet operator sequences (SEQ ID NO:4), a polynucleotide sequence of interest and the OCS terminator wherein the transcription of the MAS and minimal TripleX promoters are transcriptionally in opposing direction. Alternatively, the MAS and the 35S promoter with at least one tet operator sequence, are transcriptionally in the same direction. Alternatively, the 35S promoter is the 35S minimal promoter with at least one, preferably at least two, three, four, or more tet operator sequences.

In a preferred embodiment, the novel tetracycline repressor/operator cassette of the present invention comprises a first MAR element (SEQ ID NO:9) and a first three tandem OCS element (SEQ ID NO:6) located upstream of a MAS promoter, the SV40 Large T-antigen nuclear localization signal operably linked to the tetracycline repressor coding region (SEQ ID NO:3) and the NOS terminator, a second MAR element (SEQ ID NO:9) and a second three OCS tandem element (SEQ ID No:6) located upstream of a second MAS promoter comprising at least one tet operator sequence (SEQ ID NO:4), preferably at least two, preferably at least three, or preferably at least four or more, tet operator sequences (SEQ ID NO:4), a polynucleotide sequence of interest and the OCS terminator wherein the transcription of the first MAS and second MAS promoters are transcriptionally in opposing direction. Alternatively, the MAS and minimal TripleX promoters are transcriptionally in the same direction. Alternatively, the MAS promoter is the MAS minimal promoter with at least one, preferably at least two, three, four, or more tet operator sequences.

Alternatively, the present invention encompasses novel tetracycline repressor, operator, and/or repressor/operator cassettes of the invention which do not contain an SV40 nuclear localization signal (SEQ ID NO:2).

The present invention encompasses the insertion of any of the novel tetracycline repressor, operator, and/or repressor/operator cassettes of the invention into a heterologous vector. Such a vector may be a plasmid and/or a virus, and preferably comprises a selectable marker gene, and may preferably comprise a reporter gene.

The invention encompasses host cells transformed with the heterologous vector comprising any of the novel tetracycline repressor, operator, and/or repressor/operator cassettes of the present invention.

The present invention encompasses a method of identifying tetracycline analogs, and/or functional equivalents, using a modified tetracycline repressor, operator, and/or repressor/operator cassette of the present invention.

In a preferred embodiment, the modified tetracycline repressor, operator, and/or repressor/operator cassette of the present invention may be used to modulate gene expression of any gene in a plant, and/or other organism.

Moreover, the polynucleotide sequence of interest may be a polynucleotide sequence encoding a gene, an antisense polynucleotide, a ribozyme, a fusion protein, a polynucleotide encoding an antibody, etc. In specific embodiments, the polynucleotide sequence may a polynucleotide encoding a plant hormone, plant defense protein, a nutrient transport protein, a biotic association protein, any gene in an antisense orientation, etc.

Also provided is a method of producing herbicide resistant plants using the modified tetracycline repressor, operator, and/or repressor/operator cassette of the present invention to control the expression of a herbicide resistance gene, for example.

### tet Receptor Cassette: Effect of Modification with NLS and Expression from Different Promoters

tet Receptor fused to the SV40 Large T-antigen Nuclear Localization Sequence (nTR) provided for much stronger repression of an inducible reporter gene in transient assays. nTR driven by 35S promoter showed the strongest repression of the reporter gene in leaves and, especially, in meristems of tobacco plants.

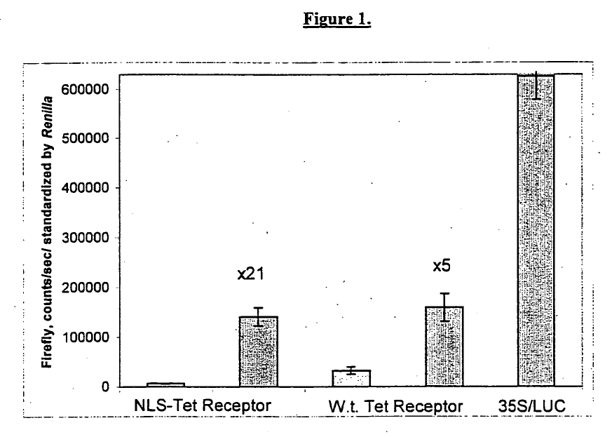

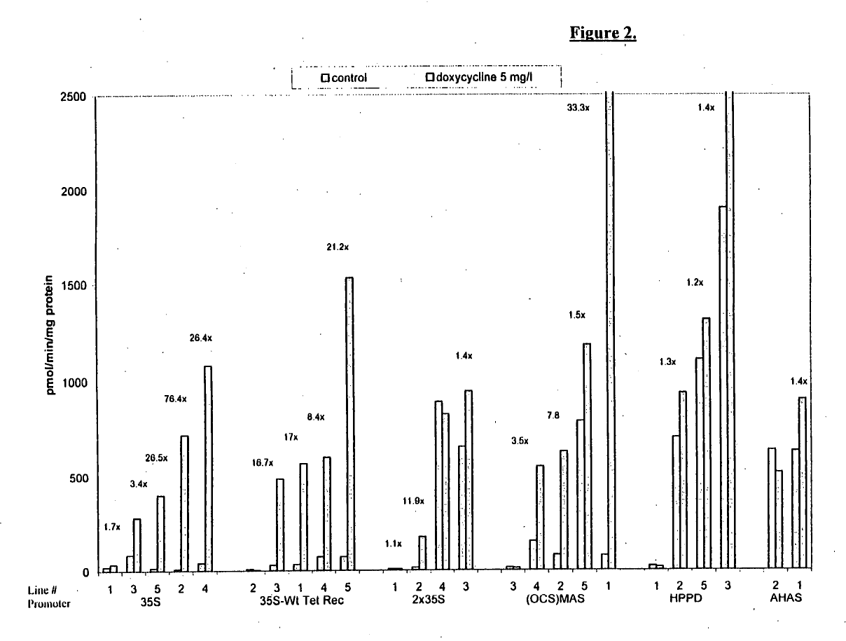

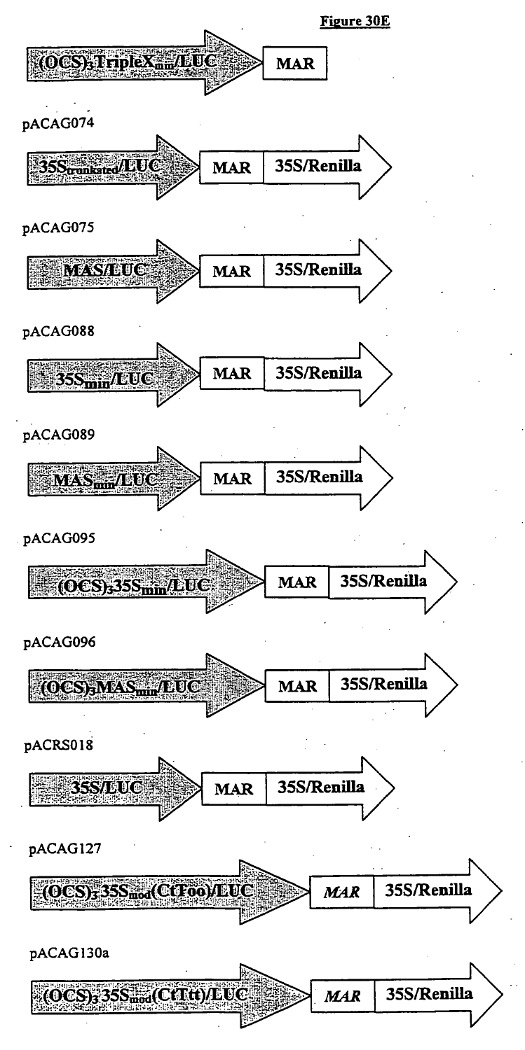

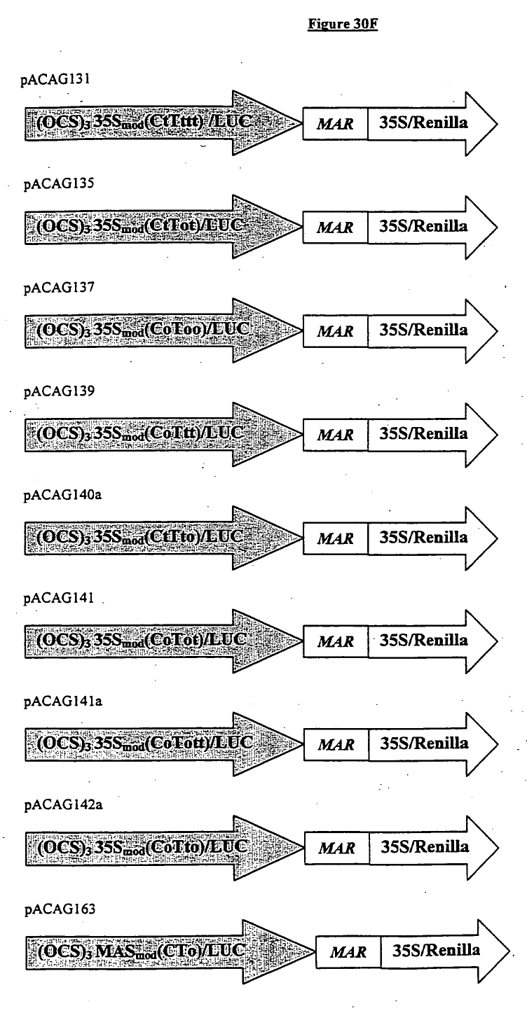

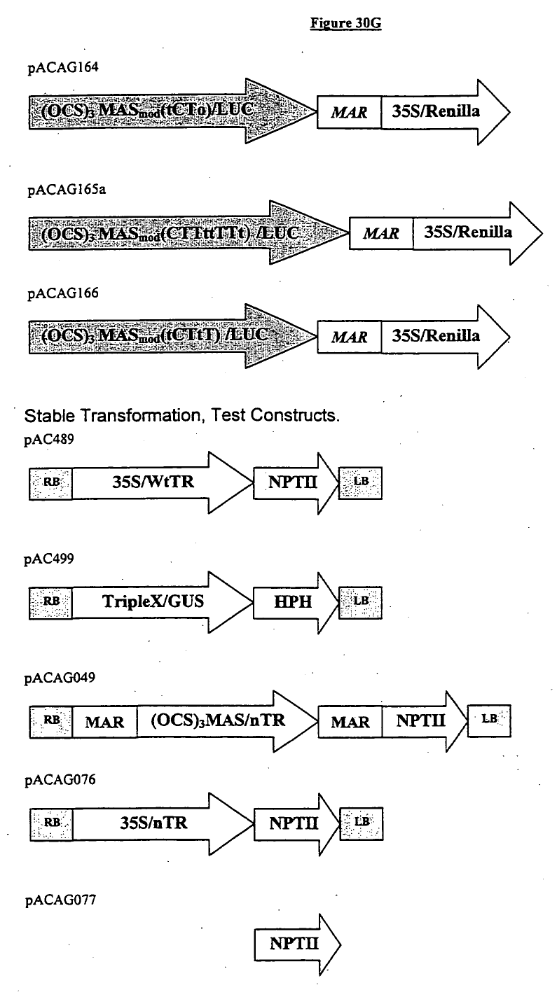

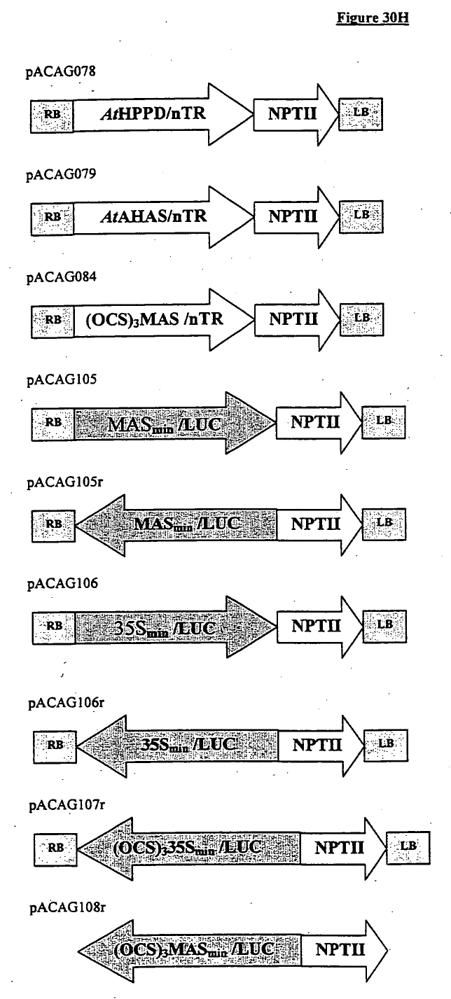

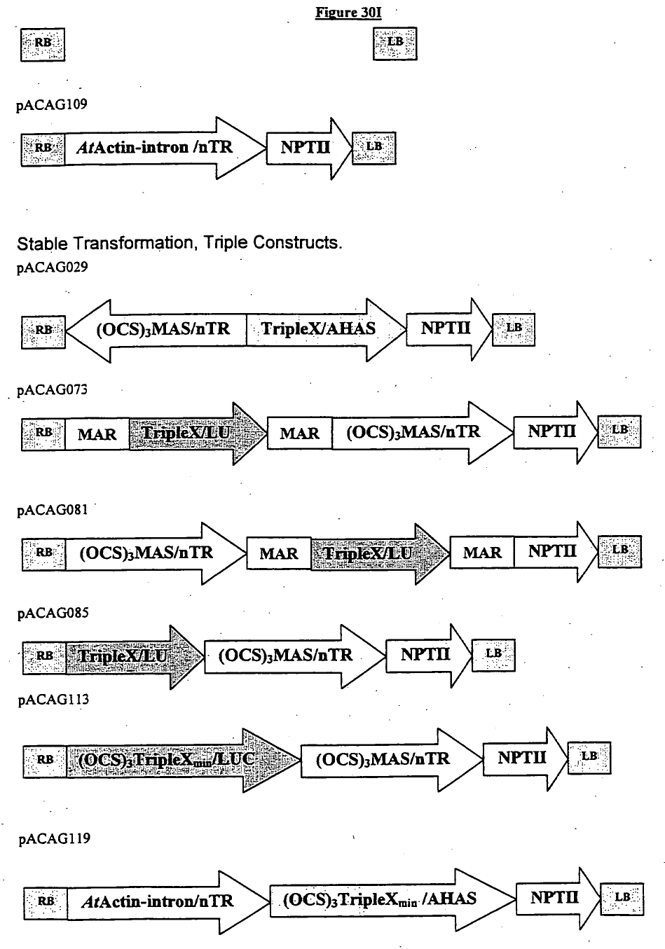

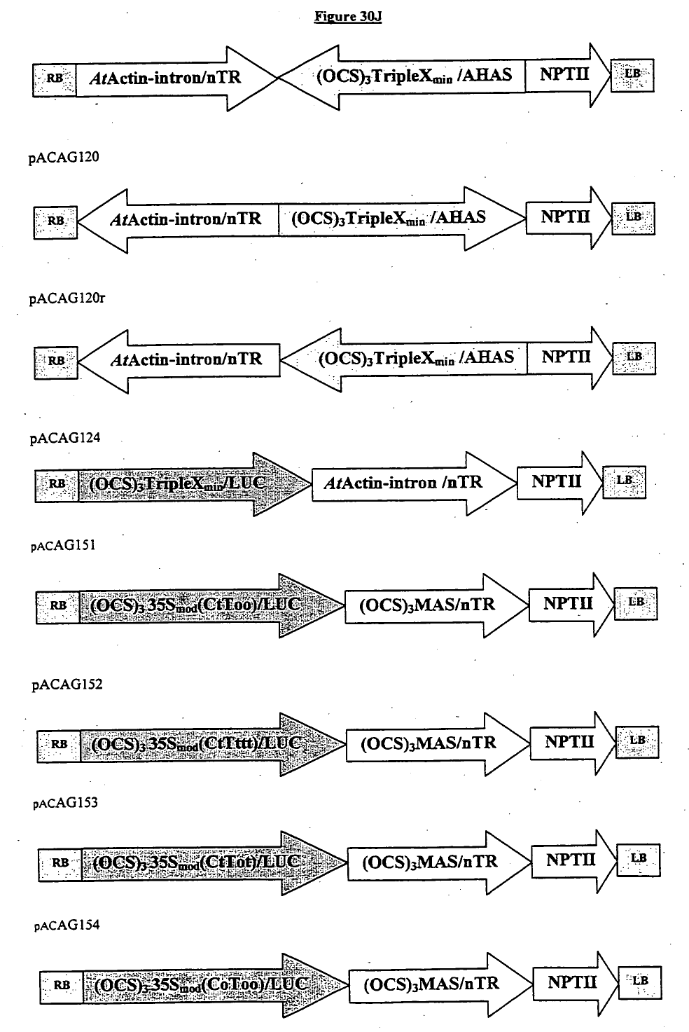

Modified tet Repressor and TripleX promoter were tested in transient assays to make sure the system was working, prior to performing stable transformations. TripleX was inserted into a promoter testing plasmid such that it drives the Firefly luciferase reporter gene (pACAG024, see FIGS. 30A-K). The other reporter gene on the pACAG024 cassette, Renilla luciferase, was intended to act as an internal control and was placed under control of the constitutive 35S promoter. The MAR element was inserted between the genes to act as an insulator to reduce interaction of the two genes especially transcriptional read-through from the Firefly luciferase gene. Firefly and Renilla luciferase activities were assayed after co-electroporation of NT1 cells with 20 μg of pACAG024 and 15 μg of either pACAG015 ((OCS)₃MAS/nTR) or pACAG016 ((OCS)₃MAS/WtTR) followed by cultivation of one half of the electroporated cells with 3 mg/l of tetracycline, and the other half without inducer. Electroporation with pACRS018 (35S/LUC, see FIGS. 30A-K) was performed for control purposes. FIG. 1 shows results of Firefly luciferase assay. As shown in the figure, tet Receptor fused to the Nuclear Localization Sequence provides for more than 4-fold lower background expression (i.e. stronger repression) of luciferase gene than the wild-type tetracycline repressor cassette. On the other hand, induced expression of the reporter from the TripleX promoter was several fold lower than the expression observed from the 35S promoter, the predecessor of the TripleX. This could be explained by incomplete derepression of the reporter gene and changes in promoter's sequence.

After obtaining encouraging results in transient assays, the nTR coding region was used to construct vectors for plant transformation. In the course of further development, the tissue-specific performance of the modified nTR chemical switch was investigated using a number of available promoters. Five pCAMBIA-based Agro cassettes were made carrying NPTII marker gene and nTR under control of 35S, 2×35S, A. thaliana HPPD, A. thaliana AHAS, A. thaliana Actin or (OCS)₃MAS promoters (pACAG076, 077, 078, 079, 109 and 084, see FIGS. 30A-K). Appropriate transferred Agro strains were created and used for transformation of tobacco already expressing pAC499 (TripleX/GUS, see FIGS. 30A-K). For control purposes, pAC499 tobacco was also transformed with pAC489 (35S/WtTR, see FIGS. 3OA-K). Transformation of tobacco produced a number of Kanamycin-resistant shoots for all cassettes, though only 4-5 plants developed roots under selection pressure after 25 lines per each cassette were isolated and transferred to rooting medium. Preliminary analysis using histochemical GUS assays (see Table 1) showed that background expression (in the absence of inducer) was negligible in leaves and low in roots in lines transformed with cassettes where nTR was driven by 35S or 2×35S promoters and high for other cassettes indicating strong expression of nTR in these cassettes. These results confirm the leaf-specific pattern of the 35S promoter. Also, by comparing the expression of wild type and NLS-modified tet Receptors, it was possible to note the stronger repression of the GUS reporter gene, with the latter in both leaves and roots.

TABLE 1
Histochemical analysis of leaking expression of GUS
gene in plants transformed with TripleX/GUS and
promoter/nTR cassettes.

| Promoter | Tissue | Background |
|---|---|---|
| 35S/WtTR | Leaves | Moderate |
| | Roots | Moderate |
| 35S/nTR | Leaves | No |
| | Roots | Low |
| 2 × 35S/nTR | Leaves | No |
| | Roots | Low |
| (OCS)₃MAS/nTR | Leaves | Moderate |
| | Roots | Moderate |
| HPPD/nTR | Leaves | High |
| | Roots | High |
| AHAS/nTR | Leaves | High |
| | Roots | High |

TABLE 2
Histochemical analysis of leaking expression of GUS
gene in plants transformed with TripleX/GUS
and nTR cassettes.

| Cassettes | Tissue | Background |
|---|---|---|
| (OCS)₃MAS/nTR | Leaves | Moderate |
| | Roots | Moderate |
| MAR-(OCS)₃MAS/nTR | Leaves | Moderate |
| | Roots | Low |

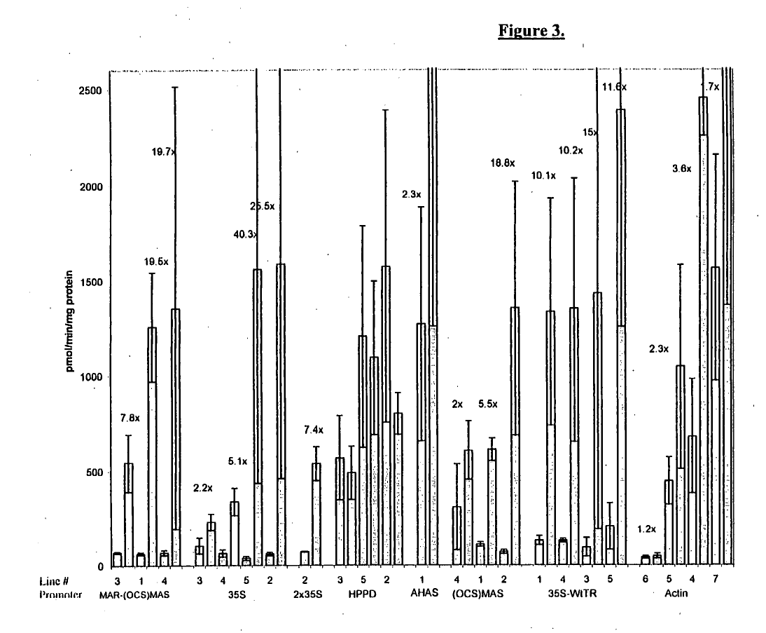

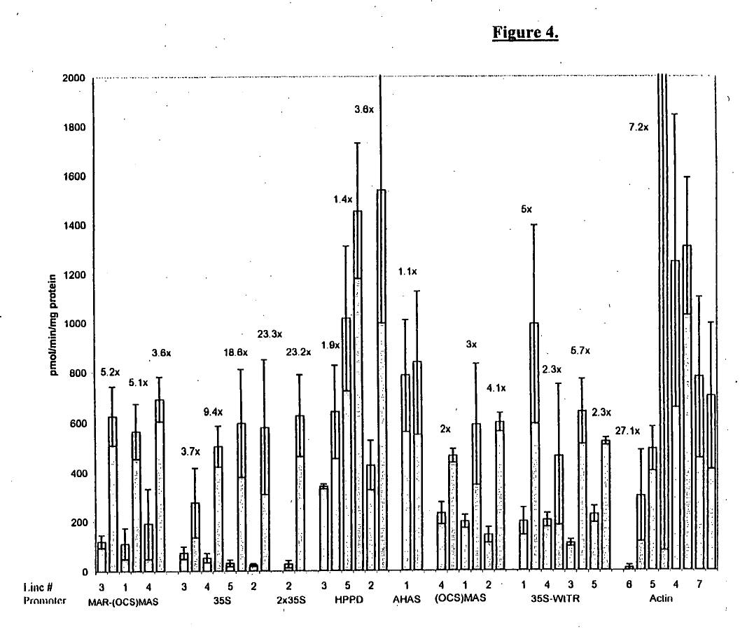

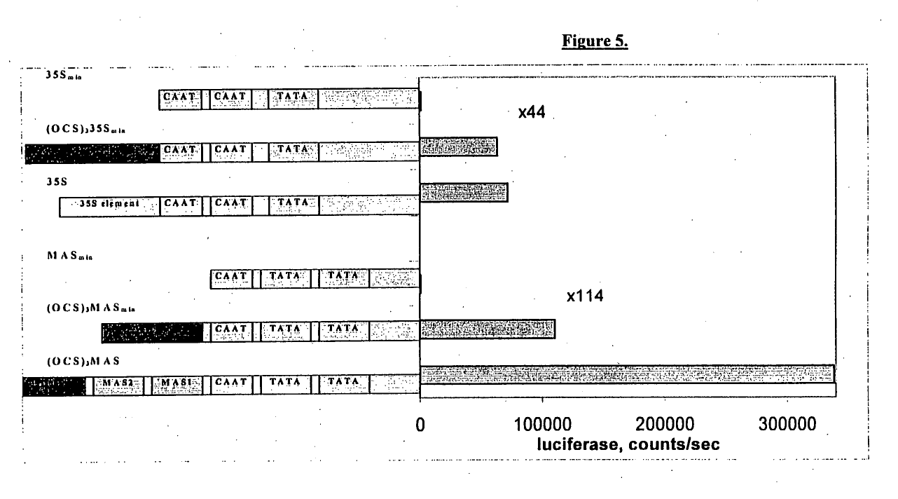

This collection of tobacco plants was tested in preliminary induction experiments performed on leaf disks. Tissues were induced with Doxy 5 mg/l. Results of GUS assays are presented in FIG. 2. Not all lines selected for Kanamycin resistance showed induction of the reporter gene. As was shown in histological assays, the NLS-modified tet Receptor provided stronger repression of reporter than wild type receptor (as indicated by lower GUS gene expression in uninduced tissues), but lower level of expression in induced tissues. nTR driven by 35S promoter was the best effector of those tested, as it provided the lowest average background expression and highest average magnitude of induction (26 to 76 fold). (OCS)₃MAS promoter did not express nTR strong enough to repress the GUS activity in leaves, as compared to the 35S promoter. HPPD and AHAS promoters showed the highest background expression and weak induction (1.4 fold maximum). Lines that showed induction in preliminary experiments and some AtActin/nTR lines were taken for advanced analysis. Leaf disks and pieces of meristems and roots were excised from plants and used in a test similar to previously described leaf disk test. In this experiment, tissues were induced with Doxy 5 mg/l. Results of the GUS assays are presented in FIGS. 3 and 4 (leaves and meristems respectively). As shown in previous experiments, the NLS-modified tet Receptor provided the strongest repression of reporter than wild type receptor (as indicated by lower GUS gene expression in uninduced tissues), especially in meristem. This finding was very important because, according to previous experiments with the wild type tet Receptor, the leaky expression of the reporter was the strongest in apical meristems. (OCS)₃MAS promoter did not express nTR strong enough to repress the GUS activity in leaves, as compared to the 35S promoter. HPPD, AHAS, and, surprisingly, Actin promoters showed the highest background expression and weakest levels of induction.

### Improvement of Tet-Inducible Promoters with OCS Elements

Even though OCS elements proved to restore expression of a reporter gene if placed upstream of a minimal promoter, this effect was not observed in stable transformations of tobacco. Instead, the tissue specificity changed depending upon the orientation of the cassette in which a minimal promoter was present. Replacement of the upstream activator sequence in the wild-type TripleX promoter (tet-inducible 35S promoter) with three OCS elements resulted in a significantly higher magnitude of induction of the reporter gene driven by the chimeric promoter in transient assays. Similar effects were observed in stable transformants, though the difference was not as dramatic as in transient assays.

Two minimal promoters, 35Sₘᵢₙ and MASₘᵢₙ, were designed (see FIGS. 30A-K to compare these promoters with the original ones), produced by PCR and cloned into promoter testing vectors upstream of luciferase gene (pACAG088 and 089, see FIGS. 30A-K). These cassettes were used in quick transient assays and showed very low levels of expression. In order to test the possibility of restoring activities of minimal promoters by addition of upstream activating elements, cassettes carrying the 35S minimal and MAS minimal promoters with OCS elements placed at the 5′ end were made ((OCS)₃35Sₘᵢₙ and (OCS)₃MASₘᵢₙ, see FIGS. 30A-K). These promoters were placed upstream of the luciferase gene in a high copy number vector for transient expression (pACAG095 and 096, see FIGS. 30A-K) and used in transient assays with NT1 cells along with cassettes with original wild type and minimal promoters. Results shown in FIG. 5 indicate that OCS elements restore transcription from both promoters. Moreover, expression from (OCS)₃35Sₘᵢₙ promoter was about the same as that of the original wild type 35S promoter. Therefore, a single OCS element is in fact a much weaker enhancer than an upstream piece of 35S promoter. Also, expression from the original (OCS)₃MAS was twice as high as that of the (OCS)₃MASₘᵢₙ—implying that the MAS elements are important, but not a critical part of the original promoter.

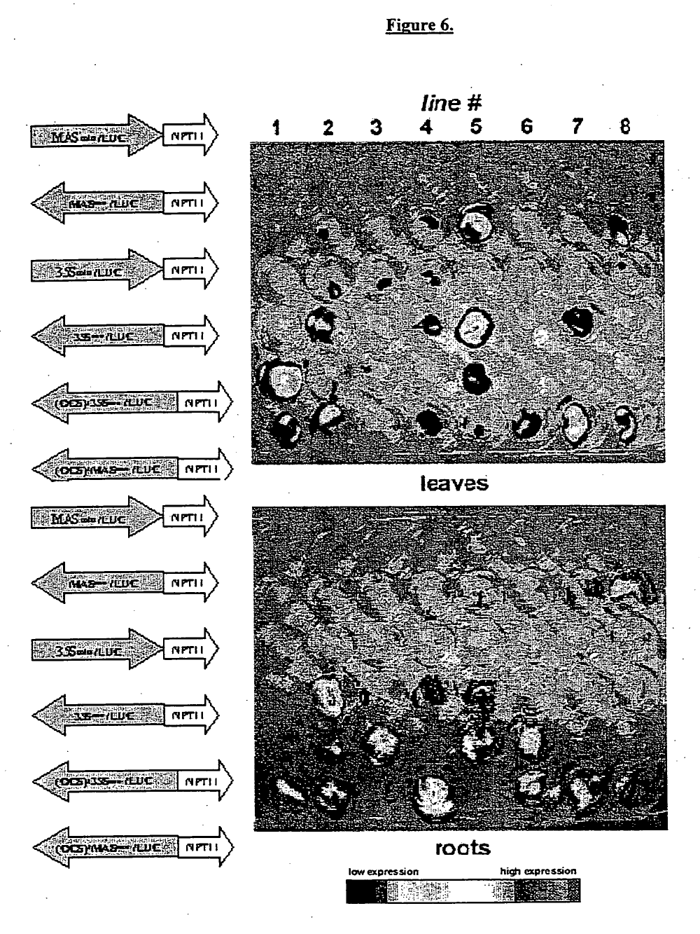

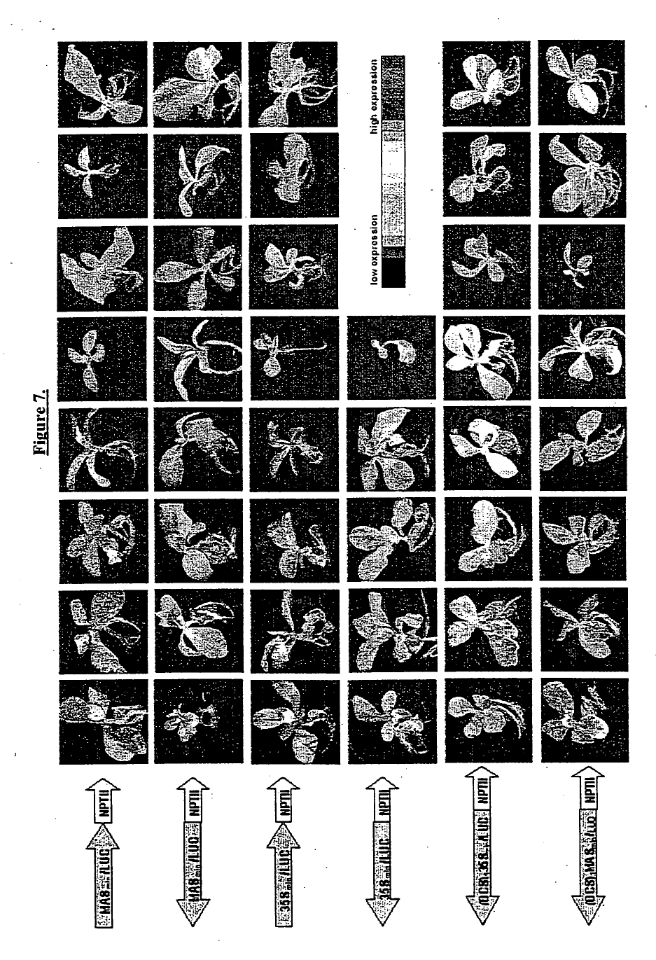

Results of transient assays raised several other questions: How would minimal and new chimeric promoters work on a whole plant level? Does tissue specificity change when MAS elements are removed from (OCS)₃MAS? In order to address these questions, the genes carrying luciferase driven by either minimal or chimeric promoters were cloned into Agro vectors to create cassettes pACAG105 through 108 (see FIGS. 30A-K). The resulting cassettes were transformed into Agro strain LBA4404 and used in transformations of wild type tobacco and Arabidopsis. Later, a collection of transgenic tobacco plants carrying (pACAG105-108) were created. Root and leaf tissue samples from these plants were imaged in a Night Owl luminometer after treatment with luciferase. Results are shown in FIG. 6. The MAS minimal promoter did not produce any expression in any tissue when its cassette was oriented such that the promoter was near the transcriptional border (pACAG105, see FIGS. 30A-K). On the other hand, when the minimal promoter was placed next to the upstream sequence of the 35S promoter driving the NPTII gene (pACAG105r, see FIGS. 30A-K), several plants showed luciferase expression. Almost the same was true for the 35S minimal promoter. However, even pACAG106 showed some activity in leaves. This result could be explained by possible localization of some activating sequences close to the T-DNA insertion point. OCS elements placed upstream of both minimal promoters increased expression of the luciferase gene significantly; expression of the reporter from (OCS)₃35Sₘᵢₙ promoter was equally strong in both leaves and roots, while (OCS)₃MASₘᵢₙ expressed better in roots, just like the original (OCS)₃MAS promoter. This finding indicates that MAS upstream activating sequences of the original (OCS)₃MAS promoter, do not influence the tissue specificity of the promoter expression.

The same tobacco plants transformed with one of the pACAG105 through 108 cassettes were imaged in the Night Owl after treatment with luciferase. Images were collated in FIG. 7. Unlike the results observed in transient assays (where imaged tissue samples transfected by minimal promoters expressed much lower than the same ones enhanced with OCS elements), this experiment showed no significant difference between expression of enhanced and minimal promoters. Plants transformed with each cassette showed different levels of expression, from almost no expression to very high expression throughout all plant organs observed. Consistently, in those cassettes in which the luciferase gene was driven by the minimal promoters placed in an orientation opposite to that of the NPTII gene (pACAG105r and 106r), a much stronger expression in roots were observed, while forward orientations produced high expression in leaves. This was true for both the MASₘᵢₙ and 35Sₘᵢₙ promoters.

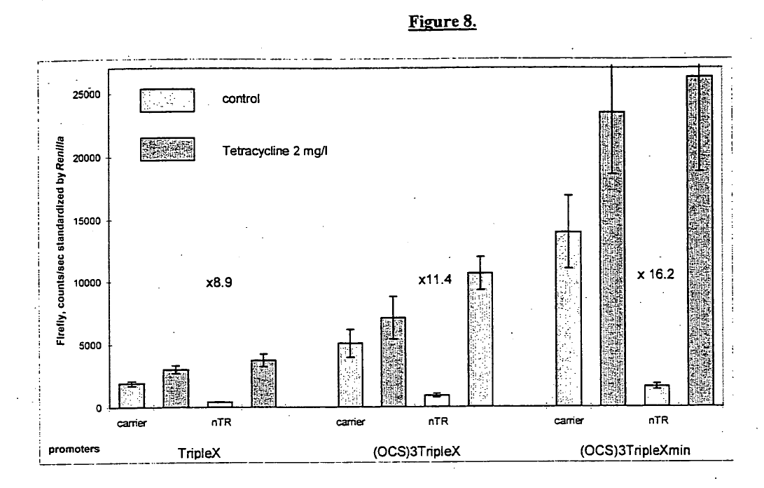

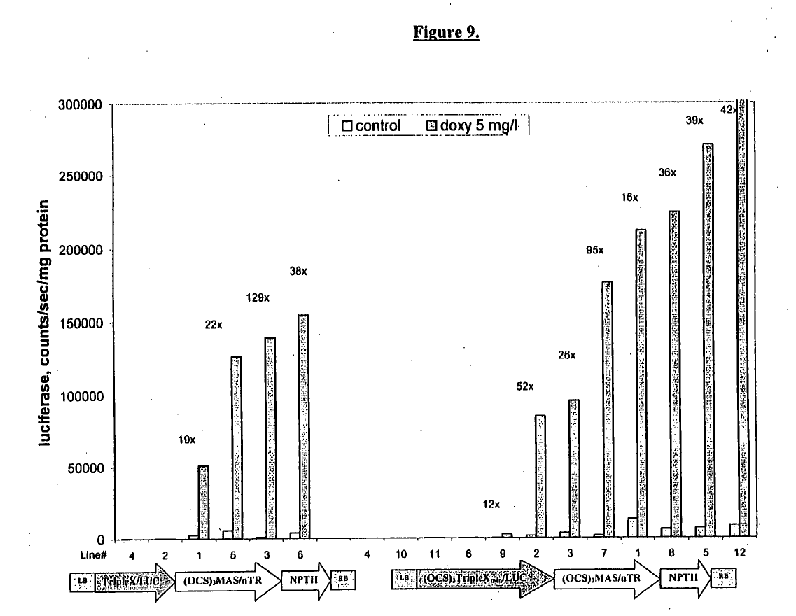

In order to construct a tet-inducible promoter that would provide higher expression of the reporter gene, OCS elements from (OCS)₃MAS promoter were fused to the TripleX promoter (both wild-type and minimal) to yield (OCS)₃TripleX and (OCS)₃TripleXₘᵢₙ respectively (see FIGS. 30A-K). The minimal Triplex promoter was defined as a 75 bp sequence that included only original CAAT and TATA boxes, three tet operators, and a small 25 bp fragment (from the 35S promoter element) upstream of the CAAT box. These promoters were fused to the luciferase gene to yield pACAG042 & pACAG050 (see FIGS. 30A-K) and subjected to transient expression experiments. In control experiments (with reporter cassettes only), a plasmid similar in size to the nTR cassette (e.g., pACAG013) was used to offset the effect of increased expression caused by increased DNA sample with addition of the tet Receptor cassette in test experiments. Results are presented in FIG. 8. The OCS elements increased expression of TripleX promoter in both cassettes; 2-fold increase was achieved by (OCS)₃TripleX and more than 7-fold—by (OCS)₃TripleXₘᵢₙ. Even though the leaky expression of luciferase driven by the (OCS)₃TripleX and (OCS)₃TripleXₘᵢₙ promoters was almost twice as high as that of original wild-type cassette, the amplitude of derepression (i.e., induction) also increased (from 8.9-fold for Triplex to 16.2-fold for (OCS)₃TripleXₘᵢₙ). These results reinforced previous findings that the OCS elements have a positive effect on gene expression from a promoter.

In order to test the performance modified Triplex promoter containing OCS elements on the whole plant level, the cassette consisting of (OCS)₃TripleXₘᵢₙ/LUC gene was cloned into pCAMBIA-based Agro vector carrying (OCS)₃MAS/nTR and NPTII marker genes (pACAG113, see FIGS. 30A-K). For control purposes, another cassette carrying TripleX/LUC was cloned into the same vector (pACAG085, see FIGS. 30A-K). These cassettes were transformed into Agro and resulting strains were used for transformation of the wild type tobacco. The transformation produced a number of transgenic plants. These plants were tested in leaf disk induction assays with doxycycline 5 mg/l. Results of the assays presented in FIG. 9 indicated strong induction of reporter gene in both cassettes. The cassettes showed an average 30 to 40 fold induction of the reporter, with some lines showing induction as high as 100 fold. Comparing these cassettes, it was possible to observe stronger induction and higher background levels of luciferase expressed from the (OCS)₃TripleXₘᵢₙ promoter and TripleX promoter, respectively. Therefore, similar to the results observed in transient assays, OCS elements enhanced gene expression of the promoter, though at a lower magnitude (in transient assays OCS elements increased expression almost 7-fold).

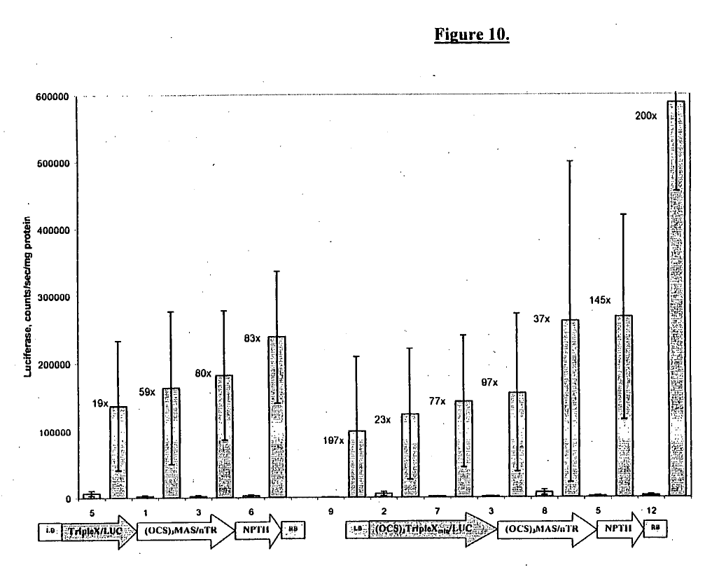

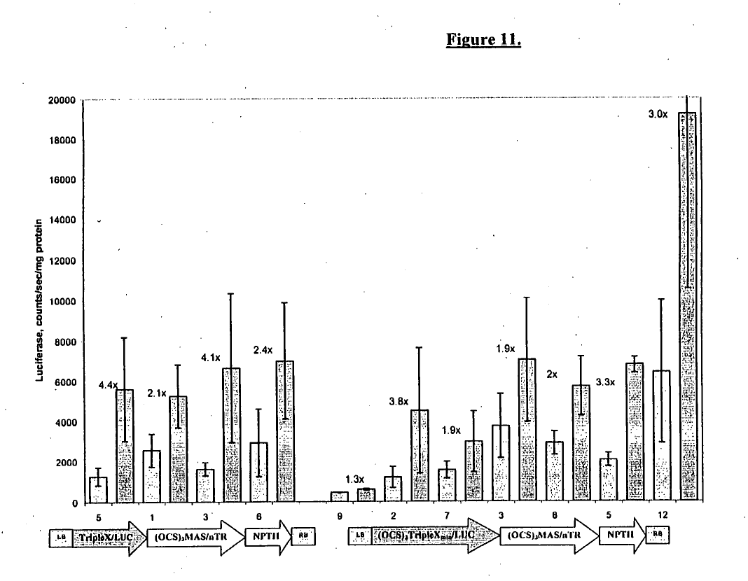

Lines that showed induction in preliminary experiments were taken for advanced analysis. Leaf disks and pieces of roots were excised from plants and used in a test similar to the previously described leaf disk test. In this experiment tissues were induced with Doxy 5 mg/l. Results of the luciferase assays are presented in FIGS. 10 and 11 (leaves and roots respectively). Comparing results for roots and leaves it was easy to note the difference in the magnitude of reporter gene induction between leaves and roots. The weak derepression of the system in roots could be explained by ineffective uptake of the tet ligand (i.e., inducing agent) by root tissues. As shown in previous experiments, the average luciferase expression was stronger when it was driven by (OCS)₃TripleXₘᵢₙ promoter than the original wild-type TripleX. The other important observation was that, unlike the transient assay results, leaky expression of uninduced leaf disks was on average lower for the (OCS)₃TripleXₘᵢₙ promoter than for the cassette with the original wild-type TripleX promoter. As a result, the magnitude of induction for the pACAG113 cassette was more than higher than 100-fold higher for several lines, compared to a maximum of 83-fold induction for pACAG085. Also, there was a correlation between expression of luciferase in different tissues: lines that showed strong induced expression in leaves also showed higher levels of luciferase expression in roots, and vise versa.

### Effect of MAR Elements on Performance of tet-Inducible System

The addition of at least one MAR element showed a strong positive effect on expression of effector gene in double transformants: nTR gene flanked with at least one MAR element repressed the reporter gene stronger than the same cassette without at least one MAR element. However, using the MAR element-flanked reporter gene along with the tet repressor on the same cassette produced only moderate increases in the level of both leaky and induced expression, which resulted in lower magnitudes of gene induction.

In the course of making the nTR cassette for plant transformation, the NLS-tet Repressor coding region was placed under the control of the (OCS)₃MAS promoter (pACAG084, see FIGS. 30A-K). The other cassette placed the NLS tet repressor coding region under the control of the (OCS)₃MAS/nTR promoter placed between two 1100 bp MAR elements (pACAG049, see FIGS. 30A-K). Appropriate Agro strains transformed with these cassettes were created and used for transformation of tobacco already expressing pAC499 (TripleX/GUS and HPH genes, see FIGS. 30A-K). Transformation of tobacco produced a number of Kanamycin-resistant shoots for all cassettes. 25 lines per cassettes were isolated and transferred to rooting medium. Of these 25, roughly 4-5 plants developed roots under Kanamycin selection pressure. Preliminary analysis using histochemical GUS assays (see Table 2) showed that the nTR gene flanked with MAR elements repressed the GUS gene in roots stronger than the same gene without the MAR elements.

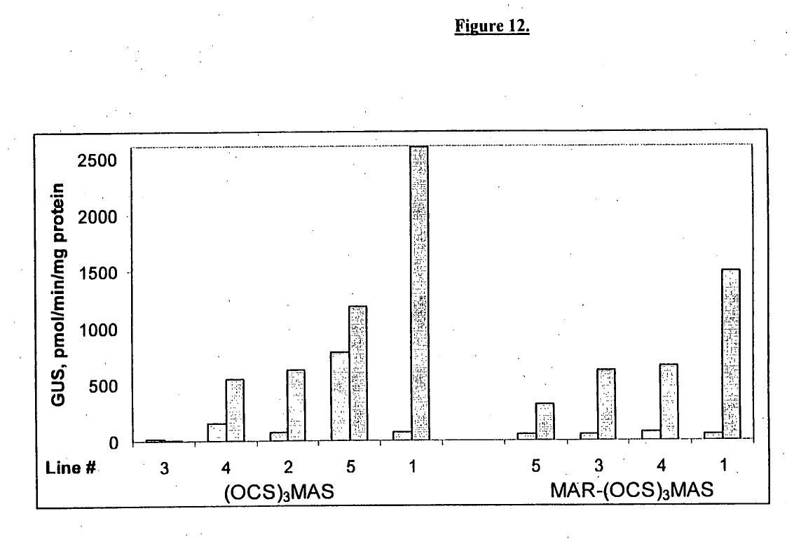

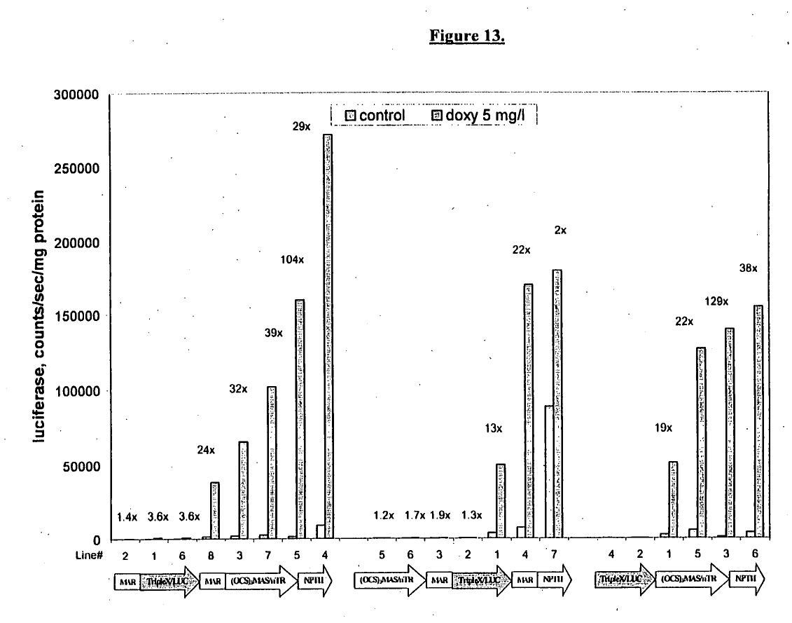

This collection of tobacco plants was tested in a preliminary induction experiment performed on leaf disks. Tissues were induced with Doxy 5 mg/l. Results of GUS assays are presented in FIG. 12. Not all Kanamycin resistant lines showed induction of the reporter gene. As shown in histological assays, (OCS)₃MAS promoter did not express nTR strong enough to completely repress the GUS activity in leaves, but this problem was partially solved in the cassette where the (OCS)₃MAS/nTR promoter was placed between the two MAR elements: the variation in background expression among different lines was significantly smaller for the MAR-(OCS)₃MAS cassette.

Lines that showed induction in preliminary experiments were taken for advanced analysis. Leaf disks and pieces of meristems and roots were excised from plants and used in a test similar to the previously described leaf disk test. In this experiment, tissues were induced with Doxy 5 mg/l. Results of the GUS assays are presented in FIGS. 3 and 4 (leaves and meristems only). The (OCS)₃MAS promoter did not express nTR strong enough to completely repress the GUS activity in leaves, but the leaky expression dropped twice as much, on average, when the cassette was placed between the two MAR elements.

After encouraging results were observed with the effector cassette (e.g., pACAG049) placed between the MAR elements, subsequent experiments were directed to determine the effect of placing an inducible gene between the same MAR elements, as well. As a result, several cassettes were made in order to evaluate the effect of MAR elements on the expression of a tet-inducible reporter. In this experiment, the cassette carrying tet Receptor was also cloned into the vector. TripleX-driven luciferase gene placed between MAR elements was cloned into an Agro vector carrying (OCS)₃MAS/nTR and NPTII marker genes to yield vector pACAG073 (see FIGS. 30A-K); pACAG081 carried the same genes as pACAG073, but the MAR-flanked TripleX-driven luciferase gene was placed in between the (OCS)₃MAS/nTR and NPTII cassettes (see FIGS. 30A-K). pACAG085 was created as a reference cassette which carried aforementioned genes without a MAR element (see FIGS. 30A-K). All these vectors containing cassette of the present invention were transformed into Agro LBA4404 and resulting strains were used for transformation of wild type tobacco.

The transformation produced a collection of transgenic plants. Leaf disks were cut from these plants and cultivated in liquid medium with or without doxycycline 5 mg/l for five days. Samples were assayed for luciferase activity. Results of these assays, presented in FIG. 13, indicate strong induction of reporter gene in all of the cassette tested. The cassette with the MAR-flanked luciferase gene placed between the (OCS)₃MAS/nTR and NPTII cassettes produced a very low number of plants that responded to the application of tet inducer—with the strongest induction of 22 fold shown by only one line. The other three cassettes showed an average 30 to 40 fold induction of the reporter with some lines as high as 100 fold. Comparing these three cassettes, it was not possible to detect a difference in the level of expression/induction of reporter between vectors with or without one or more MAR elements. This observation could be explained by the fact that both effector and reporter genes were on the same vector with one or more MAR elements, and expression of both was enhanced by these elements, but the combined effect was not changed under repression nor induction conditions.

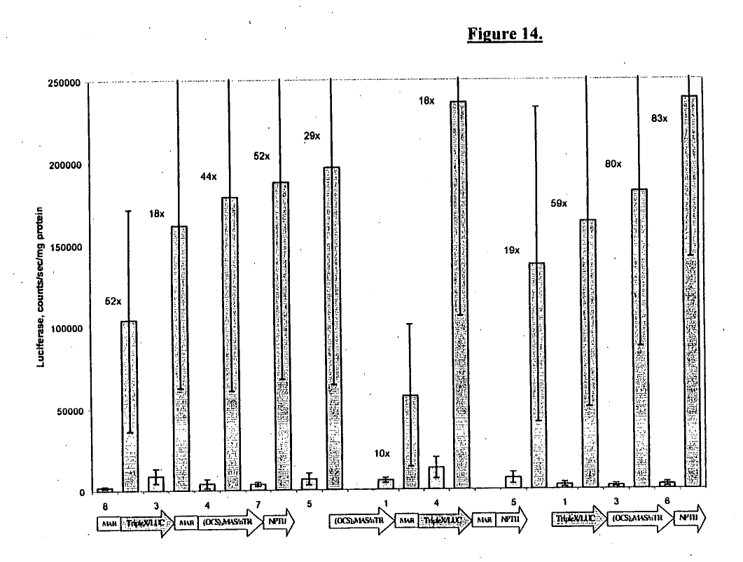

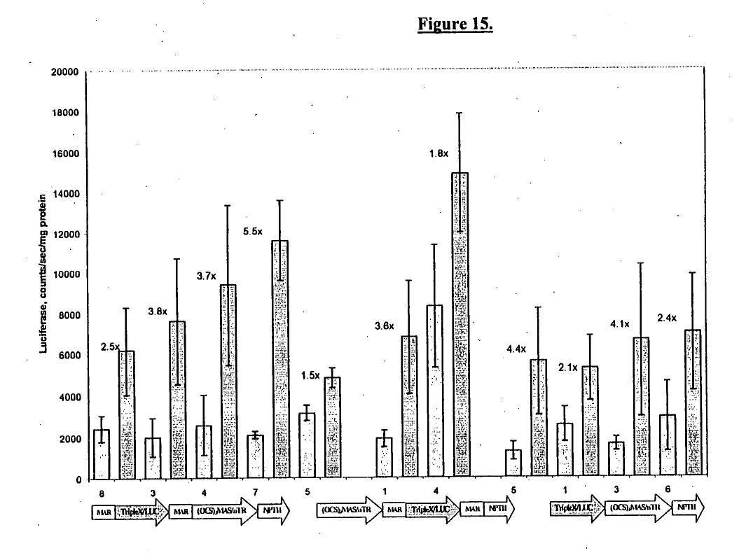

Lines that showed induction in preliminary experiments were taken for advanced analysis. Leaf disks and pieces of roots were excised from plants and used in a test similar to the previously described leaf disk test. In this experiment, tissues were induced with Doxy 5 mg/l. Results of the luciferase assays are presented in FIGS. 14 and 15 (leaves and roots respectively). Comparing the results observed for roots and leaves, it was possible to note the difference in magnitude of induction of the reporter gene between leaves and roots. The weak derepression of the system in roots could be explained by ineffective uptake of the inducer by root tissues. As shown in previous experiments, on average, the induced expression of luciferase was a little stronger when it was flanked with MAR elements than when at least one MAR element was not in the cassette. On the other hand, this effect was accompanied by slightly higher leaky expression in uninduced tissues. These effects were more obvious in roots than in leaves, probably because of root-specificity of (OCS)₃MAS promoter driving nTR. As a result, the lower magnitude of induction was observed for cassettes carrying at least one MAR element with a maximum of 50-fold compared to 80-fold induction in cassettes without at least one MAR element. Also, there was a correlation between expression of luciferase in different tissues: lines that showed strong induced expression in leaves also showed higher level of luciferase in roots, and vise versa.

### Kinetics of tet-Inducible System Derepression

Studies on the kinetics of tet induction of the tet inducible cassettes of the present invention on the whole plant level showed that the derepression of a reporter gene reached its maximum 48 hours after application of tetracycline and lasts for several weeks after the ligand is removed from the system.

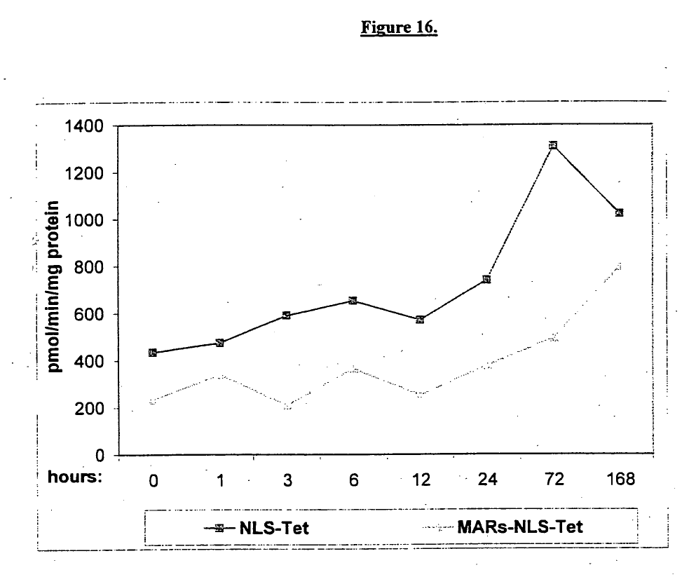

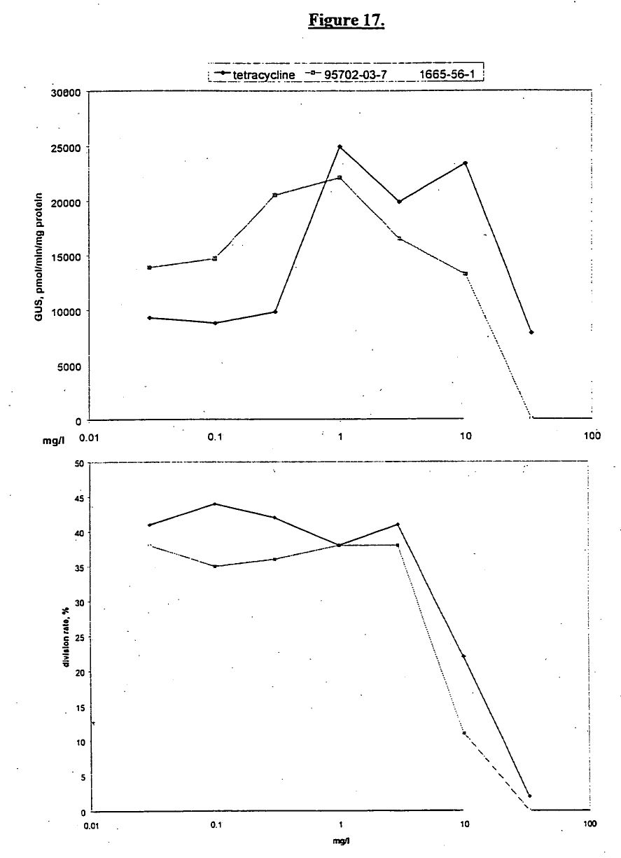

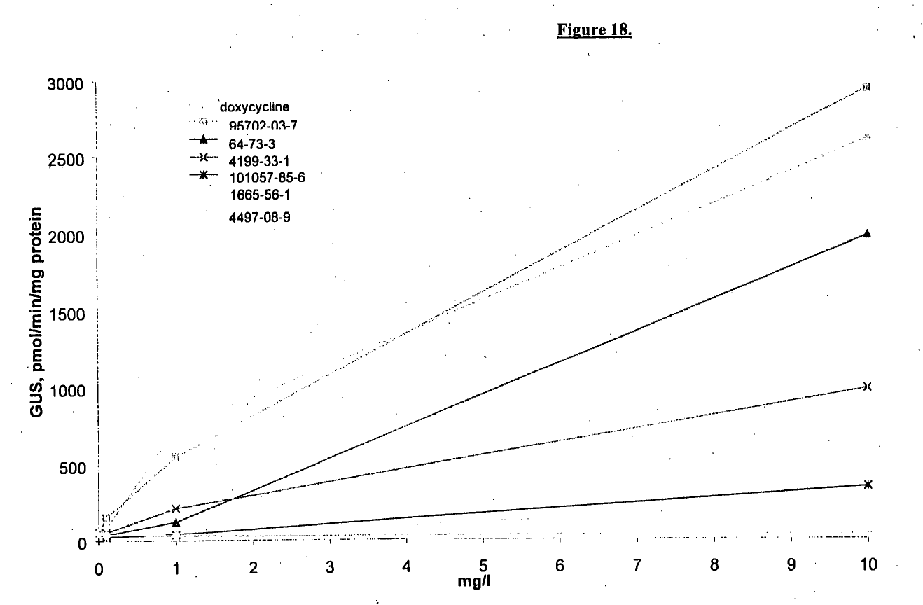

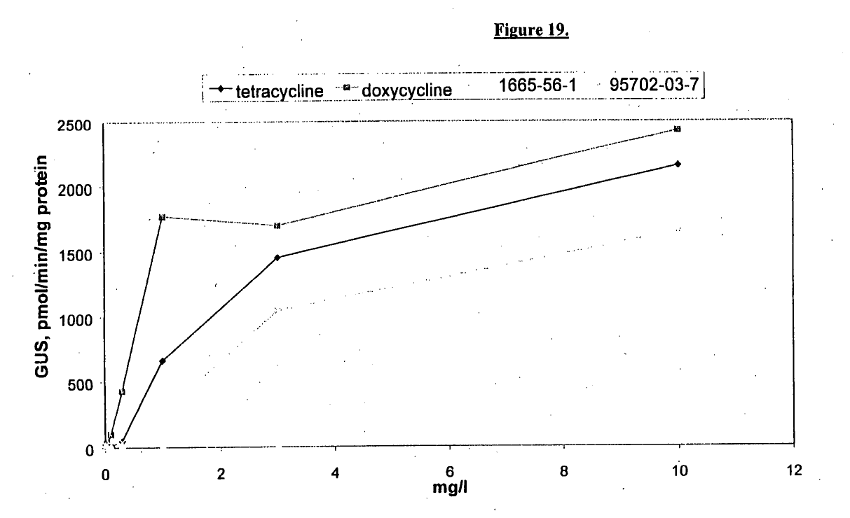

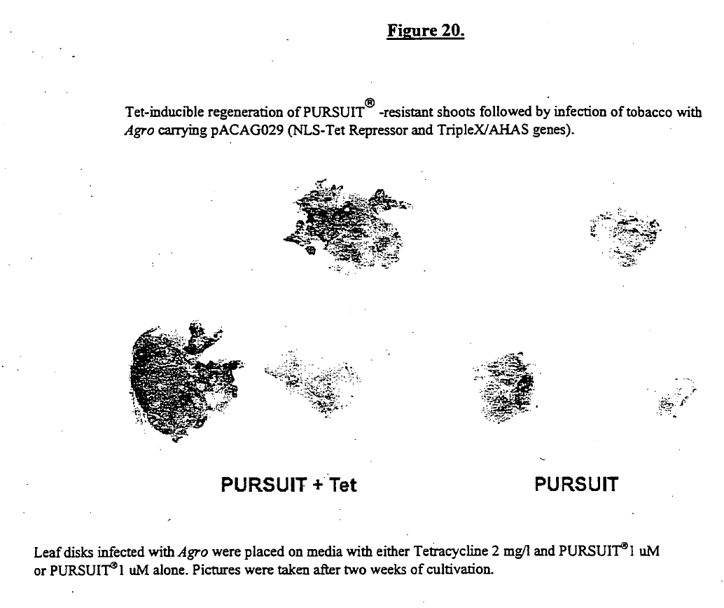

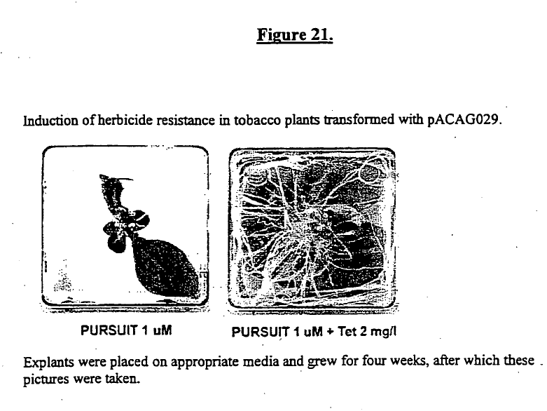

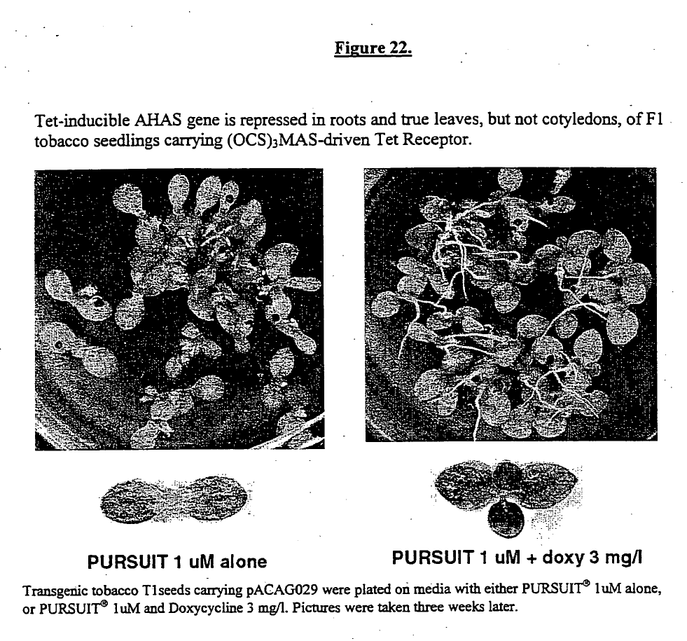

Time series were run with the best double transformants carrying pAC499 (TripleX/GUS) and either of pACAG084 ((OCS)₃MAS/nTR) or pACAG049 (MAR-(OCS)₃MAS/nTR). Three week-old rooted plants were transferred from agar to magenta boxes with liquid medium and tetracycline 2 mg/l. Tissue samples were regularly taken from leaves and roots and assayed for GUS. Unfortunately no induction of reporter was noticed in leaves; results for roots are presented in FIG. 16. Although the highest induction and highest background was observed by plants carrying nTR without at least one MAR element (confirming the previous observation of much stronger control of GUS gene expression by cassettes harboring at least one MAR element), the magnitude of induction was only 3.5-fold at maximum and it took more than one day before expression reached its peak. The latter result was consistently observed, as well as, in experiments from the literature.

### Testing Cyanamid's Proprietary Chemistry for Induction of the tet-Inducible System

95702-03-7 (6-deoxy, 6-demethyl tetracycline) showed very good induction of the GUS reporter gene among several tetracycline analogs chosen for in vitro tests. A couple of other analogs were determined to be moderate inducers.

Two analogs—95702-03-7 (6-deoxy-6-demethyl tetracycline) and 1665-56-1 (anhydrotetracycline)—as well as tetracycline were compared for their ability to induce the tet system in protoplast assays. Protoplasts were isolated from T2 transgenic tobacco carrying both wild type tet Receptor and GUS gene controlled by 35S promoter containing tet operators (pAC489 /pAC499 double transformants). Results are presented in FIG. 17. 1665-56-1 was found to be highly toxic to plant cells and not a good derepressing agent, as well. 95702-03-7 showed the same magnitude of derepression and toxicity as tetracycline, though it appeared to be a more potent inducer at concentrations of 0.3 mg/l and lower. According to the information provided by the supplier of the 95702-03-7 analog, such concentrations are not toxic to tetracycline-sensitive bacteria. This finding is very important in the light of tendency to reduce the field rate of toxic chemicals and lessen the impact on the environment.

After encouraging results in protoplast assays, an expanded number of analogs were tested in seed germination tests. Seeds of T2 homozygous tobacco carrying wild type tet Receptor and TripleX-driven GUS gene were germinated in the presence of several new analogs at different concentrations. Seedlings were visually evaluated for toxicity, collected and assayed. Visual evaluation showed that all analogs are non-toxic. Results of the GUS assays are shown in FIG. 18. High magnitudes of induction (32 to 48 fold) were achieved for doxycycline, 95702-03-7 (6-deoxy-6-demethyl tetracycline) and 64-73-3 (declomycin). It was surprising that the inducing activity of the 95702-03-7 analog was higher than that of the industry standard, doxycycline. 101057-85-6 (7-bromo-6-deoxy-6-demethyl tetracycline) was determined to be a moderate inducer and the rest of the analogs, including and 1665-56-1 showed negligible induction of the reporter gene. Recalling results protoplast assays where 1665-56-1 inhibited growth of plant protoplast but was able to induce the system, a conclusion might be drawn that this analog cannot penetrate the cell wall or is unstable under light. Also, comparing these two experiments, it appeared as though the level of GUS activity in experiments involving seedlings did not fade as the concentration of inducer reached a particular point, as was shown in protoplast assays. This could be explained by much higher sensitivity of protoplasts to toxic effects of chemicals.

The next step in evaluating the analogs was to determine their respective concentration curve. Seeds of homozygous tobacco carrying wild type tet Receptor and TripleX-driven GUS gene (pAC489/pAC499 double transformants) were germinated in the presence of several analogs at different concentrations. Seedlings were visually evaluated for toxicity, collected and assayed. Toxicity evaluation showed that only tetracycline and doxycycline slightly stunted growth of plantlets at concentration of 10 mg/l. Results of the GUS assays are shown in FIG. 19. The threshold concentration at which noticeable induction occurred was 0.1 mg/l for doxycycline and 1 mg/l for the other analogs. Unusually high magnitude of induction at maximum concentration was achieved for all analogs (80 to 120 fold) except for 1665-56-1 (anhydrotetracycline). Doxycycline provided the best induction; induction shown by 95702-03-7, though of lower magnitude, was very promising because this compound has lower toxicity than doxycycline and tetracycline and represents Cyanamid's proprietary chemistry.

In light of the encouraging results using a tetracycline inducible promoter cassette, the invention encompasses the application of any one of the modified tetracycline inducible promoter cassettes of the present invention, disclosed elsewhere herein, to the identification of novel tetracycline analogs and/or functional equivalents. For example, the method may be able to identify true tetracycline analogs, those compounds having structural similarity with tetracycline, that may have decreased toxicity to plant, bacterial, and/or animal tissues and cells, enhanced binding constants and/or binding kinetics (e.g., the analog may have a stronger affinity for tet repressor, or its equilibrium dissociation constant may be lower, thus requiring a lower concentration of such an analog to modulate gene expression), enhanced stability (e.g., thermal, environmental, photo, chemical, the compound may be chemically inert, etc.), etc. Alternatively, the method may be able to identify compounds capable of binding to the tet repressor, that are not characterized as a tetracycline analog, and that may share or differ in their mode of binding to the tet repressor (e.g., functional tetracycline equivalents).

The method of identifying novel tetracycline analogs and/or functional equivalents may comprise the following steps: i.) transfecting cells, plants, and/or tissues, stably or transiently, with a modified tetracycline inducible cassette of the present invention, ii.) applying a chemical compound to the transformed cells, tissues, and/or transgenic organism, and iii.) comparing the level of gene expression from the reporter gene in the modified tetracycline inducible cassette introduced in step “i.” between the chemical applied transformed cells, tissues, and/or transgenic organism to both a negative control and a tetracycline, and/or tetracycline analog, control. The skilled artisan would appreciate that step “i.” could be substituted by an acellular, or in vitro, system wherein all the necessary components for gene expression are present. Preferably, the method is a high throughput method (see Example 8).

### Application of tet-Inducible System to Produce tet-Inducible Herbicide Resistant Tobacco and Arabidopsis Plants

Several lines of tobacco and Arabidopsis transformed with a cassette carrying the NLS-tet Receptor, tet-inducible AHAS and NPTII marker genes showed tetracycline-inducible herbicide resistance. Induction of resistance to PURSUIT® was shown in original transformants (T0) as well as in T1 and T2 generations for both species. For Arabidopsis, T2 homozygous progeny showed stronger control over expression of AHAS gene in uninduced tissues.

Arabidopsis thaliana mutant AHAS gene was put under control of the TripleX promoter and OCS terminator (TripleX/AHAS, see FIGS. 30A-K). This cassette was cloned into an Agro vector carrying (OCS)₃MAS/nTR and NPTII cassettes to yield vector pACAG029 (see FIGS. 30A-K). The vector was transformed into Agro and resulting strains were used for transformation of both wild type tobacco and Arabidopsis.

As information about performance of different expression elements became available, the best promoters for selected for conferring inducible herbicide resistance. Arabidopsis Actin promoter with intron was fused to nTR coding region and (OCS)₃TripleXₘᵢₙ was fused to AHAS genes. The appropriate cassettes were cloned into an Agro vector carrying the NPTII marker gene yielding different orientations of the genes (pACAG119, 119r, 120 and 120r, see FIGS. 30A-K). The vector was transformed into Agro and resulting strains were used for transformation of both wild type tobacco and Arabidopsis.

Tobacco

During the first step of tobacco transformation with pACAG029, regeneration under selective pressure, three different selection schemes were created. Leaf disks infected with Agro were placed on three different media: Kanamycin-100 mg/l alone, to select all transgenic lines; tetracycline 2 mg/l and PURSUIT® 1 μM, to select lines with highest inducible herbicide resistance; and PURSUIT® 1 μM as a control for escape. In a couple of weeks, a number of Km-resistant shoots were observed and thirty of them were transferred to rooting medium. It took a little longer to regenerate shoots on media with herbicide, however these results showed that addition of tet induced regeneration of PURSUIT®-resistant tobacco (FIG. 20). Later, when these herbicide-resistant lines were separated and used individually in root induction assays, these lines appeared to be resistant to PURSUIT®even without tet inducer. 17 lines selected resistant to Kanamycin were checked for induction of PURSUIT® resistance on rooting medium containing either tet 2mg/l+PURSUIT® 1 μM or PURSUIT® 1 μM alone. Only four lines showed the induction: healthy plants with well-developed root systems grew on medium with tetracycline, whereas shoots growing on the medium with herbicide only were severely inhibited (FIG. 21).

These tobacco lines were transferred to soil for seed collection. As T1 seeds became available they were plated on media with either PURSUIT® 1 μM alone, or PURSUIT® 1 μM and Doxycycline 3 mg/l. All seed lines germinated on both media and produced green cotyledons, though after closer evaluation it was noted that roots were severely inhibited on seedlings of only one line, #4, growing on PURSUIT® alone compared to no root inhibition on the same media with Doxycycline (FIG. 22). Further evaluation revealed that true leaves were also inhibited on these seedlings (examples of the plantlets are shown in FIG. 22). Therefore, it can be concluded that (OCS)₃MAS promoter driving NLS-tet Receptor in these seedlings was not expressed in cotyledons, and that, in turn, led to expression of the AHAS gene and initial plant resistance. Later, when the plants developed roots and leaves, resistance disappeared as repressor started expressing in these tissues.

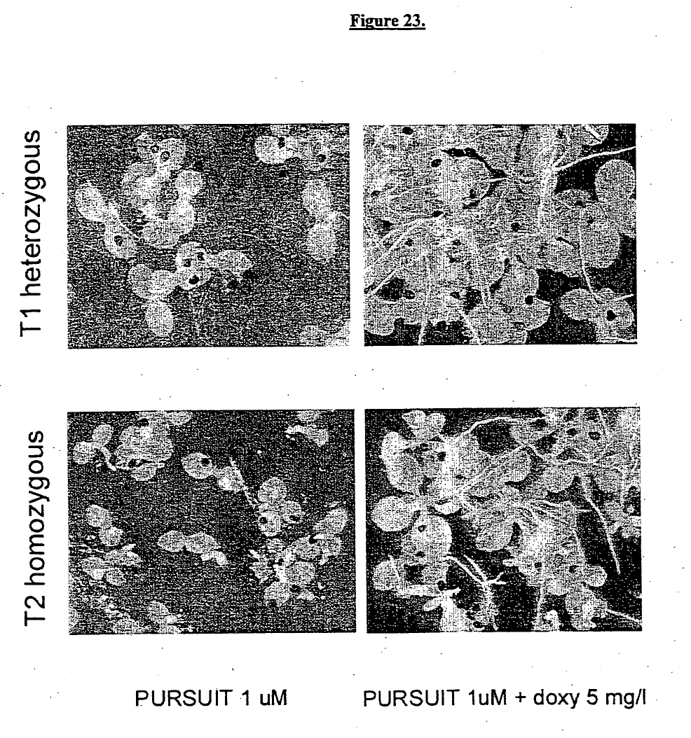

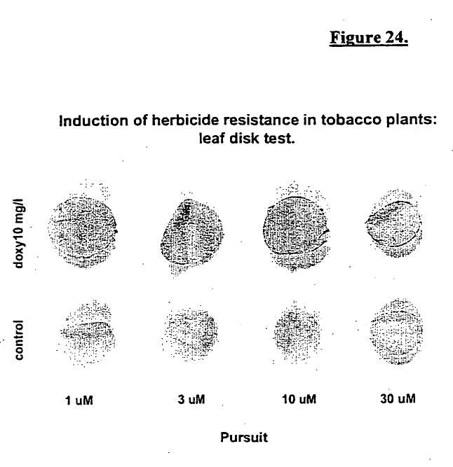

After T2 homozygous line of the transgenic tobacco carrying pACAG029 became available, the test was run to compare the induciblity of homozygous and heterozygous lines. T1 heterozygous and T2 homozygous seeds of the line were germinated on MS plates supplemented with 5 μM of PURSUIT® either alone or with doxycycline 5 mg/l. Results are shown in FIG. 23. Both T1 and T2 lines showed doxy-mediated herbicide resistance induction, though no significant differences between these lines were noted.

A concentration curve test was run using the tobacco line that showed the best tet-inducible herbicide resistance (pACAG029 #4). Leaf discs from T1 plants were floated on liquid medium supplemented with PURSUIT® 1, 3, 10 and 20 μM either alone or with doxycycline 10 mg/l. Three weeks later, the inhibition of tissues was evaluated (see FIG. 24). It was determined that herbicide concentrations of 20 μM was inhibitory to both induced and uninduced tissues, though the induced tissue was healthier. 1 μM of PURSUIT® did not completely inhibit the uninduced tissue—it retains some greenish color.

Almost 40 putatively transgenic tobacco lines were selected resistant to Kanamycin after transformation of wild type tobacco with pACAG119, 119r, 120, and 120r (AtAHAS under control of (OCS)₃TripleXₘᵢₙ promoter in a cassette with Actin-driven tet Receptor gene placed in different orientations relative to each other and to the third gene, NPTII marker). A test similar to the one used in drawing the concentration curve for tet-inducible transgenic line was used for determining lines that positively responded to the presence of a ligand. Leaf disks from tobacco plants under study and pACAG029 #4, a line that showed good induction of PURSUIT® resistance previously herein, were placed on agar supplemented with 1 mg/l of BAP and 5 μM of PURSUIT® either alone or with doxycycline 5 mg/l.

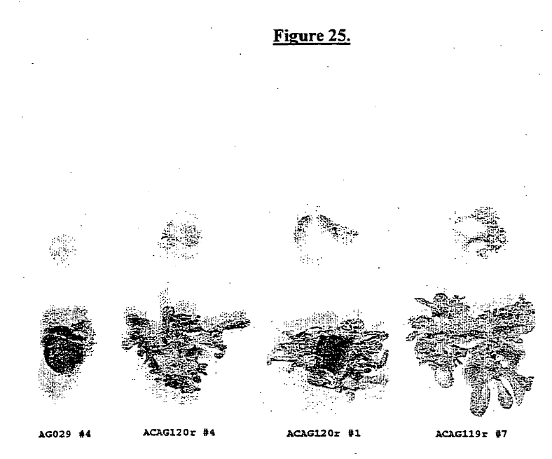

Simultaneously, another test on induction of herbicide resistance in leaf disks from the same plants floating on liquid medium was carried out. Three weeks later the phenotypic effects were evaluated. Several lines were found that responded very well to the presence of doxycycline by inducing regeneration of shoots (see FIG. 25); though uninduced tissues showed some leaky regeneration too. Line pACAG029 #4 showed very good control of herbicide resistance but the induction of regeneration was weaker. On the other hand, none of the lines transformed with pACAG119, 119r, 120, or 120r showed inducible resistance to PURSUIT® test comparable to response of pACAG029 #4 in liquid medium test.

The present invention encompasses the application of other herbicides belonging to the imazethapyr family for which the AHAS gene is known to confer resistance, which include the following, none limiting examples: imazamethabenz, imazapyr, imazaquin, etc. In addition, the invention also encompasses the application of the following, non-limiting, examples of herbicides for which the AHAS gene may also confer resistance: sulfonylurea herbicides, bensulfuron, CGA-152005, chlorimuron, chlorsulfuron, ethametsulfuron, metsulfuron, mon 12000, nicosulfuron, primisulfuron, sulfometuron, thifensulfuron, triasulfuron, tribenuron, and triflusulfuron, for example.

Arabidopsis

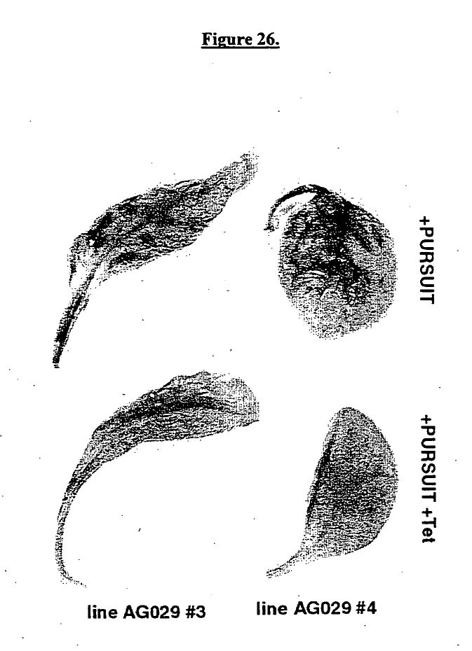

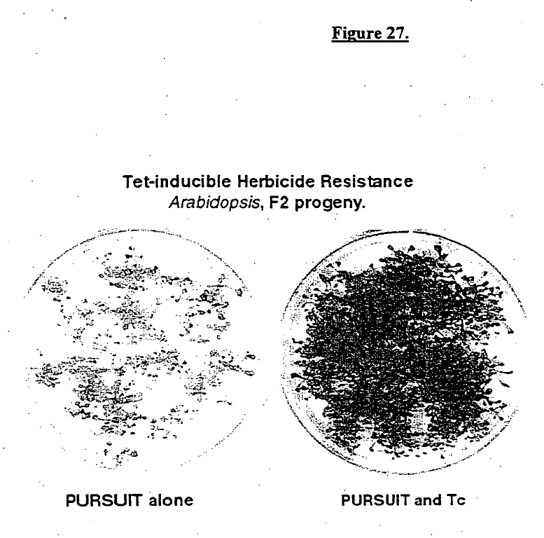

Fifteen Arabidopsis plants were selected resistant to Kanamycin after transformation with pACAG029. These lines were checked for induction of PURSUIT® resistance by cultivation of a single leaf in liquid medium containing either tet 2mg/l+PURSUIT® 1 μM or PURSUIT® 1 μM alone. Only two lines showed tet induction: green, healthy leaves were observed for leaves grown on medium containing tetracycline, compared to severely inhibited leaves grown on medium containing herbicide alone (FIG. 26). Plants were transferred to soil for seed production. Seeds from these plants were produced, collected, and planted on media with either PURSUIT® 1 μM alone or PURSUIT® 1 μM and tetracycline at 2 mg/l to evaluate the rate of herbicide resistance induction. Five lines showed induction of herbicide resistance on the medium with tetracycline. Despite the favorable results, a number of escapes were detected on the medium containing herbicide alone. The best line, AG029A # 4, which had the least number of escapes, is shown on the FIG. 27.

Based upon the results of this test, the nature of the escapes were questioned. It was proposed that the escapes are homozygous plants carrying two herbicide resistance genes. Therefore, the next step was to produce homozygous lines. For this purpose six Kanamycin-resistant plants of each of the five lines that showed induction of herbicide resistance (pACAG029 ## 1, 4, 6, 11 & 13) were transferred to soil for seed production. Two escape lines (those that grew up on PURSUIT® alone) per each line were also planted for seed production. Homozygous plants were selected for each line, however none of them were the escapes. Therefore, escape nature of some plants did not relate to homozygous state.

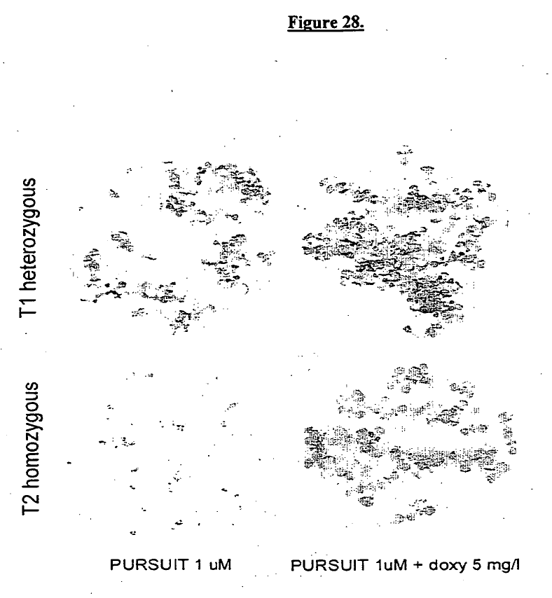

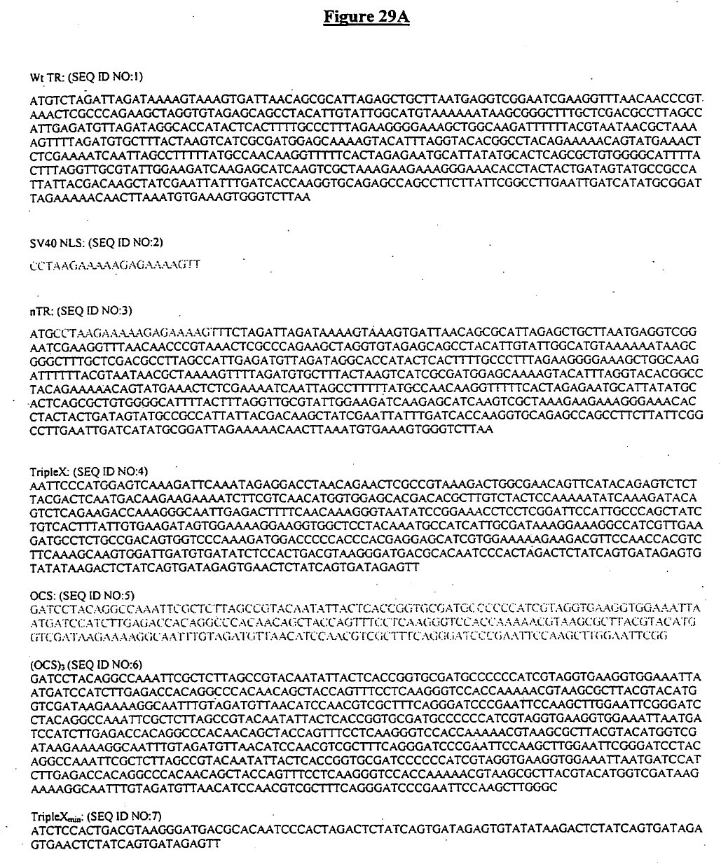

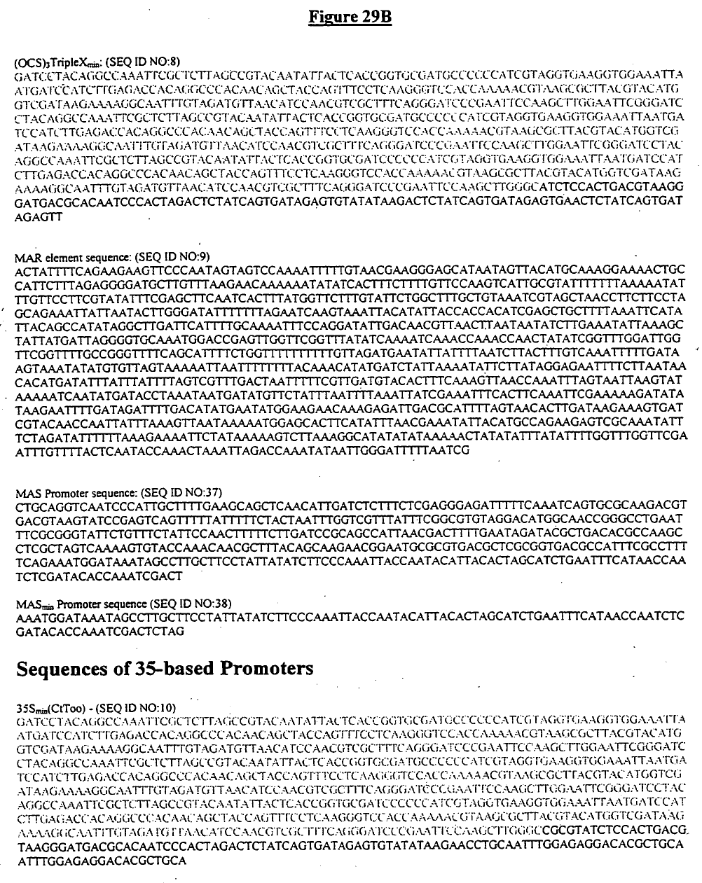

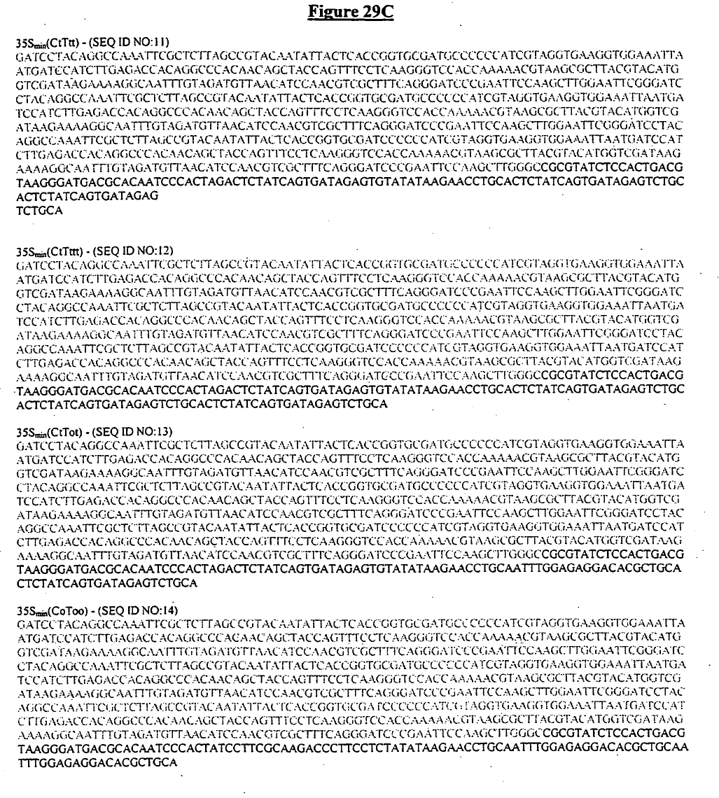

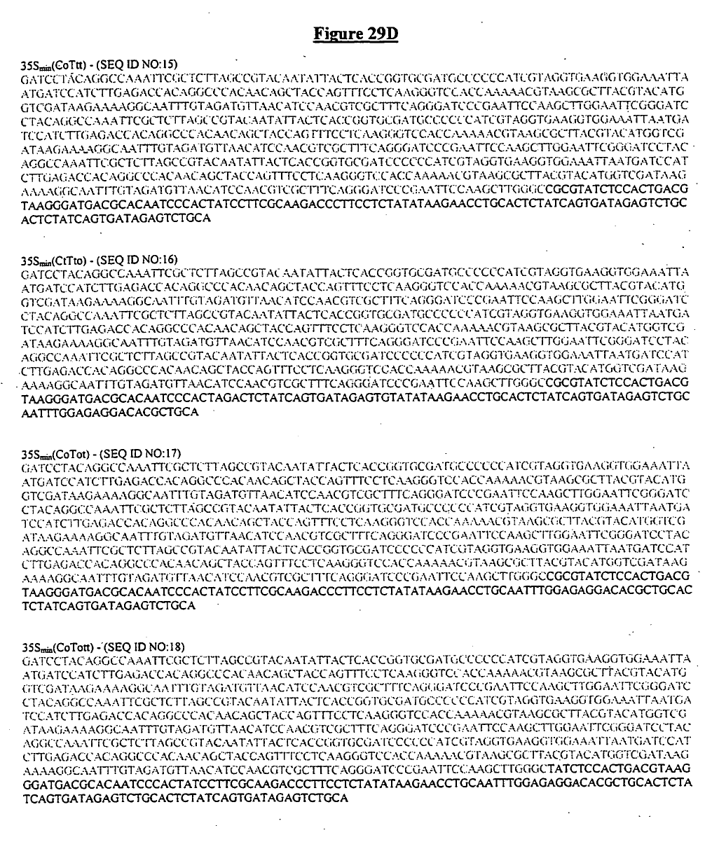

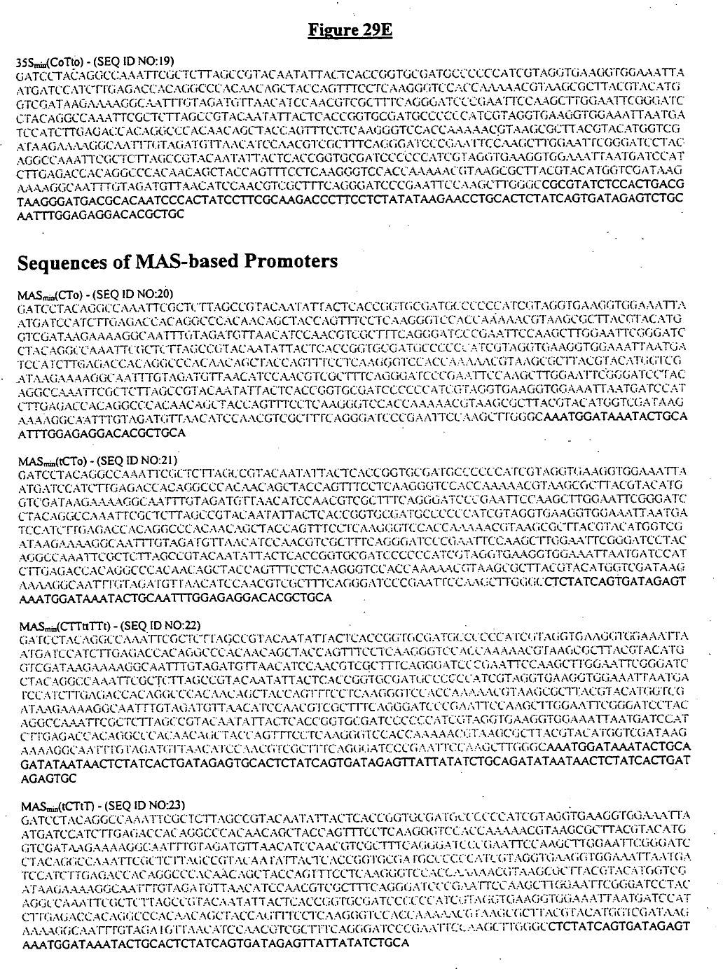

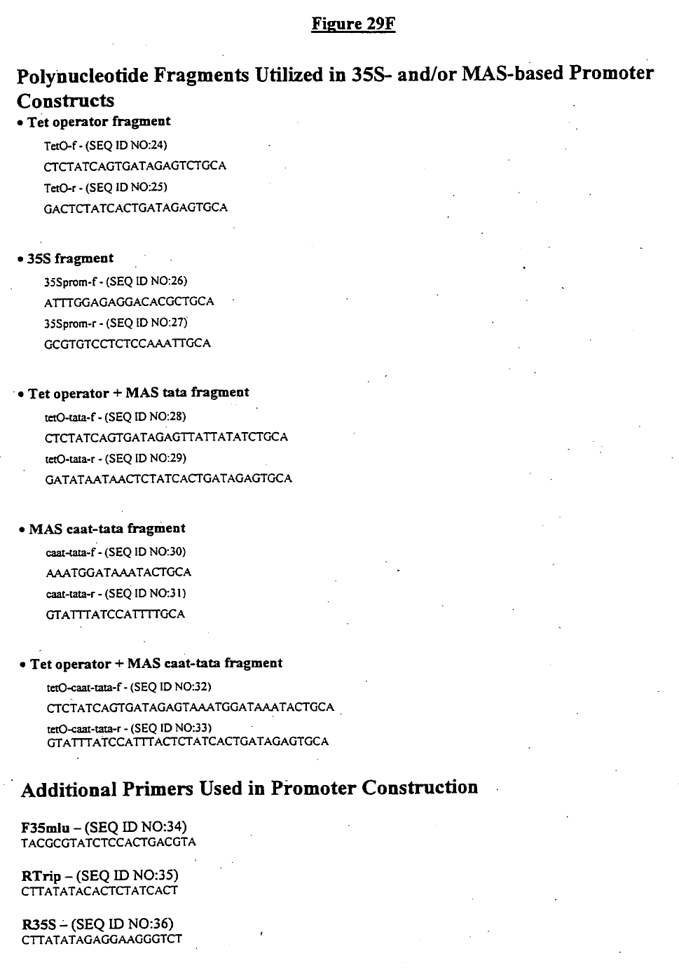

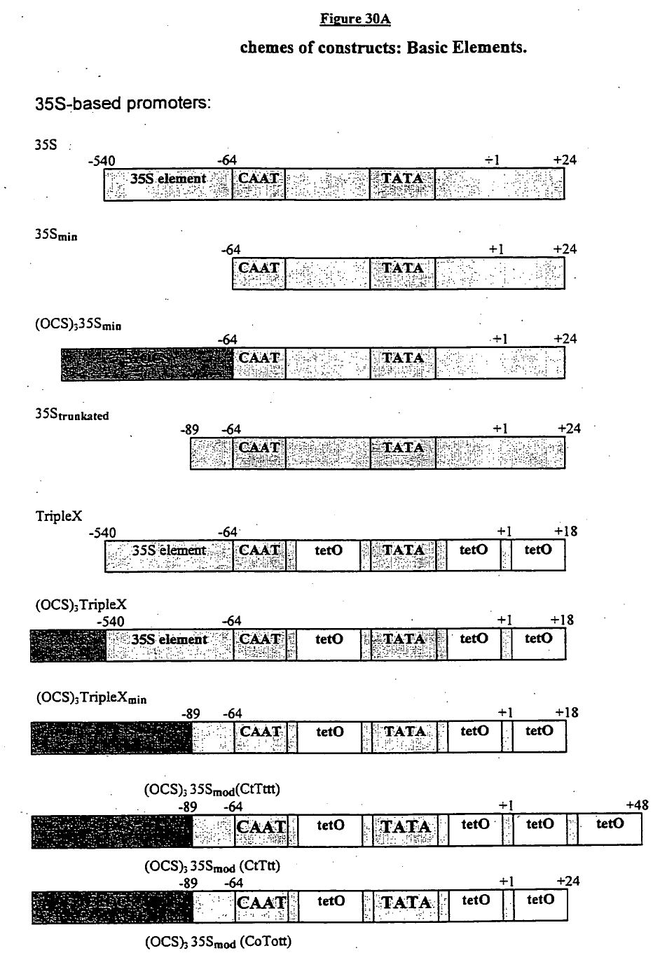

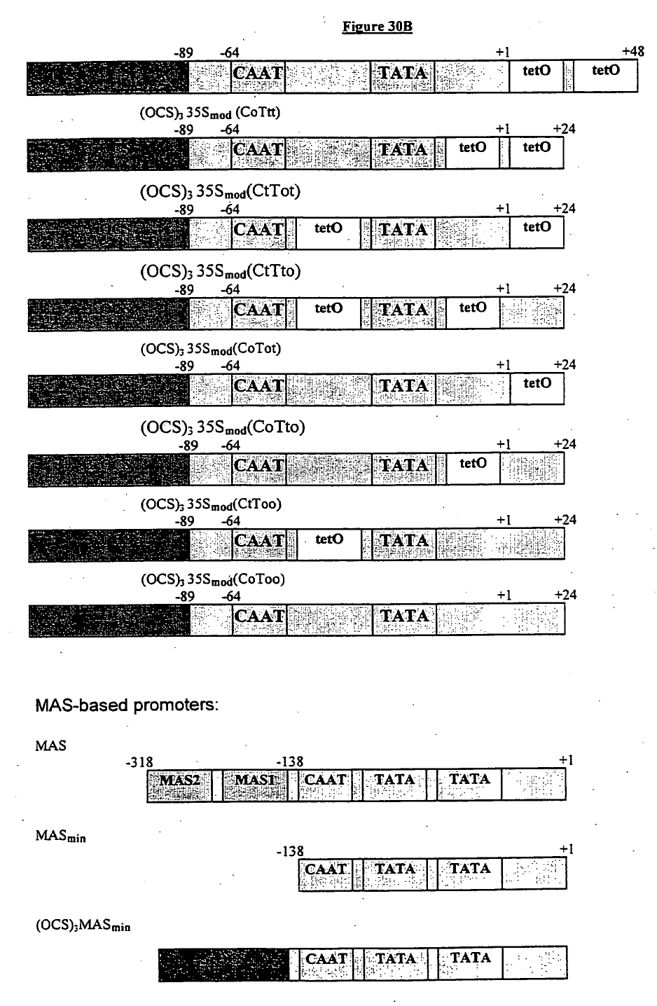

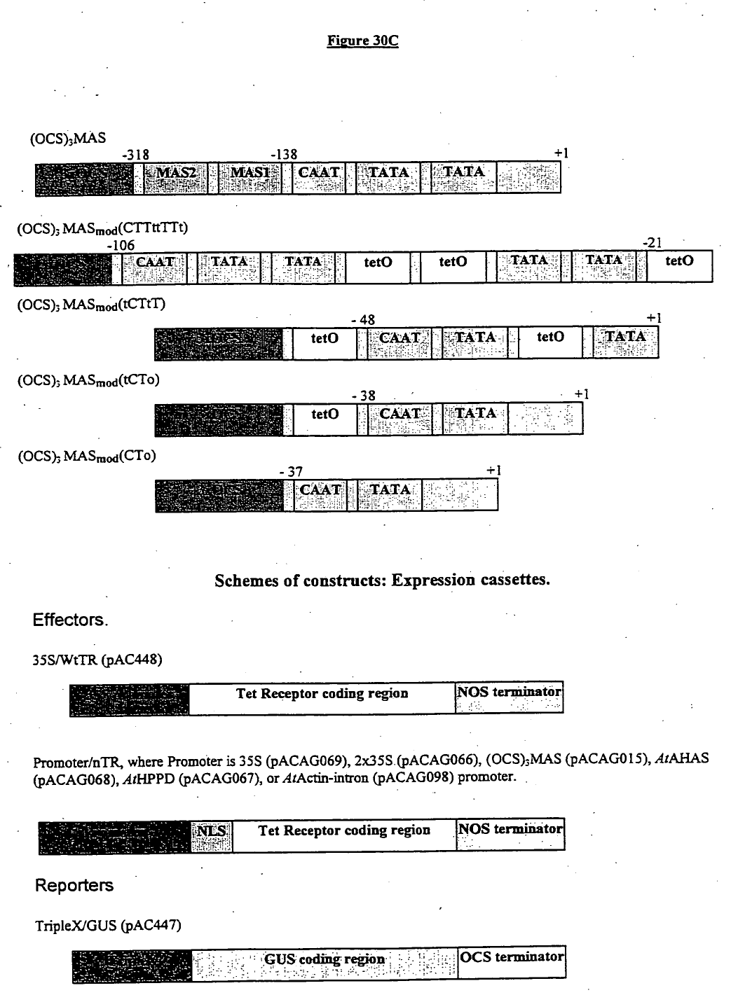

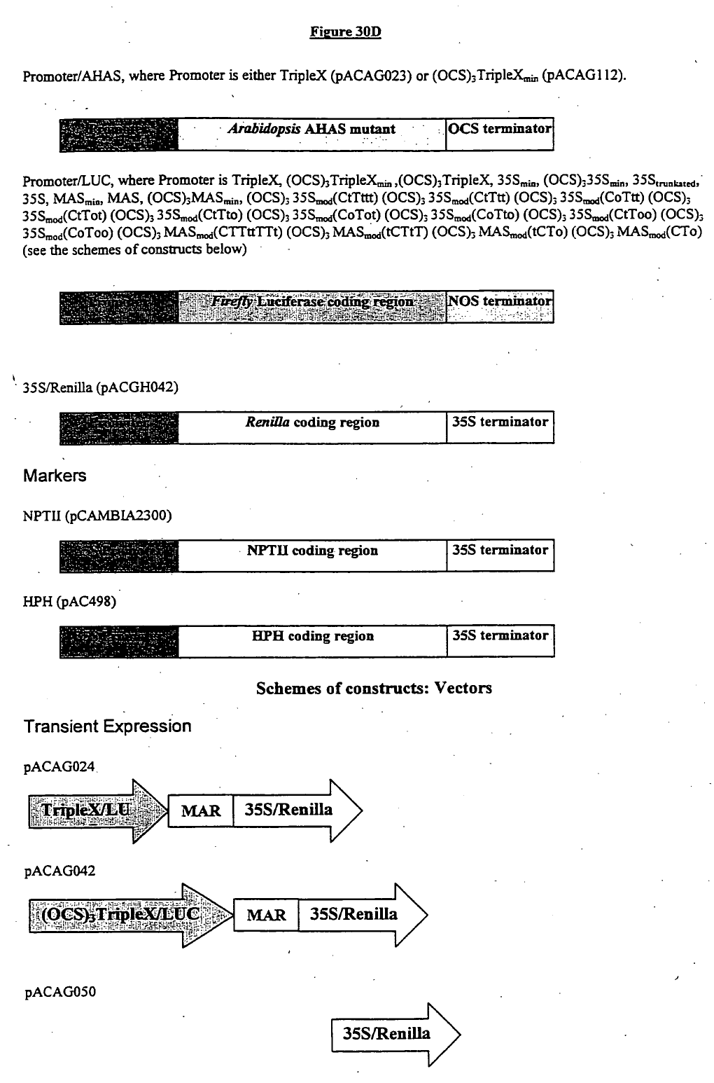

The following experiment was run in order to compare inducibility of heterozygous and homozygous lines. Five Arabidopsis lines transformed with pACAG029, T1 heterozygous and T2 homozygous seeds, were germinated on MS plates supplemented with 5 μM of PURSUIT® either alone or with doxycycline 5 mg/l. Results with one of these lines, #1, are shown in FIG. 28. As shown in FIG. 28, both T1 and T2 generations showed increased resistance to PURSUIT® in the presence of doxycycline. Moreover, herbicide resistance was repressed much stronger in homozygous lines than in heterozygous lines: the number of escapes was notably smaller in homozygous lines. Finally, the latter result was consistent among all five lines: the T2 progeny showed better repression of the inducible gene.

### Novel Tet-Inducible Promoter Cassettes Based on the 35S Promoter

In an effort to identify novel variations of the wild type 35s TripleX promoter, a set of new tet-inducible promoter cassettes was engineered on the basis of altering the number and location of the tet operators (see FIG. 32). Each of these promoter cassettes were placed upstream of the luciferase gene in an expression vector and their efficacy assessed in transient assays after co-electroporation of NT1 protoplasts with both a test vector and a plasmid carrying NLS-tet Repressor (pACAG015). Each of the co-electroporations was followed by 24-hour doxycycline (5 mg/l) induction. The results are shown in FIG. 32. Briefly, the expression from the 35s promoters showed a direct relationship to the number of tet operators on the promoter: induced expression from a promoter with only one tet operator had four fold stronger expression than a promoter with three tet operators. The same relationship was observed for background (uninduced) expression, wherein the higher the number of tet operators present within the promoter, the lower the level of background (uninduced) expression, while a decreased number of tet operators resulted in higher levels of background (uninduced) expression. Moreover, the location of tet operators on the promoter generally appeared to have little effect on the level of repression/induction. The only exception was the tet operator in the “D” position (i.e., the operator closest to the transcription start site (as in pACAG141)): promoters with a tet operator in the “D” position (for example, pACAG135, pACAG139) showed notably lower induced expression compared to promoters with the same number of operators in other locations (for example, pACAG140a). Therefore, tet inducible promoters harboring more than one tet operator (preferably two, more preferably three, and even more preferably four tet operators) are useful for decreasing the level of induced expression while concomitantly reducing the level of background expression, particularly those tet inducible promoter cassettes harboring an operator in the “D” position.

The resulting library of modified, 35S TripleX promoter-based, tet inducible promoter cassettes, are useful for a broad variety of expression applications. For example, some genes require strong repression (e.g., to avoid toxic effects on the host), yet are capable of displaying phenotypes even at low induced expression levels. An example of such a protein may be a low-copy number regulatory protein. In such instances, a promoter with three or four tet operators would be most advantageous (e.g., pACAG131, pACAG130a). On the other hand, leaky expression of other genes may generally be well tolerated in the host, but require high levels of induced expression to display a phenotype. An example of such a protein may be a high-copy number structural protein. In this instance, a promoter with one tet operator could be more advantageous (e.g., pACAG127, pACAG142a, pACAG141).

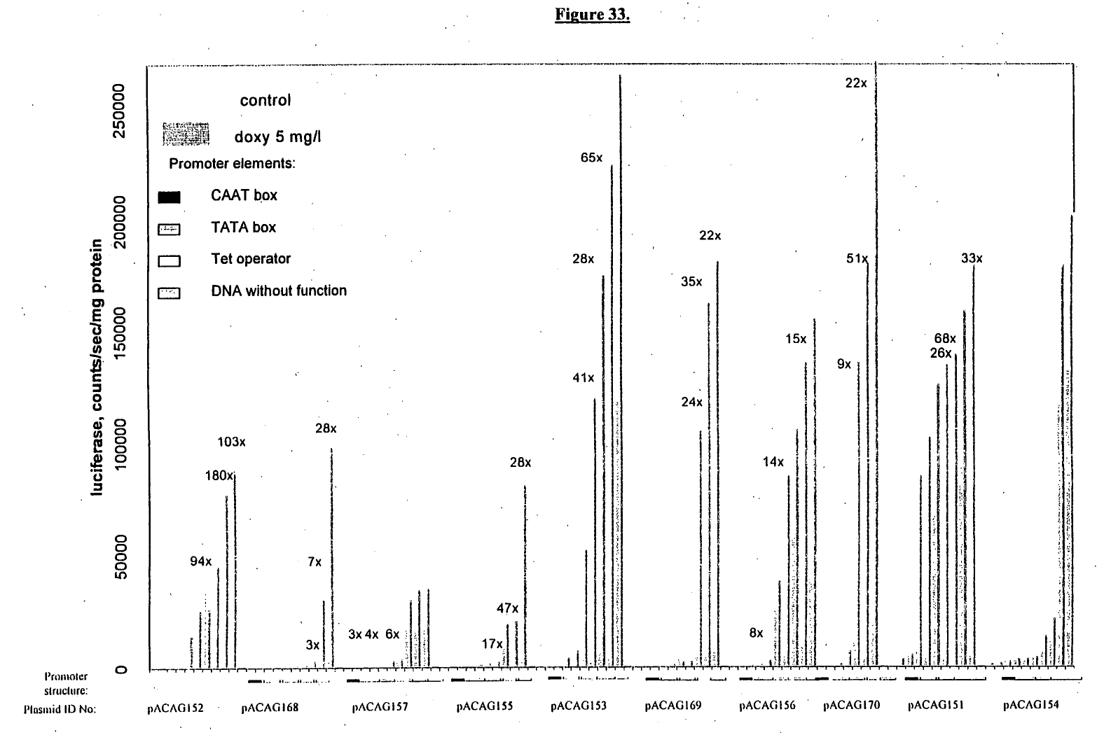

Based upon the positive results obtained in plant protoplasts, several cassettes were designed to assess the effect of each of the 35s/TripleX-based modified tet-inducible promoter cassettes on the level of Luciferase expression in plants. The cassettes were cloned into an Agrobacterium vector and were operably linked to the Luciferase coding region such that Luciferase expression was controlled by the modified tet inducible promoter cassettes of the present invention. The resulting vectors were introduced into Agrobacterium and appropriate strains were used in tobacco transformation using techniques known in the art and described elsewhere herein. The transformation produced at least 10 Kanamycin-resistant lines per cassette that were tested in a leaf disc induction assay. Ten lines per cassette were analyzed for luciferase expression. One disc from each line was incubated in liquid medium either supplemented with or without 5 mg/l of doxycycline for 5 days. Disks were collected for luciferase expression analysis. Results of the assays are presented in FIG. 33. In general, the results were in good correlation with those obtained in the transient assays described above. For example, the direct relationship between gene expression and the number of tet operators within the promoter (the more tet operators, the weaker background and, respectively, induced expression): was confirmed. However, while the transient assays emphasized the importance of having a tet operator at the “D” position within the inducible promoter cassettes of the invention (see FIG. 32), no such effect was evident in stable transformants (e.g., no difference in expression patterns was observed among promoters carrying only one tet operator, irrespective of the location of the tet operator). The latter result emphasizes the importance of transient assays in cassette optimization, as opposed to stable transformation assays in which specific effects may not be detectable.

The results of these experiments clearly demonstrate that the modified tet inducible promoter cassettes of the present invention are capable of modulating transgene expression in stable transformants. Moreover, the modified tet inducible 35S-based promoter cassettes, particularly cassettes harboring four tet operators (e.g., pACAG131), are useful for creating stable transformants in instances where the lowest possible background and high induced expression are desirable.

### Modified Tet-Inducible Promoters Based on the MAS Promoter

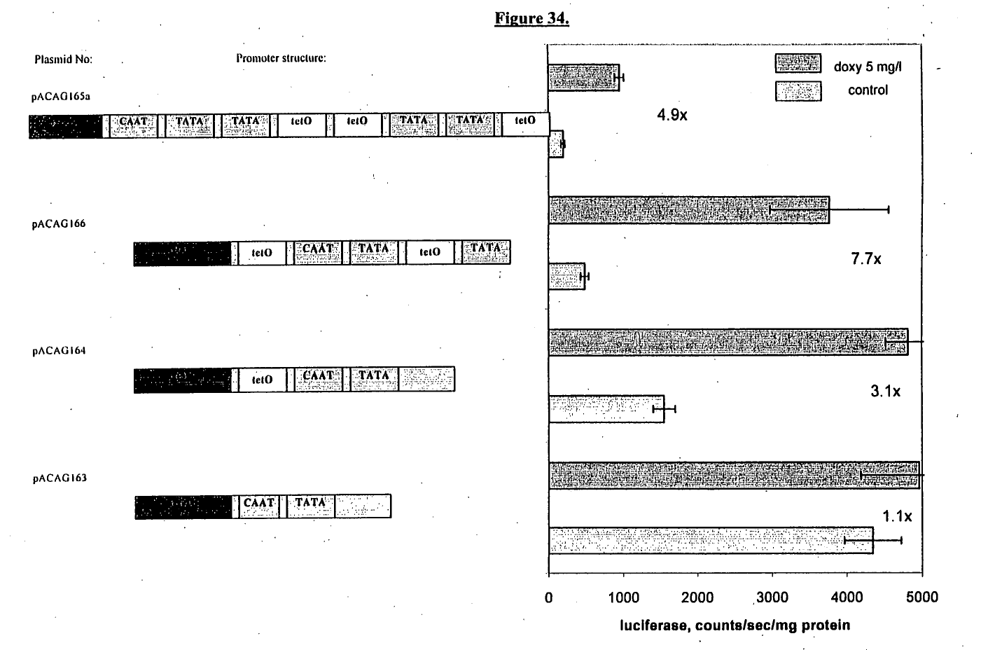

The knowledge gained through reengineering the tet-inducible 35S promoter cassettes was applied to implementing tet-induction capability into the (OCS)₃MAS promoter. Four tet-inducible (OCS)₃MAS promoters were constructed in which the number of tet operators, in addition to, their location was varied (see FIG. 34). Each of these cassettes were tested in transient assays using methods described herein. The results of the transient assays were encouraging in that each of the cassettes showed doxy-mediated induction. The results are shown in FIG. 34. In parallel to the results from the modified tet-inducible 35S-based promoters cassettes above, the number of tet operators within the MAS-based promoters cassettes appeared to be most critical in assessing the level of induced and background expression. In general, the higher the number of tet operators within the MAS-based promoters, the stronger the observed repression (i.e., background expression) and the lower the inducted gene expression. Unexpectedly, these experiments illustrated for the first time that even very short versions of the MAS promoter (as short as 37 bp containing only TATA and CAAT boxes) was capable of tet-inducible gene expression (for example, as in the case of (OCS)₃MASₘᵢₙ(CTo)).

### Orientation Effects of (OCS)₃TripleXₘᵢₙ/AHAS, Actin-Intron/nTR Cassettes

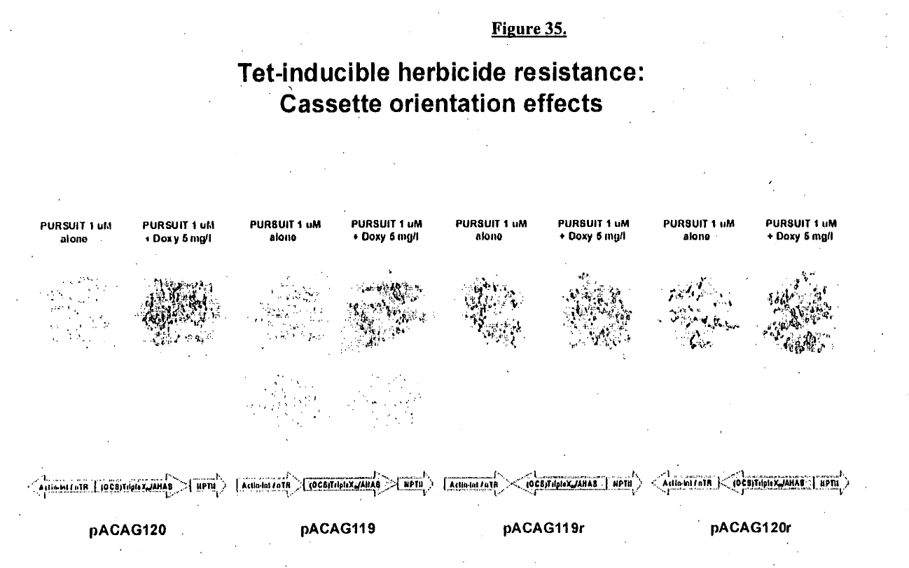

Cassette orientation effects were investigated in a set of transgenic Arabidopsis plants each transformed with the pACAG119, pACAG119r, pACAG120 or pACAG120r cassettes. Each of these cassettes comprised both an (OCS)₃TripleXₘᵢₙ/AHAS cassette and an Actin-intron/nTR cassette in varying orientations relative to each other (i.e., the orientation is a reference to the direction of transcription resulting from each promoter). The cassettes also comprised the NPTII gene coding region immediately downstream of the (OCS)₃TripleXₘᵢₙ/AHAS cassette. Seeds from each of the transformed Arabidopsis plants were germinated on suitable growth media with either PURSUIT® 1 μM alone or PURSUIT® 1 μM and doxycycline 5 mg/l. Approximately 6 to 14 transformed lines were tested for each cassette. Despite the number of lines tested for each cassette, the results were uniformly consistent for lines representing the same cassette. As a result of this uniformity, it was possible to identify doxy-induced response patterns for each of the transformed plants. The results of the assays are presented in FIG. 35.

Almost no induction was found in pACAG119r where the Actin-intron/nTR and (OCS)₃TripleXₘᵢₙ/AHAS cassettes are in opposing orientations (i.e., transcription from each of the elements is in a convergent direction). This could be explained by insufficient expression of the tet repressor due to transcriptional read-through problems. The same pattern, albeit to a lesser extent, was observed for the pACAG120r cassette: expression of the AHAS gene negatively affected the performance of the promoter driving the tet repressor cassette, Actin-intron/nTR.

In contrast, two contrasting effects were observed for the pACAG119 cassette wherein the Actin-intron/nTR and (OCS)₃TripleXₘᵢₙ/AHAS cassettes are driven in the same direction. In some of the lines, the expression of the Actin-intron/nTR was sufficient for plants to exhibit repression/derepression (upper pair), while in other lines the expression of the Actin-intron/nTR was so strong that it negatively affected AHAS expression resulting in no observable herbicide resistance (lower pair).

Unexpectedly, the optimal cassette orientation was observed for the pACAG120 cassette wherein the Actin-intron/nTR and (OCS)₃TripleXₘᵢₙ/AHAS cassettes were driven in the opposite direction (i.e., transcription from each of these cassettes was directed in divergent directions). Interestingly, significantly lower herbicide resistance was observed for pACAG029, an early generation modified tet-inducible cassette containing TripleX/AHAS and (OCS)₃S/nTR cassettes (as opposed to Actin-intron/nTR and (OCS)₃TripleXₘᵢₙ/AHAS cassettes), despite having the same relative orientation as the pACAG120 cassette. This differential herbicide resistance is believed to be solely attributable to the differential strength of each of the promoter cassettes. For example, the (OCS)₃TripleXₘᵢₙ/AHAS cassette has been shown elsewhere herein to be a stronger promoter than the TripleX/AHAS promoter. Likewise, the Actin-intron/nTR has also been shown to be a stronger promoter than the (OCS)₃MAS/nTR promoter, elsewhere herein. Preliminary experiments designed to compare the performance of T1 seeds from plants transformed with either the pACAG120 or pACAG029 cassette suggests the former performs much better than the latter with respect to tet-inducible herbicide resistance and low background expression (results not presented here).

In preferred embodiments, modified tet-inducible promoter cassettes comprising an Actin-intron/nTR cassette and a (OCS)₃TripleXₘᵢₙ/AHAS cassette in a diverging orientation, and further comprising an NPTII cassette, are useful for conferring tet inducible herbicide resistance to transformed plants and/or seeds. Also preferred are modified tet-inducible promoter cassettes comprising an Actin-intron/nTR cassette and a gene of interest under the control of the (OCS)₃TripleXₘᵢₙ promoter cassette in a diverging orientation, further comprising an NPTII cassette, which may be useful for conferring tet-inducible phenotypic traits to transformed plants, for example, as described elsewhere herein.

### Vectors and Host Cells

The present invention also relates to vectors containing the modified tetracycline inducible cassettes of the present invention, host cells, and the production of polypeptides by recombinant techniques. The vector may be, for example, a phage, plasmid, viral, or retroviral vector. Viral vectors may be replication competent or replication defective. In the latter case, viral propagation generally will occur only in complementing host cells.

The modified tetracycline inducible cassettes of the present invention may be joined to a vector containing a selectable marker for propagation in a host. Appropriate markers utilized may dependent upon the cell transfected. Generally, a plasmid vector is introduced in a precipitate, such as a calcium phosphate precipitate, or in a complex with a charged lipid. Alternatively, the plasmid vector may be transduced into the cell using PEG-mediated transfection, liposome-mediated transfection, biolistic-mediated transfection, ion beam-mediated transfection, laser-mediated transfection, in addition, to other methods known in the art. If the vector is a virus, it may be packaged in vitro using an appropriate packaging cell line and then transduced into host cells.

The invention encompasses the substitution of any of the promoters within the modified tetracycline inducible cassettes of the invention with other promoters known in the art or disclosed herein. Specifically, the following, non-limiting promoters may be substituted for any of the promoters of the present invention: the 35S promoter, MAS promoter, AtAHAS promoter, AtHPPD promoter, 45 2×35S promoter, AtActin-Intron promoter, 35S minimal promoter, MAS minimal promoter, CMV promoter, phage lambda PL promoter, the E. coli lac, trp, phoA and tac promoters, the SV40 early and late promoters and promoters of retroviral LTRs, to name a few. In addition, the promoters illustrated in Table II may also be used as a substitute for any of the promoters within modified tetracycline inducible cassettes of the invention.

In addition, it may be desirable, or necessary, in some instances to substitute the promoters within the modified tetracycline inducible cassettes of the present invention with tissue-specific or cell type-specific promoters. Examples of suitable plant-expressible promoters selectively expressed in particular tissues or cell types are well known in the art and include, but are not limited to, seed specific promoters (e.g., WO 89/03887), organ-primordia specific promoters (An et al., Plant Cell, 8:15-30, (1996)), stem-specific promoters (Keller et al., EMBO J., 7:3625-3633, (1988)), leaf specific promoters (Hudspeth et al., Plant. Mol. Biol., 12:579-589, (1989)), mesophyl-specific promoters (such as the light inducible Rubisco promoters), root-specific promoters (Keller et al., Genes Devel., 3:1639-1646, (1989)), tuber-specific promoters (Keil et al., EMBO J., 8:1323-1330, (1989)), vascular tissue specific promoters (Peleman et al., Gene, 84:359-369, (1989)), meristem specific promoters (such as the promoter of the SHOOTMERISTEMLESS (STM) gene, Long, et al., Nature, 379:66-69, (1996)), primordia specific promoter (such as the Antirrhinum CycD3a gene promoter, Doonan et al., in “Plant Cell Division” (Francis, Duditz, and Inze, Eds.), Portland Press, London, (1998)), anther specific promoters (WO 89/10396, WO 92/13956, and WO 92/13957), stigma-specific promoters (WO 91/02068), degiscence-zone specific promoters (WO 97/13865), seed-specific promoters (WO 89/03887), etc.

Additional promoters that may be substituted for a promoter within the modified tetracycline inducible cassettes of the present invention may be found in McElroy and Brettel, Tibtech, Vol. 12, February, 1994. Moreover, a number of promoters are currently being used for transformation of dicotyledonous plants. These promoters come from a variety of different sources. One group of commonly used promoters were isolated from Agrobacterium tumefaciens, where they function to drive the expression of opine synthase genes carried on the T-DNA segment that is integrated into the plant genome during infection. These promoters include the octopine synthase (OCS) promoter (L. Comai et al., 1985; C. Waldron et al., 1985), the mannopine synthase (MAS) promoter (L. Comai et al., 1985; K. E. McBride and K. R. Summerfelt, 1990) and the nopaline synthase (NOS) promoter (M. W. Bevan et al., 1983; L. Herrera-Estrella et al., 1983, R. T. Fraley et al., 1983, M. De Block et al., 1984; R. Hain et al., 1985). These promoters are active in a wide variety of plant tissue.

In addition, the promoters disclosed in the following publications may also be substituted for a promoter within the modified tetracycline inducible cassettes of the present invention: U.S. Pat. Nos. 5,623,067; 5,712,112; 5,723,751; 5,723,754; 5,723,757; 5,744,334; 5,750,385; 5,750,399; 5,767,363; 5,783,393; 5,789,214; 5,792,922; 5,792,933; 5,801,027; 5,804,694; 5,814,618; 5,824,857; 5,824,863; 5,824,865; 5,824,866; 5,824,872; and 5,929,302; and International Publication Nos. WO 97/49727, WO 98/00533, WO 98/03655, WO 98/07846, WO 98/08961, WO 98/08962, WO 98/10734, WO 98/16634, WO 98/22593, WO 98/38295, and WO 98/44097; and European Patent Application No. EP 0 846 770.

Several viral promoters are also used to, drive heterologous gene expression in dicots (J. C. Kridl and R. M. Goodman, 1986) and may be operably linked to a polynucleotide of the present invention. The Cauliflower Mosaic Virus 35S promoter is one of the promoters used most often for dicot transformation because it confers high levels of gene expression in almost all tissues (J. Odell et al., 1985; D. W. Ow et al., 1986; D. M. Shah et al., 1986). Modifications of this promoter are also used, including a configuration with two tandem 35S promoters (R. Kay et al.,1987) and the mas-35S promoter (L. Comai et al., 1990), which consists of the mannopine synthase promoter in tandem with the 35S promoter. Both of these promoters drive even higher levels of gene expression than a single copy of the 35S promoter. Other viral promoters that have been used include the Cauliflower Mosaic Virus 19S promoter (J. Paszkowski et al., 1984; E. Balazs et al.) and the 34S promoter from the figwort mosaic virus (M. Sanger et al., 1990). Other suitable promoters will be known to the skilled artisan.

TABLE II

| PROMOTER | SOURCE | LEVEL | SPECIFICITY |
|---|---|---|---|
| Monocot: | | | |
| actin | rice | high | constitutive |
| Ubiquitin | maize | high | constitutive |
| alcohol dehydrogenase | maize | ? | anaerobic stress |
| AHAS | maize | low | stem, meristem |
| (Ocs)3Mas | chimeric/synthetic | high | root preferred |
| emuchimaeric/synthetic | high | ? | |
| Dicot: | | | |
| 35S | cauliflower mosaic Virus | moderate | constitutive |
| 34S | figwort Mosaic Virus | moderate | constitutive |
| mannopine synthase | Agrobacterium T-DNA | moderate | root preferred |
| octopine synthase | Agrobacterium T-DNA | moderate | root preferred |
| nopaline synthase | Agrobacterium T-DNA | moderate | root preferred |
| actin | Arabidopsis | high | constitutive |
| Ubiquitin | potato | high | constitutive |
| Proteinase inhibitor I | tomato | high | wound-inducible, leaf |
| Proteinase inhibitor I | potato | high | wound-inducible, leaf |
| Proteinase inhibitor II | tomato | high | wound-inducible, leaf |
| Proteinase inhibitor II | potato | high | wound-inducible, leaf |
| Phaseolin | bean | high | seed |
| Ferredoxin I | pea | ? | light induced |
| AHAS | Arabidopsis | low | stem, meristem |
| HPPD | Arabidopsis | low | stem, meristem |
| Meristem-specific | Arabidopsis | | meristem |
| (Ocs)3Mas | chimeric/synthetic | high | root preferred |
| Triple X | chimeric/synthetic | moderate | tet inducible |

The expression vectors will further contain sites for transcription initiation, termination, and, in the transcribed region, a ribosome binding site for translation. The coding portion of the transcripts expressed by the vectors will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance, kanamycin resistance, hygromycin resistance, bialaphos resistance, sulfonoamide resistance, stretomycin resistance, spectinomycin resistance, chlorosulfuron resistance, glyphosphate resistance, and methotrexate resistance, for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; plant cells, and specifically plant cells and/or tissues derived from any of the plants listed in Table X. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540,, pRIT5 available from Pharmacia Biotech, Inc. Among preferred eukaryotic vectors are pWLNEO, pSV2CAT, pOG44, pXT1 and pSG available from Stratagene; and pSVK3, pBPV, PMSG and pSVL available from Pharmacia. Preferred expression vectors for use in yeast systems include, but are not limited to pYES2, pYD1, pTEF1/Zeo, pYES2/GS, PPICZ, PGAPZ, pGAPZalph, pPIC9, pPIC3.5, pHIL-D2, pHIL-S1, pPIC3.5K, pPIC9K, and PA0815 (all available from Invitrogen, Carlbad, Calif.). Preferred expression vectors in plant systems include, but are not limited to, Bin 19 (ATCC Deposit No: 37327), GA437 (ATCC Deposit No: 37350), pAK1003 (ATCC Deposit No: 37425), pAS2022 (ATCC Deposit No: 37426), pAS2023 (ATCC Deposit No: 37427), pAP2034 (ATCC Deposit No: 37428), pC22 (ATCC Deposit No: 37493), pHS24 (ATCC Deposit No: 37841), pHS85 (ATCC Deposit No: 37842), pPM1 (ATCC Deposit No: 40172), pGV3111SE (ATCC Deposit No: 53213), pCGN978 (ATCC Deposit No: 67064), pFL61 (ATCC Deposit No: 77215), pGPTV-KAN (ATCC Deposit No: 77388), pGPTV-HPT (ATCC Deposit No: 77389), pGPTV-DHFR (ATCC Deposit No: 77390), pGPTV-BAR (ATCC Deposit No: 77391), pGPTV-BLEO (ATCC Deposit No: 77392), and/or pPE1000 (ATCC Deposit No: 87573). The skilled artisan would appreciate that any of the above vectors could easily be modified to either include or delete specific elements as may be required for operability. Other suitable vectors will be readily apparent to the skilled artisan.

Introduction of the vector into the host cell can be effected by biolistic transformation, PEG-mediated transfection, calcium phosphate transfection, DEAE-dextran mediated transfection, cationic lipid-mediated transfection, electroporation, transduction, infection, or other methods known in the art or described herein. Such methods are described in many standard laboratory manuals, such as Davis et al., Basic Methods In Molecular Biology (1986). It is specifically contemplated that the modified tetracycline inducible promoter cassettes may in fact express proteins in a host cell lacking a recombinant vector (i.e., transgenic organisms).

A polypeptide expressed using a modified tetracycline inducible cassettes of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

A polypeptide expressed using a modified tetracycline inducible cassettes of the present invention can be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells.

Depending upon the host employed in a recombinant production procedure, a polypeptide expressed using a modified tetracycline inducible cassettes of the present invention may be glycosylated or may be non-glycosylated. In addition, a polypeptide expressed using a modified tetracycline promoter cassette of the present invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

The modified tetracycline inducible cassettes of the present invention may be modified to include localization signals operably linked to the modulated polynucleotide sequence of interest to provide specific cellular localization of the expressed antisense polynucleotide or polypeptide. Specifically, the modified tetracycline inducible cassettes of the present invention may include a plastid, vacuole, lysosome, peroxisome, mitochondrial, nuclear, nucleolus, microbody, endoplasmic reticulum, dictyosome, vesicle, plasma membrane, and/or golgi transit peptide or localization signal encoding sequence downstream of any promoter of the present invention and operably linked to a polynucleotide sequence or polypeptide encoding sequence.

### Uses of the Modified Tetracycline Inducible Cassettes of the Invention

The modified tetracycline inducible cassettes of the present invention have a variety of uses which include, but are not limited to, modulating the gene expression of any plant gene, either endogenous or exogenous, currently known or unknown, to that particular plant species; modulating the gene expression in a plant of any gene derived from an organism other than a plant species, currently known or unknown; modulating the expression of an antibody gene directed towards an endogenous plant protein; and/or modulating the expression of an antibody gene directed towards a pathogenic protein. In this context, as well as the contexts below, modulate should be applied to mean a quantitative, or qualitative increase, decrease, induction, or termination, of the expression levels of a gene.

Specifically, the modified tetracycline inducible cassettes of the present invention may be useful in modulating the gene expression of plant biosynthetic proteins, plant hormones, proteins involved in plant metabolism, plant defense proteins, plant salt tolerance proteins, plant water tolerance proteins, plant temperature tolerance proteins, plant structural proteins, plant nutrient uptake, external modulation of developmental timing, external modulation of environmental responses, modulating the expression of lethal genes, modulating genes that may reduce yield, modulating highly expressed genes, etc.

For example, the modified tetracycline inducible cassettes of the present invention may be useful in modulating the gene expression of proteins, which include, but are not limited to, the proteins provided in Table III.

TABLE III
Examples of Potential Coding Regions Which May be
Placed Under The Control Of Tetracycline Inducible Promoters
of the Present Invention.

```
Selectable Markers
    Antibiotic resistance
        Neomycin phosphotranseferase (NPTII)
        Hygromycin phosphotransferase (HPT)
    Herbicide tolerance
        Phosphothricine (BASTA) tolerance (PAT, BAR)
        Imidazolinone/sulfonylurea tolerance (AHAS)
        Glyphosate tolerance
Reporter genes
    b-glucuronidase (GUS)
    firefly luciferase (LUC)
    renilla luciferase (rLUC)
    Green fluorescent protein (GFP)
Agronomic traits
    Input traits
        Herbicide tolerance
            Phosphothricine (BASTA) tolerance (PAT, BAR)
            Imidazolinone/sulfonylurea tolerance (AHAS)
            Glyphosate tolerance
        Insect/fungal/viral resistance
            Bacillus thurangensis toxins (Bt)
            Pentaclethra Pentin-1 gene (WO 9854327)
        Nitrogen utilization
        Cold/drought/salt stress tolerance
        Yield enhancement
        Male sterility
        Apomixis
    Output traits
        (Alteration of quality and/or quantity)
            Lipids
            Carbohydrates
            Amino acids/proteins
            Secondary metabolites
        Production of novel metabolites
            Nutriceuticals
            Pharmaceuticals
            Cosmeceuticals
            Industrial compounds
Transcriptional activators
Functional Genomics (Genes of unknown function, Sense or
antisense orientations)
    ESTs
    CDNAs
    Genomic sequences
```

### Transgenic Methods

Another aspect of the present invention is to gene therapy methods for treating or preventing disorders, diseases and conditions. The gene therapy methods relate to the introduction of nucleic acid (DNA, RNA and antisense DNA or RNA) sequences into an organism, preferably a plant, to achieve expression of a polypeptide. This method requires a polynucleotide which codes for a polypeptide operatively linked to a promoter and any other genetic elements necessary for the expression of the polypeptide by the target tissue. Preferably a novel tetracycline operator cassette and/or novel tetracycline repressor/operator cassette is used to drive the expression of the desired polynucleotide Such transgenic and delivery techniques are known in the art, see, for example, WO90/11092, which is herein incorporated by reference.

Thus, for example, cells from a plant may be engineered with a polynucleotide (DNA or RNA) comprising a promoter, preferably a novel tetracycline operator cassette and/or novel tetracycline repressor/operator cassette of the invention, operably linked to a desired polynucleotide ex vivo, with the engineered cells then being introduced back into the plant to “treat” the deficiency. Such methods are well-known in the art and are equally applicable to plants. For example, see Belldegrun et al., J. Natl. Cancer Inst., 85:207-216 (1993); Ferrantini et al., Cancer Research, 53:107-1112 (1993); Ferrantini et al., J; Immunology 153: 4604-4615 (1994); Kaido, T., et al., Int. J. Cancer 60: 221-229 (1995); Ogura et al., Cancer Research 50: 5102-5106 (1990); Santodonato, et al., Human Gene Therapy 7:1-10 (1996); Santodonato, et al., Gene Therapy 4:1246-1255 (1997); and Zhang, et al., Cancer Gene Therapy 3: 31-38 (1996)), which are herein incorporated by reference.

As discussed in more detail below, the polynucleotide vectors can be delivered by any method that delivers injectable materials to the cells of an organism, such as, biolistic injection into the plant tissues (apical meristem, root, flower, stem, and the like). The polynucleotide vectors may be delivered in an acceptable liquid or aqueous carrier.

In one embodiment, the novel tetracycline operator cassette and/or novel tetracycline repressor/operator cassette comprising the sequence of the desired polynucleotide, is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, such vectors can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

Any strong promoter known to those skilled in the art can be used for driving the expression of the desired polynucleotide sequence within the novel tetracycline operator cassette and/or novel tetracycline repressor/operator cassette of the invention, preferably those promoters described elsewhere herein. The promoter also may be the native promoter for the desired polynucleotide.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked DNA vectors can be delivered to arteries during angioplasty by the catheter used in the procedure.

The naked polynucleotides are delivered by any method known in the art, including, but not limited to, direct needle injection at the delivery site, topical administration, and so-called “gene guns”. These delivery methods are known in the art.

The vectors may also be delivered with delivery vehicles such as viral sequences, viral particles, liposome formulations, lipofectin, precipitating agents, etc. Such methods of delivery are known in the art.

In certain embodiments, the polynucleotide vectors of the invention are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA, 84:7413-7416 (1987), which is herein incorporated by reference); mRNA (Malone et al., Proc. Natl. Acad. Sci. USA, 86:6077-6081 (1989), which is herein incorporated by reference); and purified transcription factors (Debs et al., J. Biol. Chem., 265:10189-10192 (1990), which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl Acad. Sci. USA, 84:7413-7416 (1987), which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication NO: WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., Felgner et al., Proc. Natl. Acad. Sci. USA, 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios.

Methods for making liposomes using these materials are well known in the art. For example, commercially dioleoylphosphatidyl choline (DOPC), diolecylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology, 101:512-527 (1983), which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca2+-EDTA chelation (Papahadjopoulos et al., Biochim. Biophys. Acta, 394:483 (1975); Wilson et al., Cell, 17:77 (1979)); ether injection (Deamer et al., Biochim. Biophys. Acta, 443:629 (1976); Ostro et al., Biochem. Biophys. Res. Commun., 76:836 (1977); Fraley et al., Proc. Natl. Acad. Sci. USA, 76:3348 (1979)); detergent dialysis (Enoch et al., Proc. Natl. Acad. Sci. USA, 76:145 (1979)); and reverse-phase evaporation (REV) (Fraley et al., J. Biol. Chem., 255:10431 (1980); Szoka et al., Proc. Natl. Acad. Sci. USA, 75:145 (1978); Schaefer-Ridder et al., Science, 215:166 (1982)), which are herein incorporated by reference.

Generally, the ratio of DNA to liposomes will be from about 10:1 to about 1:10. Preferably, the ration will be from about 5:1 to about 1:5. More preferably, the ration will be about 3:1 to about 1:3. Still more preferably, the ratio will be about 1:1. U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals.

In certain embodiments, cells are engineered, ex vivo or in vivo, using a retroviral particle containing RNA which comprises a sequence encoding polypeptides of the invention. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PA12, T19-14X, VT-19-17-H2, RCRE, RCRIP, GP+E-86, GP+envAm12, and DAN cell lines as described in Miller, Human Gene Therapy, 1:5-14 (1990), which is incorporated herein by reference in its entirety. The vector may transduce the packaging cells through any means known in the art. Such means include, but are not limited to, electroporation, the use of liposomes, and CaPO4 precipitation. In one alternative, the retroviral plasmid vector may be encapsulated into a liposome, or coupled to a lipid, and then administered to a host.

The producer cell line generates infectious retroviral vector particles which include novel tetracycline operator cassette and/or novel tetracycline repressor/operator cassettes of the invention comprising a desired polynucleotide. Such retroviral vector particles then may be employed, to transduce eukaryotic cells, either in vitro or in vivo. The transduced eukaryotic cells will express the desired polypeptides upon the presence of a suitable inducing agent.

In certain other embodiments, cells are engineered, ex vivo or in vivo, with novel tetracycline operator cassette and/or novel tetracycline repressor/operator cassette of the invention comprising a desired polynucleotide sequence contained in an adenovirus vector. Adenovirus can be manipulated such that it encodes and expresses polypeptides of the invention, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartzet al., Am. Rev. Respir. Dis., 109:233-238 (1974)). Finally, adenovirus mediated gene transfer has been demonstrated in a number of instances including transfer of alpha-1-antitrypsin and CFTR to the lungs of cotton rats (Rosenfeld et al., Science, 252:431-434 (1991); Rosenfeld et al., Cell, 68:143-155 (1992)). Furthermore, extensive studies to attempt to establish adenovirus as a causative agent in cancer were uniformly negative (Green et al. Proc. Natl. Acad. Sci. USA, 76:6606 (1979)).

Suitable adenoviral vectors useful in the present invention are described, for example, in Kozarsky and Wilson, Curr. Opin. Genet. Devel., 3:499-503 (1993); Rosenfeld et al., Cell, 68:143-155 (1992); Engelhardt et al., Human Genet. Ther., 4:759-769 (1993); Yang et al., Nature Genet., 7:362-369 (1994); Wilson et al., Nature, 365:691-692 (1993); and U.S. Pat. No. 5,652,224, which are herein incorporated by reference. For example, the adenovirus vector Ad2 is useful. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the preset invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, Curr. Topics in Microbiol. Immunol., 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The polynucleotide vector containing a novel tetracycline operator cassette and/or novel tetracycline repressor/operator cassette comprising a desired polynucleotide is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the novel tetracycline operator cassette and/or novel tetracycline repressor/operator cassette of the invention comprising a desired polynucleotide. These viral particles are then used to transduce eukaryotic cells, either ex vivo or in vivo. The transduced cells will contain the polynucleotide vector integrated into its genome, and will express the desired gene product in the presence of an appropriate inducer.

Another method of gene therapy involves operably associating heterologous control regions and endogenous polynucleotide sequences (e.g. encoding the polypeptide sequence of interest) via homologous recombination (see, e.g., U.S. Pat. No. 5,641,670, issued Jun. 24, 1997; International Publication NO: WO 96/29411, published Sep. 26, 1996; International Publication NO: WO 94/12650, published Aug. 4, 1994; Koller et al., Proc. Natl. Acad. Sci. USA, 86:8932-8935 (1989); and Zijlstra et al., Nature, 342:435-438 (1989). This method involves the activation of a gene which is present in the target cells, but which is not normally expressed in the cells, or is expressed at a lower level than desired. Thus, for example, a desired polynucleotide sequence could be operably inserted into a novel tetracycline operator cassette and/or novel tetracycline repressor/operator cassette of the present invention comprising flanking associating heterologous control regions and endogenous polynucleotide sequences. Such cassettes could be stably integrated into a plant cell using known techniques. In the presence of an inducing agent, the polypeptide of interest could be expressed.

Polynucleotide vectors are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence vector is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, infusion, particle accelerators, etc. The methods are described in more detail below.

The promoter-targeting sequence vector is taken up by cells. Homologous recombination between the vector and the endogenous sequence takes place, such that an endogenous sequence is placed under the control of the promoter. The promoter then drives the expression of the endogenous sequence.

Preferably, the polynucleotide encoding a desired polypeptide may contain a secretory signal sequence that facilitates secretion of the desired protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotide vectors can be used so long as the mode results in the expression of one or more molecules in an amount sufficient to provide a therapeutic effect. This includes direct needle injection, systemic injection, infusion, biolistic injectors, particle accelerators (i.e., “gene guns”), gelfoam sponge depots, other commercially available depot materials, osmotic pumps (e.g., Alza minipumps), and decanting or topical application. For example, direct injection of naked calcium phosphate-precipitated plasmid into rat liver and rat spleen or a protein-coated plasmid into the portal vein has resulted in gene expression of the foreign gene in the rat livers. (Kaneda et al., Science, 243:375 (1989)).

A preferred method of local administration is by direct injection. Preferably, a recombinant molecule of the present invention complexed with a delivery vehicle is administered by direct injection into or locally within the area of the organisms circulatory system (e.g., phloem, xylem, etc). Administration of a composition locally within the area of the organisms circulatory system refers to injecting the composition centimeters and preferably, millimeters within the organisms circulatory system.

Another method of local administration is to contact a polynucleotide vector in or around a surgical wound or grafting. For example, the polynucleotide vector can be coated on the surface of tissue inside the wound or the vector can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site.

Preferred methods of systemic administration, include injection, aerosol, percutaneous (topical) delivery. Injections can be performed using methods standard in the art. Aerosol delivery can also be performed using methods standard in the art (see, for example,. Stribling et al., Proc. Natl. Acad. Sci. USA, 189:11277-11281 (1992), which is incorporated herein by reference). Topical delivery can be performed by mixing a polynucleotide vector of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the plant or animal, the precise condition requiring treatment and its severity, and the route of administration. The frequency of treatments depends upon a number of factors, such as the amount of polynucleotide vectors administered per application, as well as the half-life of the polynucleotide and polypeptides (i.e., the effective period of application). The precise amount, number of applications and timing of applications will be determined per desired application.

Therapeutic compositions of the present invention can be administered to any organism, preferably to plants. Preferred plants include barley, oats, rye, sorghum, pea, sunflower, tobacco, cotton, petunia, tomato, broccoli, lettuce, apple, plum, orange, and lemon, and more preferably rice, maize, conola, wheat, sugerbeet, sugercane, and soybean.

Moreover, the present invention encompasses transgenic cells, including, but not limited to seeds, organisms, and plants into which genes encoding polypeptides of the present invention have been introduced. Non-limiting examples of suitable recipient plants for introducing polynucleotides of the invention, polynucleotides encoding the polypeptides of the invention, the cDNA contained in a deposit, and/or fragments, and variants therein, are listed in Table IV below.

TABLE IV
RECIPIENT PLANTS

| COMMON NAME | FAMILY | LATIN NAME |
|---|---|---|
| Maize | Gramineae | Zea mays |
| Maize, Dent | Gramineae | Zea mays dentiformis |
| Maize, Flint | Gramineae | Zea mays vulgaris |
| Maize, Pop | Gramineae | Zea mays microsperma |
| Maize, Soft | Gramineae | Zea mays amylacea |
| Maize, Sweet | Gramineae | Zea mays amyleasaccharata |
| Maize, Sweet | Gramineae | Zea mays saccharate |
| Maize, Waxy | Gramineae | Zea mays ceratina |
| Wheat, Dinkel | Pooideae | Triticum spelta |
| Wheat, Durum | Pooideae | Triticum durum |
| Wheat, English | Pooideae | Triticum turgidum |
| Wheat, Large Spelt | Pooideae | Triticum spelta |
| Wheat, Polish | Pooideae | Triticum polonium |
| Wheat, Poulard | Pooideae | Triticum turgidum |
| Wheat, Singlegrained | Pooideae | Triticum monococcum |
| Wheat, Small Spelt | Pooideae | Triticum monococcum |
| Wheat, Soft | Pooideae | Triticum aestivum |
| Rice | Gramineae | Oryza sativa |
| Rice, American Wild | Gramineae | Zizania aquatica |
| Rice, Australian | Gramineae | Oryza australiensis |
| Rice, Indian | Gramineae | Zizania aquatica |
| Rice, Red | Gramineae | Oryza glaberrima |
| Rice, Tuscarora | Gramineae | Zizania aquatica |
| Rice, West African | Gramineae | Oryza glaberrima |
| Barley | Pooideae | Hordeum vulgare |
| Barley, Abyssinian Intermediate, | Pooideae | Hordeum irregulare |
| also Irregular | | |
| Barley, Ancestral Tworow | Pooideae | Hordeum spontaneum |
| Barley. Beardless | Pooideae | Hordeum trifurcatum |
| Barley, Egyptian | Pooideae | Hordeum trifurcatum |
| Barley, fourrowed | Pooideae | Hordeum vulgare polystichon |
| Barley, sixrowed | Pooideae | Hordeum vulgare hexastichon |
| Barley, Tworowed | Pooideae | Hordeum distichon |
| Cotton, Abroma | Dicotyledoneae | Abroma augusta |
| Cotton, American Upland | Malvaceae | Gossypium hirsutum |
| Cotton, Asiatic Tree, also | Malvaceae | Gossypium arboreum |
| Indian Tree | | |
| Cotton, Brazilian, also, | Malvaceae | Gossypium barbadense |
| Kidney, and, Pernambuco | | brasiliense |
| Cotton, Levant | Malvaceae | Gossypium herbaceum |
| Cotton, Long Silk, also | Malvaceae | Gossypium barbadense |
| Long Staple, Sea Island | | |
| Cotton, Mexican, also | Malvaceae | Gossypium hirsutum |
| Short Staple | | |
| Soybean, Soya | Leguminosae | Glycine max |
| Sugar beet | Chenopodiaceae | Beta vulgaris altissima |
| Sugar cane | Woody-plant | Arenga pinnata |
| Tomato | Solanaceae | Lycopersicon esculentum |
| Tomato, Cherry | Solanaceae | Lycopersicon esculentum cerasiforme |
| Tomato, Common | Solanaceae | Lycopersicon esculentum commune |
| Tomato, Currant | Solanaceae | Lycopersicon pimpinellifolium |
| Tomato, Husk | Solanaceae | Physalis ixocarpa |
| Tomato, Hyenas | Solanaceae | Solanum incanum |
| Tomato, Pear | Solanaceae | Lycopersicon esculentum pyriforme |
| Tomato, Tree | Solanaceae | Cyphomandra betacea |
| Potato | Solanaceae | Solanum tuberosum |
| Potato, Spanish, Sweet | Convolvulaceae | Ipomoea batatas |
| potato | | |
| Rye, Common | Pooideae | Secale cereale |
| Rye, Mountain | Pooideae | Secale montanum |
| Pepper, Bell | Solanaceae | Capsicum annuum grossum |
| Pepper, Bird, also | Solanaceae | Capsicum annuum minimum |
| Cayenne, Guinea | | |
| Pepper, Bonnet | Solanaceae | Capsicum sinense |
| Pepper, Bullnose, also | Solanaceae | Capsicum annuum grossum |
| Sweet | | |
| Pepper, Cherry | Solanaceae | Capsicum annuum cerasiforme |
| Pepper, Cluster, also Red | Solanaceae | Capsicum annuum fasciculatum |
| Cluster | | |
| Pepper, Cone | Solanaceae | Capsicum annuum conoides |
| Pepper, Goat, also Spur | Solanaceae | Capsicum frutescens |
| Pepper, Long | Solanaceae | Capsicum frutescens longum |
| Pepper, Oranamental Red, | Solanaceae | Capsicum annuum abbreviatum |
| also Wrinkled | | |
| Pepper, Tabasco Red | Solanaceae | Capsicum annuum conoides |
| Lettuce, Garden | Compositae | Lactuca sativa |
| Lettuce, Asparagus, also | Compositae | Lactuca sativa asparagina |
| Celery | | |
| Lettuce, Blue | Compositae | Lactuca perennis |
| Lettuce, Blue, also Chicory | Compositae | Lactuca pulchella |
| Lettuce, Cabbage, also | Compositae | Lactuca sativa capitata |
| Head | | |
| Lettuce, Cos, also Long- | Compositae | Lactuca sativa longifolia |
| leaf, Romaine | | |
| Lettuce, Crinkle, also | Compositae | Lactuca sativa crispa |
| Curled, Cutting, Leaf | | |
| Celery | Umbelliferae | Apium graveolens dulce |
| Celery, Blanching, also | Umbelliferae | Apium graveolens dulce |
| Garden | | |
| Celery, Root, also Turniprooted | Umbelliferae | Apium graveolens rapaceum |
| Eggplant, Garden | Solanaceae | Solanum melongena |
| Sorghum | Sorghum | All crop species |
| Alfalfa | Leguminosae | Medicago sativum |
| Carrot | Umbelliferae | Daucus carota sativa |
| Bean, Climbing | Leguminosae | Phaseolus vulgaris vulgaris |
| Bean, Sprouts | Leguminosae | Phaseolus aureus |
| Bean, Brazilian Broad | Leguminosae | Canavalia ensiformis |
| Bean, Broad | Leguminosae | Vicia faba |
| Bean, Common, also | Leguminosae | Phaseolus vulgaris |
| French, White, Kidney | | |
| Bean, Egyptian | Leguminosae | Dolichos lablab |
| Bean, Long, also Yard- | Leguminosae | Vigna sesquipedalis |
| long | | |
| Bean, Winged | Leguminosae | Psophocarpus tetragonolobus |
| Oat, also Common, Side, | Avena | Sativa |
| Tree | | |
| Oat, Black, also Bristle, | Avena | Strigosa |
| Lopsided | | |
| Oat, Bristle | Avena | |
| Pea, also Garden, Green, | Leguminosae | Pisum, sativum sativum |
| Shelling | | |
| Pea, Blackeyed | Leguminosae | Vigna sinensis |
| Pea, Edible Podded | Leguminosae | Pisum sativum axiphium |
| Pea, Grey | Leguminosae | Pisum sativum speciosum |
| Pea, Winged | Leguminosae | tetragonolobus purpureus |
| Pea, Wrinkled | Leguminosae | Pisum sativum medullare |
| Sunflower | Compositae | Helianthus annuus |
| Squash, Autumn, Winter | Dicotyledoneae | Cucurbita maxima |
| Squash, Bush, also Summer | Dicotyledoneae | Cucurbita pepo melopepo |
| Squash, Turban | Dicotyledoneae | Cucurbita maxima turbaniformis |
| Cucumber | Dicotyledoneae | Cucumis sativus |
| Cucumber, African, also | | Momordica charantia |
| Bitter | | |
| Cucumber, Squirting, also | | Ecballium elaterium |
| Wild | | |
| Cucumber, Wild | | Cucumis anguria |
| Poplar, California | Woody-Plant | Populus trichocarpa |
| Poplar, European Black | | Populus nigra |
| Poplar, Gray | | Populus canescens |
| Poplar, Lombardy | | Populus italica |
| Poplar, Silverleaf, also | | Populus alba |
| White | | |
| Poplar, Western Balsam | | Populus trichocarpa |
| Tobacco | Solanaceae | Nicotiana |
| Arabidopsis Thaliana | Cruciferae | Arabidopsis thaliana |
| Turfgrass | Lolium | |
| Turfgrass | Agrostis | |
| | Other families of turfgrass | |
| Clover | Leguminosae | |

### Plant Hormones

The modified tetracycline inducible cassettes of the present invention may be useful in modulating the gene expression of the following, non-limiting, plant hormone proteins: auxins, indoleacetic acid, gibberellins, cytokinins, ethylene, abscisic acid, polyamines, jasmonates, tuberonic acid, salicylic acid, systemin, brassinolides, zeatin; and specifically, indole-3-acetic acid, indole-3-butyric acid, 4-chloroindole-3-acetic acid, indole-3-acetyl-1-O-B-D-glucose, indole-3-acetyl-myo-inositol, jasmonic acid, methyl jasmonate, kinetin, including any known derivatives of the hormones described above, etc. Additional examples of plant hormones are known in the art (see, for example, Davies, P. J., in “Plant Hormones: Physiology, Biochemistry, and Molecular Biology”, Kluwer Academic Publishers, Boston, 1995; which is hereby incorporated by reference in its entirety herein).

In the process of modulating plant auxin levels, the modified tetracycline inducible cassettes of the present invention may necessarily be capable of the following, non-limiting, effects on a plant: stimulating cell enlargement, stimulating stem growth, stimulating cell division in the cambium, stimulating differentiation of phloem and xylem, stimulating root initiation on stem cuttings, stimulating the development of branch roots, stimulating the differentiation of roots, mediating the bending (tropistic) response of shoots and roots to gravity and light, repression of lateral buds, delay of leaf senescence, inhibition or promotion of leaf and fruit abscission (via ethylene), induction of fruit setting and growth, enhancement of assimilate transport via phloem, delay of fruit ripening, promotion of flowering in Bromeliads, stimulating flower growth, promotion of femaleness in dioecious flowers, and stimulating the production of ethylene, for example.

In the process of modulating plant gibberellin levels, the modified tetracycline inducible cassettes of the present invention may necessarily be capable of the following, non-limiting, effects on a plant: stimulating cell division and cell elongation, inducing hyperelongation, inducing bolting, inducing stem elongation in response to long days, inducing germination, inducing germination in seeds in the absence of stratification or hardening, stimulating production of a-amylase, inducing fruit setting and growth, and inducing maleness in dioecious flowers, for example.

In the process of modulating plant cytokinin levels, the modified tetracycline inducible cassettes of the present invention may necessarily be capable of the following, non-limiting, effects on a plant: inducing cell division in the presence of auxin, inducing cell division in crown gall tumors, inducing cell division in apical meristem, inducing cell division in rapidly dividing cells, promoting shoot initiation, inducing bud formation, inducing growth of lateral buds, releasing lateral bud growth from apical dominance, inducing cell enlargement, inducing leaf expansion, enhancing stomatal opening, stimulating the accumulation of chlorophyll, inducing the conversion of etioplasts to chloroplasts, and delaying leaf senescence, for example.

In the process of modulating plant ethylene levels, the modified tetracycline inducible cassettes of the present invention may necessarily be capable of the following, non-limiting, effects on a plant: releasing the plant from dormancy, inducing shoot and root growth and differentiation, inducing adventitious root formation, inducing leaf and fruit abscission, inducing flowering, inducing femaleness in dioecious flowers, inducing flower opening, inducing flower and leaf senescence, and inducing fruit ripening, for example.

In the process of modulating plant abscisic acid levels, the modified tetracycline inducible cassettes of the present invention may necessarily be capable of the following, non-limiting, effects on a plant: inducing stomatal closure, inhibition of shoot growth, inducing storage protein synthesis in seeds, inhibition of a-amylase production in germinating cereal grains, induction of some aspects of dormancy, and induction of proteinase inhibitor synthesis, for example.

In the process of modulating plant polamine levels, the modified tetracycline inducible cassettes of the present invention may necessarily be capable of the following, non-limiting, effects on a plant: regulation of growth and development of plant cells and tissues, modulating the synthesis of macromolecules, modulating the activity of macromolecules, stabilizing cellular plasma membrane, decreasing leakage of betacyanin from wounded tissue, preservation of thylakoid structure in excised barley leaves, counteraction of hormone-induced affects on the cell membrane, binding to nucleic acids, protection of nucleic acids from alkylating agents, controlling chromosome condensation, controlling nuclear membrane dissolution during pre-prophase, and modulating the structure and function of tRNA's, for example.

In the process of modulating plant jasmonate levels, the modified tetracycline inducible cassettes of the present invention may necessarily be capable of the following, non-limiting, effects on a plant: inhibition of plant growth, inhibition of seed germination, promotion of senescence, promotion of abscission, promotion of tuber formation, promotion of fruit ripening, promotion of pigment formation, promotion of tendril coiling, induction of proteinase inhibitors, and inhibit insect infestation, for example.

In the process of modulating.plant salicylic acid levels, the modified tetracycline inducible cassettes of the present invention may necessarily be capable of the following, non-limiting, effects on a plant: induction of thermogenesis, providing resistance to pathogens via induction of pathogenesis related proteins, enhancement of flower longevity, inhibition of ethylene biosynthesis, inhibition of seed germination, inhibiting the wound response, counteracting the plants response to abscisic acid, for example.

In the process of modulating plant brassinosteroid levels, the modified tetracycline inducible cassettes of the present invention may necessarily be capable of the following, non-limiting, effects on a plant: promotion of stem elongation, inhibition of root growth, inhibition of root development, promotion of ethylene biosynthesis, and promotion of epinasty, for example.

The modified tetracycline inducible cassettes of the present invention may modulate the expression of one, two, three, or more, or any combination of the above, hormones in a plant. Additional effects of hormones on a plant, including its cells, tissues, and organs are known in the art and the aforementioned plant hormone effects should not be construed as limiting the utility of any of the modified tetracycline inducible cassettes of the present invention.

### Plant Defense

The modified tetracycline inducible cassettes of the present invention may be useful in modulating the gene expression of genes capable of increasing a plants defense mechanisms against either environmental or pathogenic stresses (e.g., viral, fungal, mycoplasma, bacterial, nematode, herbicidal, insecticidal, acid rain, drought, chemical, etc.). Such defense mechanisms may be a combination of structural characteristics (i.e., to serve as a physical barrier to inhibit a pathogen, for example, from entering or spreading throughout the plant), and biochemical reactions either on the scale of the whole plant or of individual cells (e.g., producing substances that are either toxic to the pathogen, or create an environment that is non-permissive for pathogen survival, etc.).

Structurally, the modified tetracycline inducible cassettes of the present invention may be useful in modulating the gene expression of genes useful for increasing the number of trichomes; increasing the thickness and/or composition of wax secretions or the waxy layer, increasing the thickness and/or composition of the cuticle, altering the structure of the epidermal cell wall, altering the size, shape, and/or location of the stomata and lenticels, inducing the plant to create or increase a layer of thick-walled cells (e.g., cork cell layer, etc.), increasing the thickness and/or composition of the outer epidermal cell wall, inducing the formation of an abscission layer, induce the formation of tyloses, induce the production and/or deposition of gums, inducing the thickening of the outer parenchyma cell layer of the cell wall, inducing the thickening of the cell wall, inducing the deposition of callose papillae in the inner layer of the cell wall, inducing a necrotic or hypersensitive defense reaction in cells and/or tissues (i.e., cell death), inducing the polymerization of oxidized phenolic compounds into lignin-like substances to structurally interfere with pathogen development, and/or inducing a cytoplasmic defense reaction.

Biochemically, the modified tetracycline inducible cassettes of the present invention may be useful in modulating the gene expression of genes useful for releasing pathogenic inhibitors into the plants environment, releasing fungitoxic exudates, and/or releasing phenolic compounds (e.g., protocatechioc acid, catechol, etc.). Alternatively, the modified tetracycline inducible cassettes of the present invention may be useful in modulating the gene expression of genes useful for increasing the synthesis of phenolic compounds (e.g., chlorogenic acids, caffeic acids, scopoletin, oxidation products of phenolic compounds, phytoalexins (see, Bell, et al., Ann. Rev. Plant Physiol, 32, 1981, for specific examples of phytoalexins), phaseolin, rishitin, kievitone, pisatin, glyceollin, gossypol, capsidiol, etc.), tannins, and/or saponins (e.g., tomatine, avenacin, etc.) within the cells and tissues of the plant. Alternatively, the modified tetracycline inducible cassettes of the present invention may be useful in modulating the gene expression of genes useful for increasing the expression of plant hydrolytic enzymes (e.g., glucanases, chitinases, etc.) that may cause degradation of the pathogen cell wall, etc.

In another embodiment, the modified tetracycline inducible cassettes of the present invention may be useful in modulating the gene expression of genes useful for inhibiting the expression of recognition factors essential for host-pathogen interaction (e.g., specific oligosaccarides, carbohydrate moieties, receptors, ligands, proteins, glycoproteins, lectins, etc.). For example, the modified tetracycline inducible cassettes of the present invention may be useful in modulating the gene expression of genes useful for inhibiting the expression of a protein that serves as a target for a pathogenic toxin, thus rendering the host in-sensitive to the toxin.

In another embodiment, the modified tetracycline inducible cassettes of the present invention may be useful in modulating the gene expression of genes useful in inhibiting the ability of the plants metabolic machinery to complete essential steps required for a competent pathogenic response (e.g., inhibiting the ability of plant ribosomes to recognize the pathogens nucleic acid, such as a viral nucleic acid; and/or inhibiting the ability of the plants DNA polymerase machinery to recognize and/or synthesize pathogenic DNA; or inhibiting the plants ability to catalyze a specific enzymatic step essential to eliciting a pathogenic response, etc.)

In yet another embodiment, the modified tetracycline inducible cassettes of the present invention may be useful in modulating the gene expression of genes useful for inhibiting either the production or transport or retention of essential nutrients required for a permissive pathogenic infection (e.g., inhibiting the transport of non-essential minerals or vitamins required for a pathogenic response, etc.).

In one embodiment of the invention, the modified tetracycline inducible cassettes of the present invention may be useful in modulating the gene expression of genes useful for increasing the expression or activity of phenol oxidizing enzymes (e.g., polyphenoloxidases, peroxidase, etc.), increasing the expression or activity of phenylalanine ammonia lyase, increasing the activity or expression of proteins capable of forming pectin salts or pectin complexes, etc.

In a further embodiment, the modified tetracycline inducible cassettes of the present invention may be useful in modulating the gene expression of genes useful for either directly or indirectly inhibiting the activity of a pathogenic protein essential to eliciting an infection (e.g., inhibiting the enzymatic activity of the protein, such as for a hydrolytic enzyme, for example, inhibiting the proteins ability to bind to a receptor or ligand, inhibiting protein-protein or protein-DNA interactions of the pathogenic protein, etc.). Specifically, the modified tetracycline inducible cassettes of the present invention may be useful in modulating the gene expression of genes useful for either directly or indirectly inhibiting wildfire toxin, chlorosis-inducing toxins, tabtoxin, phaseoloyoxin, rhizobitoxine, wilt-inducing bacterial polysaccarides, amylovorin, glycopeptide toxins, peptide toxins, syringomycin, tagetitoxin, helminthosporoside, victorin, helminthospoium maydis T-toxin, helminthospoium carbonum toxin, periconia circinata toxin, phyllosticta maydis toxin, alternaria toxins, fusarial wilt toxins, ophiobolin, helminthosporal, terpinoid toxins, fusicoccin, pyricularin, colletotin, alternaric acid, tentoxin, phytotoxins, zinniol, tentoxin, ascochitine, diaporthin, skyrin, Didymella applanata toxin, Myrothecium roridum toxin, Leptosphaerulina briosiana toxin, Alternaria tenuis phenolic toxins, Cercospora beticola toxin, Verticillium albo-atrum toxin, Phytophthora nicotianae var. parasitica toxin, Phytophthora megasperma var. sojae toxin, Ceratocystis ulmi toxins, peptidorhamnomannan, Stemphylium botryosum toxins, stemphylin, stemphyloxin, Pyrenophora teres toxins, N-(2-amino-2-carboxyethyl) aspartic acid, aspergillomarasmine A, and Rhynchosporosides toxins, for example.

In another embodiment, the modified tetracycline inducible cassettes of the present invention may be useful in modulating the gene expression of genes useful for either increasing or inducing the production of cyanogenic glycosides or esters, increasing the activity or expression of hydrolytic enzymes capable of hydrolyzing cyanogenic glycosides or esters, increasing the activity or expression of enzymes capable of releasing cyanide into plant cells and tissues, increasing the activity or expression of enzymes capable of detoxifying cyanide (e.g., formamide hydro-lyase, etc.) and/or increasing the expression of b-proteins, etc.

In another embodiment, the modified tetracycline inducible cassettes of the present invention may be useful in modulating the gene expression of genes useful for either increasing or inducing the production of secondary metabolites, which include, but are not limited to the following: acetyl salicylic acid, aconitine, atropine, cytisine, germinine, cardiac glycosides (e.g., calotropin, oleandrin, etc.), linarine, quinine, atropine, taxine, cicutoxin, hyoscyamine, pyrethrin, rotenone, camphor, etc.

In another embodiment, the modified tetracycline inducible cassettes of the present invention may be useful in modulating the gene expression of genes useful for either increasing or inducing the production of non-protein amino acids, which include, but are not limited to the following: b-cyanoalanine, azetidine 2-carboxylic acid, canavanine, 3,4-dihyroxyphenylalanine, etc.

In another embodiment, the modified tetracycline inducible cassettes of the present invention may be useful in modulating the gene expression of genes useful for either increasing or inducing the production of terpenes, which include, but are not limited to the following: 1.8 cineole, camphor, a-pinene, b-pinene, camphene, thujone, etc.

Alternatively, the modified tetracycline inducible cassettes of the present invention may be useful in modulating the gene expression of genes useful for directly or indirectly inhibiting an infectious agent, without necessarily increasing the plants defense mechanisms.

The present invention encompasses the application of one, two, three, four, or more, including any combination thereof, of any of the methods of increasing plant defense mechanisms against either an environmental or infectious agent described above and elsewhere herein. Additional methods of increasing a plants defense mechanisms are known in the art. Additionally, a list of compounds and/or proteins that could serve as targets for increased production or expression by the use of a polynucleotide or polypeptide of the present invention to increase a plants defense mechanisms are known in the art (see, for example, Agrious, N. C., supra; Goodman, R. N., in “The Biochemistry and Physiology of Plant Disease”, University of Missouri Press, Columbia, 1986; and Lambers, H., et al., in “Plant Physiological Ecology”, Spinger-Verlag, New York, (1998); which are hereby incorporated herein by reference in their entirety).

### Nutrients

The modified tetracycline inducible cassettes of the present invention may be useful in modulating the gene expression of genes capable of modulating the plants nutritional status. For example, the modified tetracycline inducible cassettes of the resent invention may be useful in modulating the gene expression of genes capable of modulating the plants ability to retain a particular nutrient, to modulate the plants ability to synthesize a particular nutrient, to modulate the plants ability to assimilate a nutrient, to modulate the plants ability to absorb or uptake a particular nutrient, to modulate the plants ability to transport a particular nutrient, to modulate the plants ability to store a particular nutrient, to modulate the plants ability to survive under nutrient deficiencies, and to prevent, detect, and/or provide resistance to nutrient deficiency symptoms and traits.

Specific examples of nutrients that may be modulated in a plant using the modified tetracycline inducible cassettes of the present invention include the following, non-limiting, nutrients: carbon, hydrogen, oxygen, nitrogen, phosphorus, sulfur, potassium, calcium, magnesium, boron, chlorine, copper, iron, manganese, zinc, molybdenum, cobalt, selenium, silicon, sodium, nickel, water, carbon dioxide, in addition to metabolic by-products, etc. Additional nutrients essential to maintaining plant homeostasis are known in the art.

In the process of modulating plant boron levels, the modified tetracycline inducible cassettes of the present invention may be useful in preventing, detecting, alleviating, and/or conferring resistance to the following, non-limiting, symptoms of plant boron deficiency: terminal leaf necrosis, premature leaf abscission layer formation, terminal shoot internode shortening, blackening and/or death of apical meristem tissue, shortening of root shoots, plant dwarfing, plant stunting, impairment of flower development, impairment of seed development, etc.

In the process of modulating plant calcium levels, the modified tetracycline inducible cassettes of the present invention may be useful in preventing, detecting, alleviating, and/or conferring resistance to the following, non-limiting, symptoms of plant calcium deficiency: chlorotic leaves, leaf curling, leaf rolling, degradation of meristematic tissues in stems and roots, meristematic tissue death, decreased root development, decreased root fiber content, decreased fruit development, etc.

In the process of modulating plant chlorine levels, the modified tetracycline inducible cassettes of the present invention may be useful in preventing, detecting, alleviating, and/or conferring resistance to the following, non-limiting, symptoms of plant chlorine deficiency: leaf tip wilting, leaf chlorosis, leaf bronzing, basipetal leaf necrosis proximal to areas of wilting, etc.

In the process of modulating plant copper levels, the modified tetracycline inducible cassettes of the present invention may be useful in preventing, detecting, alleviating, and/or conferring resistance to the following, non-limiting, symptoms of plant copper deficiency: terminal shoot wilting, terminal shoot death, fading of leaf color, reduction of carotene in plant cells and tissues, reduction of other pigments in plant cells and tissues, etc.

In the process of modulating plant iron levels, the modified tetracycline inducible cassettes of the present invention may be useful in preventing, detecting, alleviating, and/or conferring resistance to the following, non-limiting, symptoms of iron deficiency: interveinal white chlorosis of young leaves first, chlorisis of aerial tissues, aerial tissue necrosis, bleaching of leaves, scorching of leave margins and tips, etc.

In the process of modulating plant magnesium levels, the modified tetracycline inducible cassettes of the present invention may be useful in preventing, detecting, alleviating, and/or conferring resistance to the following, non-limiting, symptoms of magnesium deficiency: mottling chlorosis with green veins and leaf web tissue yellow or white on old leaves first, wilting of leaves, formation of leaf abscission layer in the absence of the wilting stage, necrosis of plant cells and tissues, etc.

In the process of modulating plant manganese levels, the modified tetracycline inducible cassettes of the present invention may be useful in preventing, detecting, alleviating, and/or conferring resistance to the following, non-limiting, symptoms of manganese deficiency: mottling chlorosis with green veins and leaf web tissue yellow or white on young leaves first, then spreading to old leaves, yellowish green stem, hardening and/or wooding of stem, reduction of carotene, etc.

In the process of modulating plant molybdenum levels, the modified tetracycline inducible cassettes of the present invention may be useful in preventing, detecting, alleviating, and/or conferring resistance to the following, non-limiting, symptoms of molybdenum deficiency: light yellow chlorosis of leaves, failure of leaf blade expansion, etc.

In the process of modulating plant nitrogen levels, the modified tetracycline inducible cassettes of the present invention may be useful in preventing, detecting, alleviating, and/or conferring resistance to the following, non-limiting, symptoms of nitrogen deficiency: stunting plant growth of young plants, yellowish green leaves in young plants, light green leaves in older leaves followed by yellowing and drying or shedding, increased accumulation of anthocyanins in veins, thin stem, spindly appearance of plant, reduced flowering, etc.

In the process of modulating plant phosphorus levels, the modified tetracycline inducible cassettes of the present invention may be useful in preventing, detecting, alleviating, and/or conferring resistance to the following, non-limiting, symptoms of phosphorus deficiency: stunting of young plants, dark blue-green leaves with purplish undertones, slender stems, increased accumulation of anthocyanin in leaves, necrosis of leaves, cessation of meristematic growth, decreased rate of fruit ripening, plant dwarfing at maturity, etc.

In the process of modulating plant potassium levels, the modified tetracycline inducible cassettes of the present invention may be useful in preventing, detecting, alleviating, and/or conferring resistance to the following, non-limiting, symptoms of potassium deficiency: dark green leaves, pale green monocotyledon leaves, yellowing streaking of monocoytledon leaves, marginal chlorosis of leaves, necrosis of leaves appearing first on old leaves, wrinkling of veins, corrugating of veins, crinkling of veins, etc.

In the process of modulating plant sulfur levels, the modified tetracycline inducible cassettes of the present invention may be useful in preventing, detecting, alleviating, and/or conferring resistance to the following, non-limiting, symptoms of sulfur deficiency: light green to yellow leaves appearing first along veins of young leaves, slender stems, etc.

In the process of modulating plant zinc levels, the modified tetracycline inducible cassettes of the present invention may be useful in preventing, detecting, alleviating, and/or conferring resistance to the following, non-limiting, symptoms of zinc deficiency: chlorosis of leaves and/or necrosis of leaves affecting young leaves first, resetting, premature formation of abscission layer of leaves, whitish chlorotic streaks between veins in older laves, whiting of upper leaves in monocotyledons, chlorosis of lower leaves in dicotyledons, etc.

Additional symptoms of plant nutrient deficiencies are known in the art (see for example, Noggle, G. R., and Fritz, G. J., in “Introductory Plant Physiology”, 2^{nd} edition, Prentice-Hall, Inc., Englewood Cliffs, 1983). The modified tetracycline inducible cassettes of the present invention may be capable of preventing, detecting, alleviating, and/or conferring resistance to such symptoms by modulating the gene expression levels of a gene capable of the same.

In another embodiment, the modified tetracycline inducible cassettes of the present invention may be useful in modulating plant nutrient levels by modulating the gene expression of a gene capable of increasing or inducing the secretion of mineral solubilizing or mineral stabilizing compounds or chelating compounds (e.g., citric acid, malic acid, pisidic acid, etc.). Alternatively, the secreted compound may be an organic chelating compound (e.g., phytometallophore, see for example, Cakmak et al., Plant Soil, 180:183-189, (1996)). Alternatively, the secreted compound is a root exudate, such as an organic acid (e.g., lactic, acetic, formic, pyruvic, succinic, tartaric, oxalic, citric, isocitric, aconitic, etc.), carbohydrate, amino acid, or polysaccaride capable of assimilating carbon (see, for example, Paul, E. A., and Clark, F. E., in “Soil microbiology and biochemistry”, Academic Press, San Diego, (1989)).

In another embodiment, the modified tetracycline inducible cassettes of the present invention may be useful in modulate plant nutrient levels by modulating the gene expression of phosphatase enzymes, nitrate reductase enzymes, citrate synthesis enzymes, etc.

In another embodiment, the modified tetracycline inducible cassettes of the present invention may be useful in modulating lant nutrient levels by modulating the gene expression of genes involved in the active transport and/or passive transport mechanisms of the plant. Alternatively, the modified tetracycline inducible cassettes of the present invention may be useful in modulating plant nutrient levels by modulating the expression of genes responsible for inter- and intra-tissue and/or cellular transport of nutrients in the plant (e.g., transport through the phloem, xylem, desmosomes, etc.). Additional mechanisms of modulating plant nutrient transport are known in the art (see, for example, Lambers, H., et al., in “Plant Physiological Ecology”, Spinger-Verlag, New York, (1998); which is hereby incorporated herein by reference in its entirety).

### Antisense Mediated Down-Regulation of Proteins

In preferred embodiments, the invention encompasses the use of the modified tetracycline inducible cassettes of the present invention to modulate the expression of antagonists that correspond to polynucleotide sequences of genes in an antisense orientation. The expression of such antisense polynucleotide sequences would enable investigators to ascertain the biological function of a protein by analyzing the resulting phenotype of a transfected plant under induced and/or non-induced conditions, for example. Moreover, the ability to ascertain the biological function of a protein would be enhanced in the instance where the gene of interest performs a vital function in the plant.

In addition, by using a modified tetracycline inducible cassette of the present invention to modulate the expression of an antagonist corresponding to the polynucleotide sequence of a gene in an antisense orientation, it would be possible to modulate, and/or completing eliminate, the endogenous expression of a particular gene of interest. Such antisense modulation, when coupled to using a modified tetracycline inducible cassette of the present invention to modulate the expression of the same polynucleotide sequence in the sense orientation, would provide complete control of the gene expression for that particular protein.

Antisense technology results in modulation (i.e., complete or partial inhibition), of the expression of a particular protein through direct inhibition of the proteins mRNA. Antisense nucleic acids may be in the form of DNA, RNA, triple helix, quad helix, a chimeric mixture of any of these aforementioned types (e.g., DNA:RNA, etc.), and may be single or double stranded. Antisense nucleic acids modulate gene expression by binding to the RNA of the gene of interest, effectively inhibiting translation. Such interactions may rely follow typical Watson-Crick base pair recognition, or the case of a triple or quad helix, may rely upon Hoogsteen basepair recognition.

The antisense nucleic acids may be transiently generated within the organism (e.g., sequence contained within a modified tetracycline inducible cassette of the present invention introduced into the cells of an organism), stably generated within the organism (e.g., sequence contained within a modified tetracycline inducible cassettes of the present invention introduced into the cells of an organism using transgenic methods, including viral integration, etc.) or may be exogenously administered. For a nucleic acid to serve an antisense role, it is only necessary that it has sequence homology to the sense RNA product of the gene of interest.

A number of methods of administering antisense nucleic acids, their compositions, and designs are known in the art and encompassed by the invention (see for example, Agrawal S, et al., Mol Med Today. 2000 February; 6(2):72-81; Yacyshyn B R, et al, Can J Gastroenterol. 1999 November; 13(9):745-51; Mrsny R J., J Drug Target. 1999;7(1):1-10; Toulme J J, et al, Nucleic Acids Symp Ser. 1997; (36):39-41.), Okano, Neurochem., 56:560 (1991); Oligodeoxynucleotides as Antisense Inhibitors of Gene Expression, CRC Press, Boca Raton, Fla. (1988); and Cooper S R, et al., Pharmacol Ther. 1999 May-June; 82(2-3):427-35). Likewise, a number of methods have been developed regarding the application of triple helix antisense technology to modulating gene expression (see, for example, Gowers D M, et al, Nucleic Acids Res. 1999 Apr. 1; 27(7):1569-77; and Chan P P, et al., J Mol Med. 1997 Apr; 75(4):267-82).

Antisense technology has wide-ranging applications in plants. For example, antisense RNA has been shown to effectively down regulate a variety of plant genes as described by Shimada, et al., Theor. Appl. Genet., 86:665-672, (1993); Kull, et al., J. Genet. Breed., 49:67-76, (1995)., Slabas and Elborough, WO 97/07222; Knutzon et al., Proc. Natl. Acad. Sci. USA, 89:2624-2628, (1992), and Baulcombe D C., Plant Mol Biol. 1996 October; 32(1-2):79-88).

The antisense nucleic acids modulated by the modified tetracycline inducible cassettes of the invention comprise a sequence complementary to at least a portion of an RNA transcript of a gene of interest. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded antisense nucleic acids of the invention, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with a RNA sequence of the invention it may contain and still form a stable duplex (or triplex as the case may be). One skilled in the art can ascertain a tolerable degree of mismatch by use of standard procedures to determine the melting point of the hybridized complex.

Antisense oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs, as well. See generally, Wagner, R., Nature, 372:333-335 (1994). Thus, oligonucleotides complementary to either the 5′- or 3′-non-translated, non-coding regions of a polynucleotide sequence of the invention could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

In a specific embodiment, the modulated antisense nucleic acid comprises catalytic RNA, or a ribozyme (see, e.g., PCT International Publication WO 90/11364, published Oct. 4, 1990; Sarver et al., 1990, Science 247:1222-1225; Hasselhoff, et al., Nature 342:76-79 (1988)). Ribozymes have been used to down regulate gene expression, and more recently in the down regulation of plant proteins (seem e.g., PCT International Publication WO 97/10328). In another embodiment, the oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

### Antibody Mediated Down-Regulation of Proteins

The modified tetracycline inducible cassettes of the present invention may be useful in modulating specific characteristics of a plant, such as endogenous traits, growth and differentiation, stress tolerance, and other traits or characteristics known in the art and described elsewhere herein, by modulating the expression of antibody genes encoding antibodies directed against proteins integral to these traits. For example, the modulated antibody genes may be directed against endogenous plant proteins, pathogenic proteins (i.e., pathogen encoded proteins required for permissive infection), endogenous proteins required for permissive pathogen infection (e.g., receptors, etc.), etc.

Alternatively, the modified tetracycline inducible cassettes of the present invention are useful in determining the function of a plant protein where the cassettes are used to modulated the gene expression of an antibody gene encoding an antibody directed against a protein of interest. Thus, by inhibiting the expression of a protein in a plant, coupled with observations of its resulting phenotype under induced, or non-induced conditions, a function of the protein may be assigned.

In addition, by using a modified tetracycline inducible cassette of the present invention to modulate the expression of an antagonist corresponding to the polynucleotide sequence of an antibody gene encoding an antibody directed against a protein of interest, it would be possible to modulate, and/or completing eliminate, the endogenous expression of a particular protein of interest. Such antibody-mediated modulation, when coupled to using a modified tetracycline inducible cassette of the present invention to modulate the expression of the polynucleotide sequence encoding the same protein of interest, would provide complete control of the gene expression for that particular protein.

The method of modulating endogenous gene expression using antibodies is disclosed in International Publication Number WO 00/05391, which is hereby incorporated in its entirety herein. In this example, the researchers were able to achieve 40-70% inhibition of an endogenous plant protein through the use of a single-chain antibody gene directed against the plant protein. The method is directed towards the production of monoclonal antibodies, specifically, single chain antibodies, specific to endogenous transit peptides in a plant in an effort to decrease steady state levels of such transit peptides within the plant. The method is comprised of the following steps: I) generating monoclonal antibodies to a specific protein, II) cloning the gene for said monoclonal antibody, III) creating an expression vector comprising a fusion of the heavy-chain and light chain gene sequences of said monoclonal antibody gene downstream of the p67 leader peptide, and under the control of one of the modified tetracycline inducible cassettes of the present invention, IV) optimizing the codons of said heavy-chain and light chain fusion vector for efficient expression of the gene encoded thereof in a plant, and V), transfecting a plant with said heavy-chain and light chain fusion expression vector.

The skilled artisan would appreciate the methods described therein (WO 00/05391), and would have the ability to apply such methods to the modified tetracycline inducible cassettes described herein. The artisan would appreciate that such a cassette could be useful in inhibiting the steady-state expression levels of any of the polypeptides referred to herein and/or known in the art, including variants, and fragments, thereof. The skilled artisan would appreciate that any leader peptide (i.e., signal sequence) from a plant protein could be used in creating the heavy-chain and light chain fusion vector. The skilled artisan would also appreciate that different plant species may have different codon usage requirements, and thus, the decision to optimize the codons of the heavy-chain and light chain fusion vector would be affected according to the codons required for that particular plant species.

The method could not only be applied to transit peptides, but also to secreted proteins, membrane proteins, receptors, and ligands. The method could also be applied in combination with other antibody production methods in plants. For example, antibodies directed towards polypeptides of the present invention may inhibit specific traits in a plant which could increase the plants defense mechanisms to pathogens. Thus, where such an antibody was expressed, another antibody could be expressed in combination with the first, to inhibiting the pathogenicity of a plant pathogen by directing the expression of antibodies directed towards pathogenic proteins (e.g., those proteins critical to the initiating events of infection, such as the BUF1 gene from M. grisea, stage two juvenile salivary gland proteins which include, svp30, scp31a, scp31b, scp32, scp32, scp39, and scp49 from G. rostochiensis (WO 96/22372), etc.). Such a combination would also be of value where the second “anti-pathogenic” antibody is an antibody directed towards a pathogen and fused to a toxic protein wherein such a toxin could be chitinase, glucanase, lysozyme, BT, or colicin F, for example (see WO 96/09398), etc.).

As described elsewhere herein, the method could also be used as a means of inhibiting allergic reactions to plant antigens in humans, mammals, animals, etc., by directing the production of a single chain antibody protein specific towards said plant antigen in the plant (via transgenic methodology). In the latter example, the plant would not be limited to edible plants, as inhibiting the production of such a plant antigen would provide benefit to a human by removing the antigen from the humans environment, for example, irrespective of whether the plant was ingested.

Of particular interest, is the fact that secretion of functional antibody through the plasma membrane of plant cells has been reported for protoplasts isolated from transgenic plants and for callus cells adapted to suspension culture (Hein et al., Biotechnol. Prog. 7:455-561, 1991). However, the levels of secreted antibody detected in both culture systems were extremely low. In other studies, cultured tobacco cells were transformed with a gene encoding a synthetic antibody derivative expressed as a single chain consisting of both the heavy- and light-chain variable domains of the intact immunoglobulin joined together by a flexible peptide linker (Pluckthun, Immunol. Rev. 130:151-188, 1991; and Bird et al., Science 242:423-426, 1988). This synthetic single-chain antibody retained the full antigen-binding potential of the intact immunoglobulin but accumulated in the extracellular apoplastic space of the transformed cells (Firek et al., Plant Molecular Biology 23:861-870, 1993), indicating that the antibody was being transported through the plasma membrane but not through the cell wall to the external environment. Moreover, recent studies have shown that increased antibody production in a plant, and heterologous protein expression, in general, could be increased by including in the plant culture medium a protein stabilizing agent (e.g., polyvinylpyrrolidone), see U.S. Pat. No. 6,020,169, which is hereby incorporated by reference in its entirety herein.

### Uses for the Tetracycline Analogs/Functional Equivalents Identified by the Present Invention

The tetracycline analogs and/or functional equivalents identified by the present invention may be used as a substitute for tetracycline, any currently known tetracycline analog, and/or any known analogs functional equivalents. The tetracycline analog and/or equivalent may have inherent properties that make it advantageous to its application in a tetracycline inducible system. For example, when compared to tetracycline, known tetracycline analogs, or known tetracycline functional equivalents, the tetracycline analog and/or functional equivalent of the present invention may be more soluble, have increased photo-stability, have increased thermal-stability, may be less toxic to cells and tissues, may have increased affinity for tet repressor, have a decreased equilibrium dissociation constant with tet repressor, may have more favorable induction kinetics, may have a more favorable mode of induction, have increased thermodynamic interaction with tet repressor, etc. By “tetracycline functional equivalent” is meant a molecule that is capable of binding to the tet repressor in a manner that enables the molecule to modulate the ability of the tet repressor to bind to its operator sites (e.g., a molecule capable of dissociating tet repressor from a tet operator, a molecule capable of derepressing a tetracycline inducible system, a molecule capable of repressing a tetracycline inducible system, etc.). Such a molecule may, or may not, share structural characteristics of tetracycline molecules known in the art. Alternatively, when compared to tetracycline, known tetracycline analogs, or known analogs functional equivalents, the tetracycline analog and/or functional equivalent of the invention may be less soluble, have decreased photo-stability, have decreased thermal stability, have decreased thermal stability, be more toxic to cells and tissues, have decreased affinity for tet repressor, have an increased equilibrium dissociation constant with tet repressor, may have less favorable induction kinetics, may have decreased thermodynamic interaction with tet repressor, etc. The tetracycline analog and/or equivalent of the invention may have any combination of the above characteristics.

### References

Barkley, M. D. and Bourgeois, S. 1980. In: “The Operon”. Miller, J. H. and Reznikoff, W. S. (eds), Cold Spring Harbor Laboratory Press, Cold Spring Harbor, N.Y., pp. 177-220.
Boulikas, T. 1993. Nuclear Localization Signals (NLS). Critical Reviews in Eukaryotic Gene Expression, 3(3), pp. 193-227.
Beck, C. F., Mutzel, R., Barbe, J., and Muller, W. 1982. A multifunctional gene (tetR) controls Tn10-encoded tetracycline resistance. Journal of Bacteriology, May 1982, pp. 633-642.
Degenkolb, J., Takahashi, M., Ellestad, G. A., & Hillen, W. 1991. Structural requirements of tetracycline-tet repressor interaction: Determination of equilibrium binding constants for tetracycline analogs with the tet Repressor. Antimicrobial Agents Chemotherapy, vol. 35, No 8, pp. 1591-1595.
Deuschle, U., Meyer, W. K. H. and Thiesen, H. J. (1995). tetracycline-reversible silencing of eukaryotic promoters. Mol. Cell. Biol. 15, 1907-1914.
Furth, P., Onge, L., Boger, H., Gruss, P., Gossen, M., Kistner A., Bujard, H. & Henninghausen, L. (1994). Temperal control of gene expression in transgenic mice by a tetracyclin-responsive promoter. Proc. Natl. Acad. Sci. USA 91, 9032-9306.
Gatz, C., Kaiser, A., and Wendenburg, R. 1991. Regulation of a modified CaMV 35S promoter by the Tn10-encoded tet repressor in transgenic tobacco. Mol. Gen. Genet., 227, pp. 229-237.
Gatz, C., Frohberg, C., and Wendenburg, R. 1992. Stringent represion and homogeneous de-repression by tetracycline of a modified CaMV 35S promoter in intact transgenic tobacco plants. The Plant Journal, 2(3), pp. 397-404
Gossen, M. and Bujard, H. (1992). Tight control of gene expression in mammalian cells by tetracycline-responsive promoters. Proc. Natl. Acad. Sci. USA 89, 5547-5551.
Gossen, M., Freundlieb, S., Bender, G., Muller, G., Hillen, W., and Bujard, H. 1995. Transcriptional activation by tetracyclines in mammalian cells. Science, vol. 268, pp. 1766-1769.
Hall, G. E., Allen, G. C., Loer, D. S., Thompson, W. F., and Spiker, S. 1991. Nuclear scaffolds and attachment regions (SARs) in higher plants. PNAS, 88, 9320-9324.
Jefferson, R. A. 1987. Assaying chimeric genes in plants: the GUS gene fusion system. Plant Molecular Biology Reports 5, 387-405.
Jones, H., Ooms, G., Jones, M. G. K. 1989. Transient gene expression in electroporated Solanum protoplasts. Plant Molecular Biology, 13, pp. 503-511.
Kao and Michayluk. 1975. Nutritional requirements for growth of Vicia hajastana cells at very low population density in liquid medium. Planta 126:105-110.
Lederer, T., Kintrup, M., Takahashi, M., Sum, P. E., Ellestad, G. A., and Hillen, W. 1996. tetracycline analogs affecting binding to Tn10-encoded tet Repressor trigger the same mechanism of induction. Biochemistry 35, 7439-7446.
Murashige, T. and Scoog, F. 1962. A revised medium for rapid growth and bioassays with tobacco tissue cultures. Physiologia Plantarum, 15, 473-497.
Negrutiu, I., Shillito, R., Potrykus, I., Biasini, G. and Sala, F. 1987. Hybrid genes in the analysis of transformation conditions. Plant Molecular Biology, 8, 363-373.
Roder, F. T., Schmulling, T., and Gatz, C. 1994. Efficiency of the tetracycline-dependent expression system: complete suppression and efficient induction of the rolB phenotype in transgenic plants. Mol. Gen. Genet., 243:32-38.
Rogalski, W. 1985. The tetracyclines. Hlavka, J. J., and Boothe, J. H., Eds., Springer-Verlag, Heidelberg, Germany, pp 179-326.
Rosahl, S., Schmidt, R., Schell, J. and Willmitzer, L. 1987. Expression of a tuber-specific storage protein in transgenic. tobacco plants: demonstration of an esterase activity. EMBO J., 6, 1155-1159.
Vervliet, G., Holsters, M., Teuchy, H., Van Montagu, M. and Schell, J. 1975. Characterization of different plaque-forming and defective temperate phages in Agrobacterium strains. J. Gen Virol. 26, 33-48.
Ulmasov, B., Capone, J., and Folk, W. 1997. Regulated expression of plant tRNA genes by the procaryotic tet and lac repressors. Plant Molecular Biology, 35:417-424.
Sathasivan et al. 1990. Nucleotide sequence of a mutant acetolactate synthase gene from an imidazolinone-resistant Arabidopsis thaliana var. Columbia. Nucl. Acids Res., 18, p. 2188.
Wilde, R. J., Shufflebottom, D., Cooke, S., Jasinska, I., Meryrweather, A., Beri, R., Brammar, W. J., Bevan, M., and Schuch., W. 1992. Control of gene expression in tobacco cells using a bacterial operator-repressor system. The EMBO journal, vol. 11, No. 4, pp. 1251-1259.
Wirtz, E.and Clayton, C. 1995. Inducible gene expression in trypanosomes mediated by prokaryotic repressor. Science, vol. 268, pp. 1179-1183.
Wray, L. V. Jr., and Reznikoff, W. S. 1983. Identification of repressor binding sites controlling expression of tetracycline resistance encoded by Tn10. Journal of Bacteriology, December 1983, p. 1188-1191.

Throughout the disclosure of the present invention, numerous references have been cited which include, for example, publications, journal articles, issued US patents, published PCT application, European patent applications, etc. The disclosure of such references cited herein should be construed as an explicit incorporation by reference of the material in its entirety herein.

Having generally described the invention, the same will be more readily understood by reference to the following examples, which are provided by way of illustration and are not intended to be limiting.

### EXAMPLES

### Example 1

### Method of Creating the Novel Tetracycline Repressor, Operator, Repressor/Operator Cassettes, and Vectors of the Present Invention

Sources for Gene Elements

Coding region for wild type tet Receptor, as well as TripleX promoter were obtained from Christiane Gatz (Pflanzenphysiologisches Institut, Universiat Gottingen, Untere Karsphle 2, D-37073 Gottingen, Germany).
SV40 Nuclear Localization Sequence was available in public domains (for reference see Boulikas, 1993).
Coding region for GUS gene was licensed from Center for the Application of Molecular Biology to International Agriculture, GPO Box 3200, Camberra, ACT 2601, Australia.
Arabidopsis thaliana AHAS gene with an imidazolinone specific resistance mutation site at amino acid position 653 (Sathasivan et al., 1991), Arabidopsis thaliana HPPD and Arabidopsis thaliana AHAS promoters were isolated and cloned by Cyanamid.
Original vector for Agro transformation, pCAMBIA2300 and pCAMBIA3300, were purchased from Center for the Application of Molecular Biology to International Agriculture, GPO Box 3200, Camberra, ACT 2601, Australia.
Coding region for Firefly Luciferase (pGL3basic) was purchased from Promega Corporation, 2800 Woods Hollow Rd., Madison, Wis. 53711-5399.
Coding region for Renilla Luciferase (pRLnull) was purchased from Promega Corporation, 2800 Woods Hollow Rd., Madison, Wis. 53711-5399.
Vectors for cloning basic elements, pBCKS(−) and PBSIIKS+, were purchased from Stratagene Inc., 11011 North Torrey Pines Road, La Jolla, Calif. 92037.
CaMV 35S promoter was publicly available.
Arabidopsis thaliana Actin promoter was licensed from Richard B. Meagher, Department of Genetics, University of Georgia, Athens, Ga. 30602.
(OCS)₃MAS promoter was licensed from Stanton B. Gelvin (Department of Biological Sciences, Purdue University, West Lafayette, Ind. 47907, USA).
OCS element is derived from Agrobacterium tumefaciens.
Arabidopsis Hppd and AHAS promoters were isolated and cloned by Cyanamid.
1100 bp RB7 MAR in pRB7-6 was licensed from Steven Spiker, Department of Genetics, North Carolina State University, Raleigh, N.C. 27695.

### Procedures

Background Information: How Basic Elements were Transferred from Original Source to Convenient Vectors

Vectors for Cloning.

pACGH010. Produced after ligation of pBCKS(−) Kpn/Sac and MCS1a (annealed MCS1a/MCS1ar) and MCS1b (annealed MCS1b/MCS1br), each fragment was ˜70 bp.

pACGH011. Produced after ligation of pBCKS+ Kpn/Sac and MCS1a (annealed MCS1a/MCS1ar) and MCS1b (annealed MCS1b/MCS1br) each fragment was ˜70 bp.

Coding Regions.

NLS-tetReceptor Coding Region.

Wild type tet Receptor was obtained from Christiane Gatz, supra, and cloned into Cyanamid plasmid, pAC448. pACDV001 was made after ligation of vector, pGEM3ZfP, cut with BamHI and XbaI, and insert, 620 bp fragment of pAC448 cut with BamHI and XbaI. pACGH004 was made after ligation of vector, pACDV001, cut with XbaI, and insert, 20 bp SV40NLS fragment produced by annealing two complementary oligonucleotides. The NLS-tetReceptor coding region (640 bp) was cloned into pACGH011 to yield pACAG001 that was later supplemented with NOS terminator to produce pACAG006.

MAR

Originally was available in pRB7-6 (Spiker, supra). pACGH005 was created after 1168 bp ClaI/ScaI fragment from the pRB7-6 was cloned into pBCKS (−) cut with ClaI and SmaI. The (OCS)₃MAS/nTR cassette was cloned into pACGH016 between two copies of this element to produce pACAG021.

Renilla Coding Region.

Originally was available in pRLnull (Promega, supra). pACGH038 was created after ligation of pRLnull cut with SphI and SalI and CIAP and 780 bp fragment of pCAMB3300 cut with BstXI and XhoI (35S promoter). After several rounds of subcloning the 935 bp fragment coding region was cloned into pACGH042 as a cassette and, later, into pACRS012 as a vector for dual luciferase assay.

Luciferase Coding Region.

Originally was available in pGL3basic (Promega, supra). pACGH040 was created after ligation of pACGH011 cut with StuI and 1650 bp of pGL3basic cut with NcoI and XbaI and Klenow-treated. After several rounds of subcloning the Luciferase coding region was inserted into pACRS012 as a part of an expression cassette for dual luciferase assay.

AHAS Coding Region.

The AtAHAS (S653N) (Sathasivan, supra). pAC321 was made after ligation of the 5710 bp fragment containing the AtAHAS (S653N) XbaI into BlueSKp XbaI. Full insert sequence was determined by ACGT Inc. pACGH044 was made after ligating vector, pACGH011 cut with StuI and CIP-treated, and insert, ˜2000 bp fragment of pAC321 cut with NcoI and AgeI and Klenow-treated.

NPTII cassette was available in pCAMBIA2300 (Cambia, supra).

Promoters.

TripleX (CaMV 35S promoter with three tet operators).

Originally was available in pUCA7-TX, the construct obtained from Christine Gatz. After several rounds of subcloning the promoter appeared in pAC446. The 550 bp EcoRI and HindIII fragment from pAC446 was blunt-ended with Klenow and ligated into pACGH010 HpaI/CIP to yield pACGH062.

CaMV 35S Promoter.

The 430 bp BamHI/XbaI fragment from pAC401 was blunt-ended with T4 polymerase and ligated into pACGH010 HpaI/CIP to yield pACGH061.

(OCS)₃MAS Promoter.

The 1203 bp SalI/XbaI fragment from pAC154-2 was blunt-ended with Klenow and ligated to pACGH010 HpaI/CIP to yield pACRS002.

Double Enhanced CaMV 35S Promoter.

Originally was available in pCAMBIA3300. After several rounds of subcloning the promoter appeared in pACGH041. pACGH041 was digested with NheI/HindIII. The 800 bp fragment was gel isolated, treated with Klenow and ligated to pACGH010 HpaI digested, CIP treated to yield pACGH046.

Arabidopsis Hppd Promoter.

797 bp NcoI/KpnI fragment from pAC1541, blunt ended with mungbean nuclease and ligated to pACGH010 HpaI/CIP to yield pACGH056.

Arabidopsis Ahas Promoter.

The 2483 bp NotI/NcoI fragment from pAC321 was blunt-ended with mungbean nuclease and ligated to pACGH010 HpaI/CIP to yield pACGH057.

Arabidopsis Actin Promoter

pWACT2S was digested with BbvI, mungbean nuclease treated and gel purified. This fragment was digested with SalI to release 1450 bp fragment which was treated with Klenow and ligated to HpaI digested pACGH010 to yield pACRS031.

### Description of Cassette Construction

pACAG013 Vector: pACGH044 cut with FseI and AscI and CIP-treated; insert: 780 bp fragment of pACGH022 cut with FseI and AscI.
pACAG015 Vector: pACAG006 cut with Sse83871 and NotI and CIP-treated; insert: 1240 bp fragment of pACRS002 cut with Sse83871 and NotI.
pACAG023 Vector: pACAG013 cut with PstI and NotI and CIP-treated; insert: 580 bp fragment of pACGH062 cut with PstI and NotI.
pACAG024 Vector: pACRS012 cut with EcoRI and PstI and CIP-treated; insert: 580 bp fragment of pACGH062 cut with EcoRI and PstI
pACAG029 Three-way ligation. Vector: pACGH113 cut with AscI and AvrII, CIP-treated; insert 1: 3390 bp fragment of pACAG023 cut with AscI and NotI, insert 2: 2270 bp fragment of pACAG015 cut with AvrII and NotI.
pACAG033 Vector: pACRS002b cut with PstI and AscI, CIP-treated; insert: 580 bp fragment of pACGH062a cut with PstI and AscI.
pACAG042 Vector: pACRS012 sequentially treated with PstI, T4 Polymerase, NotI, and CIP; insert: 1400 bp fragment of pACAG033 sequentially treated with SphI, T4 Polymerase, and NotI.
pACAG048 Vector: pACRS002 sequentially treated with PstI, T4 Polymerase, AvrII, and CIP; insert: 140 bp fragment of pACGH062 cut with EcoRV and AvrII.
pACAG049 Vector: PCAMBIA2300 cut with SstI, CIP-treated; insert: 4680 bp fragment of pACAG021 cut with SstI.
pACAG050 Vector: pACRS012 sequentially treated with PstI, T4 Polymerase, NotI, and CIP; insert: 950 bp fragment of pACAG048 cut with SphI, T4-treated, cut with NotI
pACAG066 Vector: pACAG015 cut with NotI and Sse83871, CIP-treated; insert: 730 bp fragment of pACGH046 cut with NotI and Sse83871.
pACAG067. Vector: pACAG015 cut with NotI and Sse83871, CIP-treated; insert: 800 bp fragment of pACGH056 cut with NotI and Sse83871.
pACAG068 Vector: pACAG015 cut with NotI and Sse83871, CIP-treated; insert: 2480 bp fragment of pACGH057 cut with NotI and Sse83871.
pACAG069 Vector: pACAG015 cut with NotI and Sse83871, CIP-treated; insert: 430 bp fragment of pACGH061 cut with NotI and Sse83871.
pACAG070 pACGH046 was cut with MluI and EcoRV, Klenow-treated and re-circularized. As a result, 670 bp fragment was removed from the vector.
pACAG071 pACRS002 cut with XhoI and MluI, Klenow-treated and re-circularized. As a result, 820 bp fragment was removed from the vector.
pACAG073 Vector: pACAG064 cut with KpnI, and CIP-treated; insert: 2530 bp fragment of pACAG024 cut with KpnI.
pACAG074 Vector: pACRS012 cut with EcoRI, PstI, and CIP-treated; insert: 186 bp fragment of pACAG070 cut with EcoRI and PstI.
pACAG075 Vector: pACRS012-cut with EcoRI, PstI, and CIP-treated; insert: 440 bp fragment of pACAG070 cut with EcoRI and PstI
pACAG076 Vector: pACGH113 cut with KpnI, CIP-treated; insert: 1460 bp fragment of pACAG069 cut with KpnI.
pACAG077 Vector: pACGH113 cut with KpnI, CIP-treated; insert: 1841 bp KpnI fragment from pACAG066.
pACAG078 Vector: pACGH113 cut with KpnI, CIP-treated; insert: 1830 bp fragment of pACAG067 cut with KpnI.
pACAG079 Vector: pACGH113 cut with KpnI, CIP-treated; insert: 3516 bp fragment of pACAG068 cut with KpnI.
pACAG081 Vector: pACAG064a cut with KpnI, and CIP-treated; insert: 2530 bp fragment of pACAG024 cut with KpnI.
pACAG083 Vector: pACGH011 cut with SacII and Apai, CIP-treated; insert: 2900 bp fragment of pACAG050 cut with SacII and ApaI.
pACAG084 Vector: pACGH113 cut with KpnI, CIP-treated; insert: 2243 bp fragment of pACAG015 cut with KpnI.
pACAG085 Vector: pACAG084 cut with KpnI, and CIP-treated; insert: 2530 bp fragment of pACAG024 cut with KpnI.
pACAG086 Vector: pACGH010 cut with PstI, MluI and CIP-treated; insert: 105 bp PCR product of amplification of pACRS002 with primers F2M and R2 digested with PstI and MluI.
pACAG087 Vector: pACGH010 cut with PstI, MluI and CIP-treated; insert: 200 bp PCR product of amplification of pACGH061 with primers F61M and R6122 digested with PstI and MluI.
pACAG088 Vector: pACRS012 cut with EcoRI, PstI, and CIP-treated; insert: 186 bp fragment of pACAG086 cut with EcoRI and PstI.
pACAG089 Vector: pACRS012 cut with EcoRI, PstI, and CIP-treated; insert: 186 bp fragment of pACAG087 cut with EcoRI and PstI.
pACAG093 Vector: pACAG087 sequentially treated with MluI, Klenow, SacII, and CIP; insert: 777 bp fragment of pACRS002 sequentially treated with PstI, T4 Polymerase, and SacII.
pACAG094 Vector: pACAG086 sequentially treated with MluI, Klenow, SacII, and CIP; insert: 777 bp fragment of pACRS002 sequentially treated with PstI, T4 Polymerase, and SacII.
pACAG095 Vector: pACRS012 cut with MluI and PstI, CIP-treated; insert: 990 bp fragment of pACAG093 cut with MluI and PstI.
pACAG096 Vector: pACRS012 cut with MluI and PstI, CIP-treated; insert: 890 bp fragment of pACAG094 cut with MluI and PstI.
pACAG098 Vector: pACAG066 cut with NotI and Sse83871, CIP-treated; insert: 1450 bp fragment of pACRS031 cut with NotI and Sse83871.
pACAG105 and 105r Vector: pCAMBIA2300 cut with KpnI, CIP-treated; insert: 2090 bp fragment of pACAG088 cut with KpnI.
pACAG106 and 106r Vector: pCAMBIA2300 cut with KpnI, CIP-treated; insert: 2190 bp fragment of pACAG089 cut with KpnI.
pACAG107r Vector: pCAMBIA2300 cut with KpnI, CIP-treated; insert: 2960 bp fragment of pACAG095 cut with KpnI.
pACAG108r Vector: pCAMBIA2300 cut with KpnI, CIP-treated; insert: 2860 bp fragment of pACAG096 cut with KpnI.
pACAG109 Vector: pCAMBIA2300 cut with KpnI, CIP-treated; insert: 2450 bp fragment of pACAG098 cut with KpnI.
pACAG112 Vector: pCAMBIA013 cut with NotI & PstI, CIP-treated; insert: 906 bp fragment of pACAG048 cut with NotI & PstI.
pACAG113 Vector: pACAG084 cut with KpnI, and CIP-treated; insert: 2860 bp fragment of pACAG083 cut with KpnI.
pACAG119 Vector: pACAG109 cut with KpnI, CIP-treated; insert: 3706 bp fragment of pACAG112 cut with KpnI.
pACAG119r The same as pACAG119, only different mutual orientation of cassettes.
pACAG120 Vector: pACAG109r cut with KpnI, CIP-treated; insert: 3706 bp fragment of pACAG112 cut with KpnI.
pACAG120r The same as pACAG120, only different mutual orientation of cassettes.
pACAG124 Vector: pACAG109 cut with KpnI, CIP-treated; insert: 2860 bp fragment of pACAG083 cut with KpnI.
pACRS018 Vector: pACGH054 cut with EcoRI and PstI, CIP-treated; insert: 470 bp fragment of pACGH061 cut with EcoRI and PstI.
pACGH113 93 bp HindIII/EcoRI fragment from pACGH011b ligated to pCAMBIA2300 digested with HindIII/EcoRI, CIP-treated.
pAC489. Wild type tet receptor received from Christine Gatz was cloned into high copy vector between 35S promoter and OCS terminator to yield pAC448. pAC448 carrying 35S promoter-TET repressor gene-OCS terminator was digested with EcoRI and HindIII. The ˜1.4 kb 35S promoter-TET repressor gene-OCS terminator was isolated. pFFF19k was put into PCR with primers cam5pf, forward primer which adds an upstream HindIII, and NPTBam which adds a BamHI site at 3′ end of NPTII. The PCR product was digested with HindIII and BamHI for generating cohesive ends for subsequent 3 way ligation, and HindIII-35S promoter-NPTII-BamHI product was isolated. pAC449 was cut with EcoRI and BamHI. The ˜10 kb pBIN backbone with the pAg2 terminator was taken. The three fragments were put in a triple ligation, cells were transformed and screened.
pAC499. pUCA7-TX carrying 35S CaMV promoter containing three TET-Repressor binding sites (triple X) was received from Christiane Gatz. EcoRI and HindII fragment of this plasmid was transferred into a vector to yield pAC446. pAC447 was digested with EcoRI and HindIII and the ˜3 kb E-triplex promoter-GUS-OCS-H fragment was isolated. pAC was digested with EcoRI and BamHI and the ˜10 kb pBIN backbone EB-pAg7 fragment was isolated. pAC498 was cut with HindIII and BamHI, the ˜1.6 kb HindIII-35S promoter-HPH-35S terminator-BamHI fragment was isolated. The three fragments were ligated and competent cells were transformed and screened.

### 35S-/MAS-Based Promoter Cassettes

Oligonucleotides for promoter elements were ordered from Genosys Biotechnologies, 1442 Lake Front Circle, Suite 185, The Woodlands, Tex. 77380.

The following oligonucleotide pairs were annealed to yield fragments with protruding ends complimentary with PstI restriction site ends (see FIG. 31):

| | |
|---|---|
| tet operator fragment | |
| tetO-f: CTCTATCAGTGATAGAGTCTGCA | (SEQ ID NO:24) |
| tetO-r: GACTCTATCACTGATAGAGTGCA | (SEQ ID NO:25) |
| 35S fragment | |
| 35Sprom-f: ATTTGGAGAGGACACGCTGCA | (SEQ ID NO:26) |
| 35Sprom-r: GCGTGTCCTCTCCAAATTGCA | (SEQ ID NO:27) |
| tet operator + | |
| MAS tata fragment | |
| tetO-tata-f: | |
| CTCTATCAGTGATAGAGTTATTATATCTGCA | (SEQ ID NO:28) |
| tetO-tata-r: | |
| GATATAATAACTCTATCACTGATAGAGTGCA | (SEQ ID NO:29) |
| MAS caat-tata | |
| fragment | |
| caat-tata-f: AAATGGATAAATACTGCA | (SEQ ID NO:30) |
| caat-tata-r: GTATTTATCCATTTTGCA | (SEQ ID NO:31) |
| tet operator + | |
| MAS caat-tata fragment | |
| tetO-caat-tata-f: | |
| CTCTATCAGTGATAGAGTAAATGGATAAATACTGCA | (SEQ ID NO:32) |
| tetO-caat-tata-r: | |
| GTATTTATCCATTTACTCTATCACTGATAGAGTGCA | (SEQ ID NO:33) |

pACAG121 Vector: pACGH010 cut with HpaI; insert: 73 bp PCR product of amplification of pACAG024 with primers F35mlu, 5′-TACGCGTATCTCCACTGACGTA-3′ (SEQ ID NO:34) and Rtrip, 5′-CTTATATACACTCTATCACT-3′ (SEQ ID NO:35). F35mlu is a primer with an 8 bp extension carrying the MluI restriction site. It binds to the 5′-end of both the minimal 35S and minimal TripleX promoters. Rtrip is a 3′-end primer used in generating TripleX truncated promoter.
pACAG123 Three-way ligation. Vector: pACAG024 cut with EcoRV and PstI, CIP-treated; insert 1: 80 bp of pACAG121 cut with MluI, Klenow-treated, cut with PstI; insert 2: 770 bp fragment of pACRS002b cut with PstI, T4-treated, cut with MluI.
pACAG125 Vector: pACGH010 cut with HpaI; insert: 73 bp PCR product of amplification of pACAG069 using F35mlu (SEQ ID NO:34) and R35S, 5′-CTTATATAGAGGAAGGGTCT-3′ (SEQ ID NO:36) pair of primers. The R35S primer is a 3′-end primer used in generating 35S truncated promoter.
pACAG134 Three-way ligation.Vector: pACAG024 cut with EcoRV and PstI, CIP-treated; insert 1: 80 bp of pACAG125 cut with MluI, Klenow-treated, cut with PstI; insert 2: 770 bp fragment of pACRS002b cut with PstI, T4-treated, cut with MluI.
pACAG127 Vector: pACAG123 cut with PstI; insert: one tet operator and two 35S fragments.
pACAG130a Vector: pACAG123 cut with PstI; insert: three tet operator fragments.
pACAG131 Vector: pACAG123 cut with PstI; insert: four tet operator fragments.
pACAG135 Vector: pACAG123 cut with PstI; insert: two tet operator and one 35S fragments.
pACAG137 Vector: pACAG134 cut with PstI; insert: three 35S fragments.
pACAG139 Vector: pACAG134 cut with PstI; insert: two tet operator and one 35S fragments.
pACAG140a Vector: pACAG123 cut with PstI; insert: two tet operator and one 35S fragments.
pACAG141 Vector: pACAG134 cut with PstI; insert: one tet operator and two 35S fragments.
pACAG141a Vector: pACAG134 cut with PstI; insert: two tet operator and two-35S fragments.
pACAG142a Vector: pACAG134 cut with PstI; insert: one tet operator and two 35S fragments.
pACAG150 Vector: pACAG024 cut with PstI and MluI; insert: 770 bp fragment of pACRS002 cut with PstI and MluI.
pACAG163 Vector: pACAG150 cut with PstI; insert: MAS caat-tata and 35S fragments.
pACAG164 Vector: pACAG150 cut with PstI; insert: tet operator+MAS caat-tata and 35S fragments.
pACAG165a Vector: pACAG150 cut with PstI; insert: MAS caat-tata and three tet operator+MAS tata fragments.
pACAG166 Vector: pACAG150 cut with PstI; insert: tet operator+MAS caat-tata and one tet operator+MAS tata fragments.
pACAG151 Vector: pACAG084 cut with KpnI, and CIP-treated; insert: 2860 bp fragment of pACAG127 cut with KpnI.
pACAG152 Vector: pACAG084 cut with KpnI, and CIP-treated; insert: 2860 bp fragment of pACAG131 cut with KpnI.
pACAG153 Vector: pACAG084 cut with KpnI, and CIP-treated; insert: 2860 bp fragment of pACAG135 cut with KpnI.
pACAG154 Vector: pACAG084 cut with KpnI, and CIP-treated; insert: 2860 bp fragment of pACAG137 cut with KpnI.
pACAG155 Vector: pACAG084 cut with KpnI, and CIP-treated; insert: 2860 bp fragment of pACAG139 cut with KpnI.
pACAG156 Vector: pACAG084 cut with KpnI, and CIP-treated; insert: 2860 bp fragment of pACAG141 cut with KpnI.
pACAG157 Vector: pACAG084 cut with KpnI, and CIP-treated; insert: 2860 bp fragment of pACAG141a cut with KpnI.
pACAG168 Vector: pACAG084 cut with KpnI, and CIP-treated; insert: 2860 bp fragment of pACAG130a cut with KpnI.
pACAG169 Vector: pACAG084 cut with KpnI, and CIP-treated; insert: 2860 bp fragment of pACAG140a cut with KpnI.
pACAG170 Vector: pACAG084 cut with KpnI, and CIP-treated; insert: 2860 bp fragment of pACAG142a cut with KpnI.

### Example 2

### General Methods of the Invention

Protoplast Culture

Protoplasts from NT1 cells were isolated accordingly to protocol in Hall, 1991. Mesophyll protoplasts were prepared from tobacco leaves by a method of Negrutiu et al (1987).

Electroporation of Protoplasts with High Copy Number Vectors.

Exponential decay pulses were generated by a Gene Pulser apparatus (Bio-Rad Laboratories, Richmond, Calif.) set at 960 μF and 0.45 kV; 0.4 cm electrode gap potter-type cuvettes (Bio-Rad) were used. An aliquot containing 2.5×10⁶ cells was electroporated with 15-20 μg of one or two plasmids accordingly to protocol described by Jones et al (1989). After electroporation cells were resuspended in NT1 medium supplemented with mannitol 0.4M and cultivated on the shaker at 90 rpm and 27° C. with a light intensity of 47 μmol m⁻²sec⁻¹.

Seed sterilization and germination. All seeds have been vernalized (kept at +40° C.) for at least a week prior their introduction into culture. 1) Tobacco seeds were washed with 100% ethanol for 2 minutes, dried, and transferred to plates with appropriate media. 2) Arabidopsis seeds were sterilized as follows:
Wash with ethanol 70% for 5 min;
Three washes with solution containing 50% Chlorox and 0.1%
Triton x100, 10 min each;
Three washes with sterile water, 5 min each.

Seeds were dried and transferred to plates with appropriate media.

Production of Agrobacterium strains.

Agro vectors were introduced into Agrobacterium tumefaciens strain LBA4404 using heat shock technique.
Add 2 ml of overnight Agrobacterium culture to 50 ml YEB medium and shake at 250 rpm and 28° C. until the culture growth to an OD600 of 0.5 to 1.0.
Chill the culture on ice. Centrifuge the cell suspension at 3000 g for 5 min at 4° C.
Discard the supernatant solution. Resuspend the cells in 1 ml of ice-cold 20 mM CaCl₂ solution. Dispense 0.1-ml aliquots into pre-chilled Eppendorf test tubes.
Add about 1 μg of plasmid DNA to the cells.
Freeze the cells in liquid nitrogen.
Thaw the cells by incubating the test tube in a 37° C. water bah for 5 min.
Add 1 ml of YEB medium to the tube and incubate at 28° C. for 2-4 h with gentle shaking.
Centrifuge the tubes for 30s in an Eppendorf centrifuge. Discard the supernatant solution. Resuspend the cells in 0.1 ml YEB medium.
Spread the cells on an YEB agar plate containing Kanamycin at 50 mg/l (because all vectors were pCAMBIA2300-based). Incubate the plate at 28° C. Transformed colonies appeared in 2-3 days.

The integrity of the vectors in Agrobacterium was verified by preparing DNA from Agrobacterium immediately prior to plant transformation.

Production of Transgenic Tobacco Plants

Tobacco leaf discs were used in transformation as described by Rosahl et al. (1987). Transformed plants were selected on MS medium (Murashige and Scoog, 1962) containing cefotaxime (500 mg/l) and kanamycin (100 mg/l). In experiments on evaluation of the effect of different promoters and NLS on expression of tet Receptor, the double transformation technique was employed. In these experiments, tobacco wild type was initially transformed with pAC499 (GUS and HPH genes) and transgenic plants were selected by resistance to hygromycin, 30 mg/l. One of the plants showing high expression of GUS gene was used for the second round of transformation, with vectors carrying tet Receptor and NPTII marker genes.

Plants were maintained in axenic culture on MS basal medium, sucrose (3%), cefotaxime (100 mg/l) with kanamycin (100 mg/l) or hygromycin (30 mg/l).

Production of transgenic Arabidopsis thaliana plants. In planta transformation of Arabidopsis was performed accordingly to the following protocol.
Arabidopsis seeds were sown in lightweight plastic pots with Metro mix covered with window mesh. Plants were grown at 20° C., 16 h. light/18° C., 8 h. dark and became ready for infiltration when the primary inflorescences were 10-15 cm tall.
1 ml of an overnight Agrobacterium culture was inoculated into 100 ml YEB medium containing Kanamycin at 50 mg/l; the culture grew two days at 28° C. and 200 rpm.
When OD₆₀₀ was greater than 2.0, the culture was centrifuged at 3500 rpm for 30 min and resuspended in 100 ml of infiltration medium (Murasige and Scoog salts supplemented with 100 mg/l inositol, 1 mg/l thiamine-HCl, 50 g/l sucrose, 500 mg/l MES, 44 μg/l BAP, pH 5.7 adjusted with KOH before autoclaving and 200 μl/l Silwet L-77 (Osi Specialties, 39 Old Ridgebury Rd., Danbury, Conn. 06810-5121) added before use).
Resuspended culture was placed in a beaker with a large bell jar, and pots with plants were inverted into the solution so that the entire plant was covered. Vacuum of ca. 700 mm Hg was drawn and plants were allowed to sit under the vacuum for 5 min. Pressure was released quickly and plants were drained.
Plants were kept covered for two days and grown under normal conditions from then on. When plants finished flowering (ca. 3 weeks) they dried out for another week and harvested.
Seeds were sterilized and screened on MS medium containing Kanamycin 100 mg/l.Dark green (resistant) plants were transferred directly to soil two weeks after germination. New transplants were kept covered for several days.

GUS histology and fluorescence. For in-vivo staining, intact plant material was vacuum infiltrated with 1 mM x-Gluc (5-bromo-4-chloro-3-indolyl-β-d-glucuronic acid cyclohexylammonium) and incubated overnight at 37° C. For quantitative detection of glucuronidase, GUS assays were performed according to Jefferson (1987) using Wallac Victor ² 1420 Multilabel Counter calibrated with 4-methylumbelliferrone, sodium salt (Sigma). The only modification to the existing protocol was substitution of extraction buffer with Passive Lysis Buffer (Promega Corporation, 2800 Woods Hollow Road, Madison, Wis. 53711-5399, Cat # E1810). The results of fluorescent assays, expressed in μmol/ml, were adjusted by protein concentration in samples, which was measured using Comassie® Plus Protein Assay Reagent (Pierce, P.O. 117, Rockford, Ill. 61105, Cat # 1856210).

Luminescence Analysis.

Dual-Luciferase Reporter Assay System (Promega Corporation, 2800 Woods Hollow Road, Madison, Wis. 53711-5399, Cat # E1810) was used to detect levels of Firefly and, in transient assays, Renilla luciferases in samples. Plant tissue samples were ground in Passive Lysis Buffer and centrifuged at 14000 rpm for 2-3 min. Protoplasts were pelleted (1,000 rpm, 5 min), resuspended in Passive Lysis Buffer, sonicated for 10 min by Branson Sonifier 250 and centrifuged at 3700 rpm and +40° C. for 15 min. Twenty μl of supernatant was used for the assay which was performed accordingly to the protocol supplemented with the kit using Wallac Victor² 1420 Multilabel Counter. The results of luminescent assays, expressed in counts/sec, were adjusted by protein concentration in samples, which was measured using Comassie® Plus Protein Assay Reagent (Pierce, P.O. 117, Rockford, Ill. 61105, Cat # 1856210), and, for transient assays, by the level of Renilla. Somewhat different standardizing system was applied in transient assays. Dual reporter vectors allowed collecting two readings from each sample, Firefly and Renilla. Results for expression of a reporter of interest were adjusted by standard expression of the second reporter: for example, Firefly reading was divided by Renilla reading and multiplied by a large number.

Imaging Plants. The expression of the Firefly luciferase was visualized by low-light video-image analysis. Transgenic plants were sprayed evenly with solution containing 1 mM luciferin (BioSynth International, Inc., 1665 West Quincy Ave., Suite 155, Naperville, Ill. 60540, USA) and 0.1% of Triton-X100followed by immediate measurement of light emission on Night Owl LB 981 (EG&G Berthold, Calmbacher Str. 22, D-75323 Bad Wildbad, Germany)

Obtaining and analysis of progeny. Plants were transferred to pots with Metro soil mix and cultivated in green house under 16-hour daylight period and 26° C. (tobacco) or in growth chambers under similar light conditions and 20° C. (Arabidopsis). After self-pollination seeds were collected and used for both in vitro studies and screening for homozygous plants. The latter was performed as follows. At least six seedlings per line that turned into normal plants under selective pressure after germination on MS plates with appropriate antibiotics were transferred to soil and self-pollinated. T2 seeds were germinated on MS plates and at least sixteen seven-day old seedlings were transferred to MS plates with appropriate antibiotics. All seedlings of homozygous lines were able to grow under selective pressure, whereas segregating lines had antibiotic-susceptible plants.

### Example 3

### Methods for Evaluating the Cassettes of the Invention After Induction with Tetracycline And its Analogs

Evaluation of tet inducible system in transient assays. Protoplasts were co-electroporated with two plasmids, one of which carried an effector gene while the other—dual reporter (Firefly and Renilla luciferases). One of these reporters, usually Firefly luciferase, was under control of an inducible promoter. After electroporation the protoplast aliquot was split between two Petri dishes, one of which was supplemented with a tet analog at 2-5 mg/l. Luminescence assays were performed after 24 hours of cultivation. Dual reporter vectors allowed collecting two readings from each sample, Firefly and Renilla. Results for expression of a reporter of interest were adjusted by standard expression of the second reporter: Firefly reading was divided by Renilla reading and multiplied by a large number.

Evaluation of stably expressing tet inducible system in plant protoplasts. Mesophyll protoplasts from leaves of tobacco plants expressing wild type tet Receptor and tet inducible GUS gene were cultivated at a concentration of 5×10⁴ cells/ml in Kao and Michayluk's 8p medium (Kao, 1975) supplemented with tet analogs at 2-5 mg/l. Protoplasts were cultivated in the dark at 26° C. First divisions were usually observed after 36-60 hours of cultivation; second and sometimes third divisions were visible beginning from the fifth day. Total and divided protoplasts were counted and GUS fluorescent assays performed on the seventh day. In all experiments protoplasts were counted in a fixed volume of 2 mm³ using hemocytometer (Hausser Scientific Partnership). Toxicity of chemicals was evaluated by division rate—the number of divisions divided by the total number of viable cells and multiplied by 100. For quantitative detection of β-glucuronidase, protoplasts were pelleted (1,500 rpm, 5 min), resuspended in extraction buffer (Jefferson, 1987), sonicated for 10 min in a sonication bath and centrifuged. 100 μl of supernatant was used for the assay. GUS assays were performed according to Jefferson (1987) using a Perkin-Elmer fluorimeter calibrated with 4-methylumbelliferrone, sodium salt (Sigma). Assuming that all samples came from a single isolation and contained fixed number of cells, there was no need to adjust results of fluorescent assay with level of protein in protoplasts.

Evaluation of tet inducible system in plant tissues. 5-10 mm leaf disks were excised from plants and put into six- or twelve-well plates with liquid MS medium and with or without a tet analog at 5 mg/l. When transgenic plants carried cassettes for induction of herbicide resistance, the medium was supplemented with PURSUIT® at concentration of 1 μM or higher. These plates were cultivated in dark on shaker at 90 rpm for 5 days (three weeks for analysis of induction of herbicide resistance) and then used in assays or evaluated visually. Another test was designed for evaluation of inducible herbicide resistance. In this test leaf disks were placed on MS agar supplemented with. 1 mg/l of BAP and PURSUIT® at concentration of 1 μM or higher either alone or with a tet analog at 5 mg/l. Three weeks later the induction of regeneration from these disks was evaluated.

Similar test system was applied to root and meristem tissues.

Evaluation of tet inducible system in seed germination test. In this test seeds were placed on MS agar supplemented with or without a tet analog at 5 mg/l. Seven-ten days later, when cotyledons reached 3-5 mm, seedlings were collected and used in assays. For evaluation of inducible herbicide resistance, seeds were placed on MS agar supplemented with PURSUIT® at concentration of 1 μM or higher either alone or with a tet analog at 5 mg/l. The phenotypic effects have been evaluated for two-three weeks. Occasionally, the activation of AHAS gene was evaluated through in vitro test of single plant cultivation in Magenta box on the medium with appropriate chemicals. Plants were evaluated each week during one month.

### Example 4

### Evaluation of Novel 35S-Based Modified Tet-Inducible Promoters

### Analysis of 35S-Based Modified tet Promoters in Plant Protoplasts

The novel tet-inducible promoters engineered on the basis of 35S promoter and placed upstream of luciferase gene in expression vector were evaluated in transient assays after co-electroporation of NT1 protoplasts.

NT1 protoplasts were obtained as follows. Nicotiana tabacum cell line NT1 was obtained from G. An (Washington State University, Pullman). Suspension cultures were grown in a medium containing Murasige and Scoog salts (Gibco Laboratories, Grand Island, N.Y.) supplemented with 100 mg/l inositol, 1 mg/l thiamine-HCl, 180 mg/l KH₂PO_{4, 30} g/l sucrose, and 2 mg/L 2,4-D. The pH was adjusted to 5.7 before autoclaving. Cells were subcultured once a week by adding 2 ml of inoculum to 50 ml of fresh medium in 250-ml Erlenmeyer flasks. The flasks were placed on a rotary shaker at 125 rpm and 27° C. with a light intensity of 47 μmol m⁻²sec⁻¹.

Protoplasts from NT1 cells were isolated accordingly to protocol in Hall, 1991.

Electroporation of NT1 protoplasts was performed as follows. Exponential decay pulses were generated by a Gene Pulser apparatus (Bio-Rad Laboratories, Richmond, Calif.) set at 960 μF and 0.45 kV; 0.4 cm electrode gap potter-type cuvettes (Bio-Rad) were used. An aliquot containing 2.5×10⁶ cells was electroporated accordingly to protocol described by Jones et al (1989) with 20 μg of each of the two plasmids, one of which was the vector under study while the other was pACAG015 carrying NLS-tet Repressor. After electroporation the protoplast aliquot was split between two Petri dishes, one of which was supplemented with doxycycline at 5 mg/l. Cells were resuspended in NT1 medium supplemented with mannitol 0.4M and cultivated on the shaker at 90 rpm and 27° C. with a light intensity of 47 μmol m⁻²sec⁻¹.

Luminescence assays were performed after 24 hours of cultivation. Dual-Luciferase™ Reporter Assay System (Promega Corporation, 2800 Woods Hollow Road, Madison, Wis. 53711-5399, Cat # E1810) was used to detect levels of Firefly luciferase in samples. Protoplasts were pelleted (1,000 rpm, 5 min), resuspended in Passive Lysis Buffer, sonicated for 10 min by Branson Sonifier 250 and centrifuged at 3700 rpm and +4° C. for 15 min. Twenty μl of supernatant were used for the assay which was performed accordingly to the protocol supplemented with the kit using Wallac Victor ² 1420 Multilabel Counter. The results of luminescent assays, expressed in counts/sec, were adjusted by protein concentration in samples, which was measured using Comassie® Plus Protein Assay Reagent (Pierce, P.O. 117, Rockford, Ill. 61105, Cat # 1856210). The results of the luminescent assays from transformed plant protoplasts are shown in FIG. 32 and described elsewhere herein.

### Analysis of 35S-Based Modified Tet Promoters in Plants

Cassettes carrying the novel tet-inducible promoters engineered on the basis of 35S promoter driving Luciferase coding region were cloned into Agrobacterium vector. The resulting plasmids were introduced into Agrobacterium EHA105 strain via the following protocol:
Add 2 ml of overnight Agrobacterium culture to 50 ml YEB medium and shake at 250 rpm and 28° C. until the culture growth to an OD600 of 0.5 to 1.0.
Chill the culture on ice. Centrifuge the cell suspension at 3000 g for 5 min at 4° C.
Discard the supernatant solution. Resuspend the cells in 1 ml of ice-cold 20 mM CaCl₂ solution. Dispense 0.1-ml aliquots into pre-chilled Eppendorf test tubes.
Add about 1 μg of plasmid DNA to the cells.
Freeze the cells in liquid nitrogen.
Thaw the cells by incubating the test tube in a 37° C. water bath for 5 min.
Add 1 ml of YEB medium to the tube and incubate at 28° C. for 2-4 h with gentle shaking.
Centrifuge the tubes for 30 s in an Eppendorf centrifuge. Discard the supernatant solution. Resuspend the cells in 0.1 ml YEB medium.
Spread the cells on an YEB-agar plate containing Kanamycin at 50 mg/l. Incubate the plate at 28° C. Transformed colonies appeared in 2-3 days.

The integrity of the vectors in Agrobacterium was verified by preparing DNA from Agrobacterium immediately prior to plant transformation.

Transgenic tobacco plants were produced as follows. Tobacco (Wisconsin 38) leaf discs were used in transformation as described by Rosahl et al. (1987). Transformed plants were selected on MS medium (Murashige and Scoog, 1962) containing cefotaxime (500 mg/l) and kanamycin (100 mg/l). Plants were maintained in axenic culture on MS basal medium, sucrose (3%), cefotaxime (100 mg/l) with kanamycin (100 mg/l).

The transformation produced at least 10kanamycin-resistant lines per each cassette that were tested in leaf disc induction assay. Ten lines per each cassette were analyzed. One disc from each line was incubated in liquid MS basal medium either supplemented with or without 5 mg/l of doxycycline for 5 days. Disks were collected and used for luciferase assay.

Dual-Luciferase™ Reporter Assay System (Promega Corporation, 2800 Woods Hollow Road, Madison, Wis. 53711-5399, Cat # E1810) was used to detect levels of Firefly luciferase in samples. Leaf discs were ground in Passive Lysis Buffer and centrifuged at 14000 rpm for 2-3 min. Protoplasts were pelleted (1,000 rpm, 5 min), resuspended in Passive Lysis Buffer, sonicated for 10 min by Branson Sonifier 250 and centrifuged at 3700 rpm and +4° C. for 15 min. Twenty μl of supernatant were used for the assay which was performed accordingly to the protocol supplemented with the kit using Wallac Victor² 1420 Multilabel Counter. The results of luminescent assays, expressed in counts/sec, were adjusted by protein concentration in samples, which was measured using

Comassie® Plus Protein Assay Reagent (Pierce, P.O. 117, Rockford, Ill. 61105, Cat # 1856210). Results of the luminescent assays from transgenic plants are presented in FIG. 33 and described elsewhere herein.

### Example 5

### Evaluation of Modified Tet-Inducible Promoters Based on the MAS Promoter

The knowledge gained through reengineering the modified tet-inducible 35S promoters was applied to building the tet induction capability into the (OCS)₃MAS promoter. Four tet-inducible (OCS)₃MAS promoters were constructed and tried in transient assays with NT1 protoplasts.

NT1 protoplasts were obtained as follows. Nicotiana tabacum cell line NT1 was obtained from G. An (Washington State University, Pullman). Suspension cultures were grown in a medium containing Murasige and Scoog salts (Gibco Laboratories, Grand Island, N.Y.) supplemented with 100 mg/l inositol, 1 mg/l thiamine-HCl, 180 mg/l KH₂PO₄, 30 g/l sucrose, and 2 mg/L 2,4-D. The pH was adjusted to 5.7 before autoclaving. Cells were subcultured once a week by adding 2 ml of inoculum to 50 ml of fresh medium in 250-ml Erlenmeyer flasks. The flasks were placed on a rotary shaker at 125 rpm and 27° C. with a light intensity of 47 μmol m ²sec⁻¹. Protoplasts from NT1 cells were isolated accordingly to protocol in Hall, 1991.

Electroporation of NT1 protoplasts was performed as follows. Exponential decay pulses were generated by a Gene Pulser apparatus (Bio-Rad Laboratories, Richmond, Calif.) set at 960 μF and 0.45 kV; 0.4 cm electrode gap potter-type cuvettes (Bio-Rad) were used. An aliquot containing 2.5×10⁶ cells was electroporated accordingly to protocol described by Jones et al (1989) with 20 μg of each of the two plasmids, one of which was the vector under study while the other was pACAG015 carrying NLS-tet Repressor. After electroporation the protoplast aliquot was split between two Petri dishes, one of which was supplemented with doxycycline at 5 mg/l. Cells were resuspended in NT1 medium supplemented with mannitol 0.4M and cultivated on the shaker at 90 rpm and 27° C. with a light intensity of 47 μmol. m⁻²sec⁻¹.

Luminescence assays were performed after 24 hours of cultivation. Dual-Luciferase™ Reporter Assay System (Promega Corporation, 2800 Woods Hollow Road, Madison, Wis. 53711-5399, Cat # E1810) was used to detect levels of Firefly luciferase in samples. Protoplasts were pelleted (1,000 rpm, 5 min), resuspended in Passive Lysis Buffer, sonicated for 10 min by Branson Sonifier 250 and centrifuged at 3700 rpm and +40° C. for 15 min. Twenty μl of supernatant were used for the assay which was performed accordingly to the protocol supplemented with the kit using Wallac Victor² 1420 Multilabel Counter. The results of luminescent assays, expressed in counts/sec, were adjusted by protein concentration in samples, which was measured using Comassie® Plus Protein Assay Reagent (Pierce, P.O. 117, Rockford, Ill. 61105, Cat # 1856210). The results of the lumenescent assays are shown in FIG. 34.

### Example 6

### Evaluation of Cassette Orientation Effects on the Modified Tet-Inducible Promoters

Cassette orientation effects of the modified tet-inducible promoters of the present invention were evaluated in a new set of transgenic Arabidopsis plants carrying pACAG119, pACAG119r, pACAG120 and pACAG120r (all carrying (OCS)₃TripleXₘ/AHAS, Actin-intron/nTR and NPTII cassettes in different layouts).

These vectors-were introduced into Agrobacterium EHA105 strain via the following protocol:
Add 2 ml of overnight Agrobacterium culture to 50 ml YEB medium and shake at 250 rpm and 28° C. until the culture growth to an OD600 of 0.5 to 1.0.
Chill the culture on ice. Centrifuge the cell suspension at 3000 g for 5 min at 4° C.
Discard the supernatant solution. Resuspend the cells in 1 ml of ice-cold 20 mM CaCl₂ solution. Dispense 0.1 ml aliquots into pre-chilled Eppendorf test tubes.
Add about 1 μg of plasmid DNA to the cells.
Freeze the cells in liquid nitrogen.
Thaw the cells by incubating the test tube in a 37° C. water bah for 5 min.
Add 1 ml of YEB medium to the tube and incubate at 28° C. for 2-4 h with gentle shaking.
Centrifuge the tubes for 30 s in an Eppendorf centrifuge. Discard the supernatant solution. Resuspend the cells in 0.1 ml YEB medium.
Spread the cells on an YEB agar plate containing Kanamycin at 50 mg/l. Incubate the plate at 28° C. Transformed colonies appeared in 2-3 days.

The integrity of the vectors in Agrobacterium was verified by preparing DNA from Agrobacterium immediately prior to plant transformation.

Transgenic Arabidopsis thaliana plants were produced accordingly to the following protocol:
Arabidopsis seeds (Columbia) were sown in lightweight plastic pots with Metro mix covered with window mesh. Plants were grown at 20° C., 16 h light/18° C., 8 h dark and became ready for infiltration when the primary inflorescences were 10-15 cm tall.
1 ml of an overnight Agrobacterium culture was inoculated into 100 ml YEB medium containing Kanamycin at 50 mg/l; the culture grew two days at 28° C. and 200 rpm.
When OD₆₀₀ was greater than 2.0, the culture was centrifuged at 3500 rpm for 30 min and resuspended in 100 ml of infiltration medium (Murasige and Scoog salts supplemented with 100 mg/l inositol, 1 mg/l thiamine-HCl, 50 g/l sucrose, 500 mg/l M:S, 44 μg/l BAP, pH 5.7 adjusted with KOH before autoclaving and 200 μl/l Silwet L-77 (Osi Specialties, 39 Old Ridgebury Rd., Danbury, Conn. 06810-5121) added before use).
Resuspended culture was placed in a beaker with a large bell jar, and pots with plants were inverted into the solution so that the entire plant was covered. Vacuum of ca. 700 mm Hg was drawn and plants were allowed to sit under the vacuum for 5 min. Pressure was released quickly and plants were drained.
Plants were kept covered for two days and grown under normal conditions from then on. When plants finished flowering (ca. 3 weeks) they dried out for another week, harvested, and vernalized at +40° C. for 7 days.
Seeds were sterilized and screened on MS medium containing Kanamycin 100 mg/l. Dark green (resistant) plants were transferred directly to soil two weeks after germination. New transplants were kept covered for several days.
It took ca. 6 weeks for these plants to set seeds. Plants were dried out for two additional days after which seeds were collected and vernalized at +40° C. for 7 days.

Transgenic seeds were germinated on the MS basal media with sucrose 3% and either PURSUIT® 1 μM alone or PURSUIT® 1 μM and doxycycline 5 mg/l. Results were evaluated two weeks later. Even though between 6 and 14 lines were tested per cassette, results were highly consistent for lines representing the same cassette. Therefore it was possible to identify typical response patterns of plants upon addition of doxy. The results are presented in FIG. 35 and are described elsewhere herein.

### Example 7

### Spray Test for Induction of Herbicide Resistance

Induction of herbicide resistance in plants growing in soil has been investigated. Transgenic F1 heterozygous tobacco seeds of the line pACAG029#4 (this line has been shown elsewhere herein to provide the best repression/induction in tissue culture tests) ere used for the spray test. Seedlings at different stages of development (1 and 2 weeks old) produced by germination of seeds in 2.5″×2.5″ pots with Metro mix were used for post-emergence test. For pre-emergency application, seeds were sown on the Metro mix right before the application of herbicide. 15-20 seeds were placed in each pot. Pots were sprayed with doxycycline premixed with PURSUIT®, both at different rates. Rates for PURSUIT® were chosen based on commercially recommended rates (62 g/ha as 1 fold (1×), for reference see Herbicide handbook. Weed Science Society of America, 7^{th} edition, 1994, which is hereby incorporated herein by reference); rates for doxycycline were chosen arbitrarily. Spraying mixtures were acetone-based (active ingredients were dissolved in straight acetone and made up to the final concentration with water so that the final concentration of acetone in the mix was 5%). In addition to the active ingredients, PURSUIT® and doxycycline, the spray mixtures contained the surfactant, Sun It II (methylated seed oil), at 0.5% final volume. Spraying was performed in a custom-made sprayer. Spray volume was 300 L/ha. After application, pots were incubated without watering for 48 hours, after which they were watered regularly. Pots were kept at 28° C., 16 h light/25° C., 8 h dark in the greenhouse.

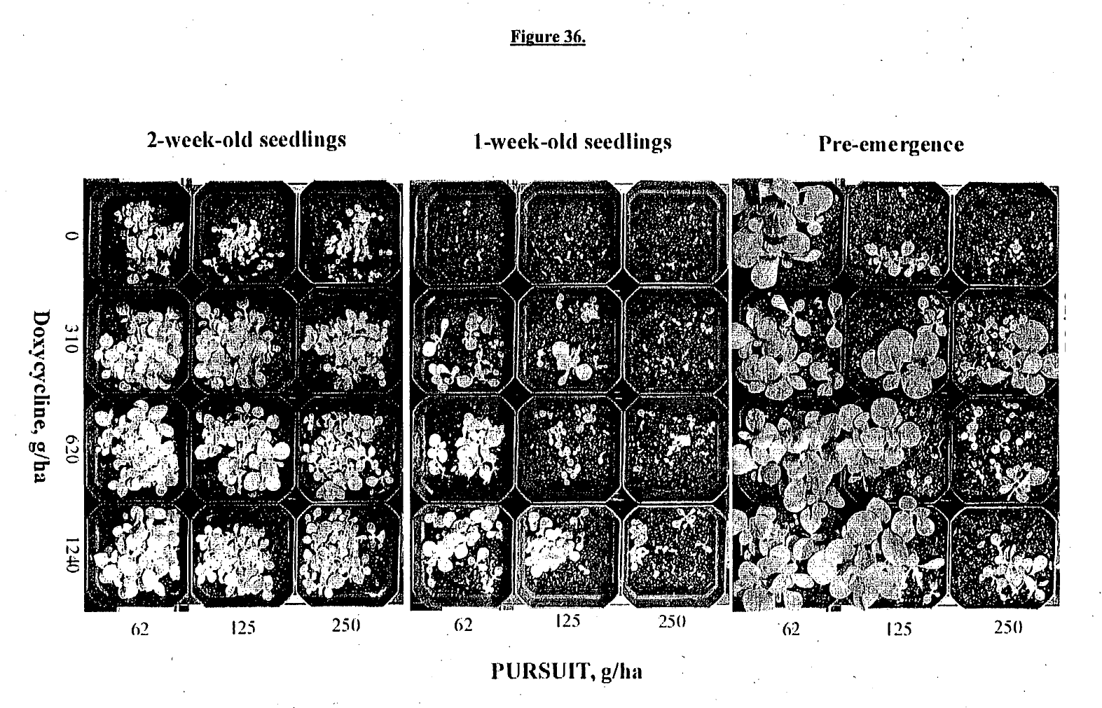

The results of the test was evaluated two weeks after spray. Results are shown in FIG. 36. Results proved that herbicide resistance could be turned on via chemical switch in the field conditions either pre- or post-emergence. Among different herbicide rates, 250 g/ha (four fold rate) of PURSUIT® seemed to be too strong even for induced plants, whereas 62 g/ha (1 fold) was too weak to see the difference between induced and uninduced seedlings. Therefore, the 2 rate could be the dose of choice for the field applications. For doxycycline, both 310 and 620 g/ha were the rates that effectively induced resistance to PURSUIT®, whereas 1.24 kg/ha premixed with the 2 fold and 4 fold rates of herbicide caused some injury and stunted growth of plants. These finding were consistent across the range of stages of plant development. To summarize, the following combinations could be effectively used to turn herbicide resistance on using the modified tet inducible system of the present invention in tobacco growing in soil either pre- or post-emergence (pACAG029#4):
Doxycycline, g/ha 310 or 620
PURSUIT®, g/ha 125

### Example 8

### High-Throughput Method for Identifying Novel Tetracycline Analogs and/or Functional Equivalents Using Modified Tetracycline-Inducible Promoter Cassettes of the Present Invention

The present invention encompasses the application of the modified tetracycline-inducible promoter cassettes to the identification of novel tetracycline analogs and/or functional equivalents in a high-throughput system. Specifically, the invention encompasses a method for high throughput screening of chemical compounds using an agar based plant growth system to identify tetracycline analogs and/or functional equivalents which can induce herbicide resistance in Arabidopsis. For example, such a high-throughput method may preferably comprise the AHAS gene under the inducible control of a modified tetracycline-inducible promoter cassette of the present invention (pACAG029, described elsewhere herein).

Other herbicide conferring resistance genes that could be substituted for the AHAS gene in this system are known in the art and are referenced elsewhere herein. Moreover, such a system could be applied to other species using methods known in the art. Such species are known in the art are referenced elsewhere herein.

Sample Preparation

Aliquots of test compounds were dissolved in acetone and placed in a 96 well test plate for use in the high throughput screen. The acetone solution was allowed to evaporate from the test plate for at least four hours.

Agar Preparation

Murashige and Skoog Minimal Organics (MMOM) media was prepared by weighing 4.6 g of MMOM powder (Gibco Life Technologies) and 7 g of Phytagar (Gibco Life Technologies) into a two liter autoclave bottle. 1 μM imazethapyr in one liter of distilled water was added to the bottle. The resulting mixture was heated to boiling until the media was clear. A second liter of media was prepared without imazethapyr.

300 μl of hot MMOM media with herbicide was dispensed into 88 wells of the test plates. The hot MMOM media without herbicide was added to the remaining eight wells and served as control wells. The test plates were allowed to cool for at least two hours.

### Test Preparation, Growth and Evaluation

Transgenic Arabidopsis seeds were placed on the surface of the agar in all of the wells of the test plates. The test plates were placed under 24 hour fluorescent lights in a growth chamber set at 22° C. After 4-5 days an evaluation of the plates was made.

Three observations for each plate were determined:
a) The seeds in the wells containing no herbicide in the agar and no test compound should germinate and grow.
b) The seeds in the wells containing herbicide in the agar and no test compounds should not grow.
c) The seeds in wells containing herbicide in the agar and test compounds should not grow unless a test compound induced the expression of the herbicide resistance gene (e.g., AHAS, etc.) under the control of a modified tetracycline-inducible promoter cassette of the invention in Arabidopsis. Based upon the herbicide resistance, the plants contained within this well should grow and look similar to the control wells provided as observation “a” above.

Alternatively, seeds in wells containing herbicide plus an inducer of the tet-repressor (e.g., tetracycline, doxycyline, etc.) will show germination and growth similar to seeds in control wells that contain no herbicide.

The herbicide that may be applied to the high-throughput method outlined above may be herbicides belonging to the imazethapyr family for which the AHAS gene is known to confer resistance, which include the following, none limiting examples: imazamethabenz, imazapyr, imazaquin, etc. In addition, the method also encompasses the application of the following, non-limiting, examples of herbicides for which the AHAS gene may also confer resistance: sulfonylurea herbicides, bensulfuron, CGA-152005, chlorimuron, chlorsulfuron, ethametsulfuron, metsulfuron, mon 12000, nicosulfuron, primisulfuron, sulfometuron, thifensulfuron, triasulfuron, tribenuron, and triflusulfuron, for example.

The method could be modified to utilize other high-throughput formats aside from 96 well test plates, such as 384 well test plates, and perhaps 1536 well test plates, for example. Such modifications would be appreciated by the skilled artisan and are encompassed by the present invention.

### Example 9

### Antibody Mediated Down-Regulation of Plant Proteins

The process of genetically modifying a plant to modulate specific characteristics, to introduce novel traits, or to inhibit endogenous traits represents a significant area of research in the agricultural field. Recently, a new method of modulating endogenous gene expression using antibodies has been elucidated (see, International Publication Number WO 00/05391, which is hereby incorporated in its entirety herein). In this example, the researchers were able to achieve 40-70% inhibition of an endogenous plant protein through the use of a single-chain antibody gene directed towards the plant protein.

The method is directed towards the production of monoclonal antibodies, specifically, single chain antibodies, specific to endogenous transit peptides in a plant in an effort to decrease steady state levels of such transit peptides within the plant. The method is comprised of the following steps: I) generating monoclonal antibodies to a specific plant, II) cloning the gene for said monoclonal antibody, III) creating an expression vector comprising a fusion of the heavy-chain and light chain gene sequences of said monoclonal antibody gene downstream of the p67 leader peptide, and under the control of a constitutive plant promoter, IV) optimizing the codons of said heavy-chain and light chain fusion vector for efficient expression of the gene encoded thereof in a plant, and V), transfecting a plant with said heavy-chain and light chain fusion expression vector.

The vector containing the antibody gene could easily be modified to comprise a modified tetracycline inducible repressor, operator, and/or repressor/operator cassette of the present invention.

The skilled artisan would appreciate the methods described therein (WO 00/05391), and would have the ability to apply such methods to inhibiting the steady-state expression levels of any of the polypeptides of the present invention, including variants, and fragments, thereof. The skilled artisan would appreciate that any leader peptide (i.e., signal sequence) from a plant protein could be used in creating the heavy-chain and light chain fusion vector. The skilled artisan would also appreciate that different plant species may have different codon usage requirements, and thus, the decision to optimize the codons of the heavy-chain and light chain fusion vector would be affected according to the codons required for that particular plant species.

The method could not only be applied to transit peptides, but also to secreted proteins, membrane proteins, receptors, and ligands. The method could also be applied in combination with other antibody production methods in plants. For example, antibodies directed towards polypeptides of the present invention may inhibit specific traits in a plant which could increase the plants defense mechanisms to pathogens. Thus, where such an antibody was expressed, another antibody could be expressed in combination with the first, to inhibiting the pathogenicity of a plant pathogen by directing the expression of antibodies directed towards pathogenic proteins (e.g., those proteins critical to the initiating events of infection, such as the BUF1 gene from M. grisea, stage two juvenile salivary gland proteins which include, svp30, scp31a, scp31b, scp32, scp32, scp39, and scp49 from G. rostochiensis (WO 96/22372), etc.). Such a combination would also be of value where the second “anti-pathogenic” antibody is an antibody directed towards a pathogen and fused to a toxic protein wherein such a toxin could be chitinase, glucanase, lysozyme, BT, or colicin F, for example (see WO 96/09398), etc.).

The method could also be used as a means of inhibiting allergic reactions to plant antigens in humans, mammals, animals, etc., by directing the production of a single chain antibody specific towards said plant antigen in the plant (via transgenic methodology). In the latter example, the plant would not be limited to edible plants, as inhibiting the production of such a plant antigen would provide benefit to a human by removing the antigen from the humans environment, for example, irrespective of whether the plant was ingested.

Of particular interest to this example, is the fact that secretion of functional antibody through the plasma membrane of plant cells has been reported for protoplasts isolated from transgenic plants and for callus cells adapted to suspension culture (Hein et al., Biotechnol. Prog. 7:455-561, 1991). However, the levels of secreted antibody detected in both culture systems were extremely low. In other studies, cultured tobacco cells were transformed with a gene encoding a synthetic antibody derivative expressed as a single chain consisting of both the heavy- and light-chain variable domains of the intact immunoglobulin joined together by a flexible peptide linker (Pluckthun, Immunol. Rev. 130:151-188, 1991; and Bird et al., Science 242:423-426, 1988). This synthetic single-chain antibody retained the full antigen-binding potential of the intact immunoglobulin but accumulated in the extracellular apoplastic space of the transformed cells (Firek et al., Plant Molecular Biology 23:861-870, 1993), indicating that the antibody was being transported through the plasma membrane but not through the cell wall to the external environment. Moreover, recent studies have shown that increased antibody production in a plant, and heterologous protein expression, in general, could be increased by including in the plant culture medium a protein stabilizing agent (e.g., polyvinylpyrrolidone), see U.S. Pat. No. 6,020,169, which is hereby incorporated by reference in its entirety herein.

1. A novel tetracycline repressor cassette comprising a promoter operably linked to the tetracycline repressor coding sequence, a transcriptional terminator sequence, a first enhancer sequence, and optionally a second enhancer sequence.

2. The novel tetracycline repressor cassette of claim 1 further comprising a nuclear localization signal.

3. The novel tetracycline repressor cassette of claim 2 wherein the promoter is selected from the group consisting of: a mannopine synthase promoter (MAS), a minimal MAS promoter, a Arabidopsis thaliana acetohydroxyacid synthase promoter (AtAHAS), a Arabidopsis thaliana hydroxyphenylpyruvate dioxygenase promoter (AtHPPD), a Arabidopsis thaliana Actin-Intron promoter, a Cauliflower Mosaic Virus 35s promoter, a two tandem CMV promoters—2×35s and a 35s minimal promoter.

4. The novel tetracycline repressor cassette of claim 2 wherein the promoter is operable in plants.

5. The novel tetracycline repressor cassette of claim 2 wherein the promoter is operable in animals.

6. The novel tetracycline repressor cassette of claim 4 wherein the transcriptional terminator sequence is selected from the group consisting of: an octopine synthase terminator sequence, and a NOS terminator sequence.

7. The novel tetracycline repressor cassette of claim 6 wherein the first enhancer is selected from the group consisting of: a single octopine synthase activating sequence, two octopine synthase activating sequences, a triad of octopine synthase activating sequences (i.e., (OCS)₃), and a single matrix attachment region sequence (MAR).

8. The novel tetracycline repressor cassette of claim 7 wherein the first enhancer is located 5′ of the promoter.

9. The novel tetracycline repressor cassette of claim 7 wherein the first enhancer is located 3′ of the promoter.

10. The novel tetracycline repressor cassette of claims 8 or 9 wherein the second enhancer is selected from the group consisting of: a single octopine synthase activating sequence, two octopine synthase activating sequences, a triad of octopine synthase activating sequences (i.e., (OCS)₃), and a single matrix attachment region sequence (MAR).

11. The novel tetracycline repressor cassette of claim 10 wherein the second enhancer is located 5′ of the promoter.

12. The novel tetracycline repressor cassette of claim 10 wherein the second enhancer is located 3′ of the promoter.

13. The novel tetracycline repressor cassette of claim 11 or 12 wherein the promoter is transcriptionally oriented in the 5′ direction.

14. The novel tetracycline repressor cassette of claim 11 or 12 wherein the promoter is transcriptionally oriented in the 3′ direction.

15. A novel tetracycline operator cassette comprising a promoter with tetracycline operator sequences, a coding region of a polynucleotide sequence of interest, a transcriptional terminator, a first enhancer sequence, and optionally, a second enhancer sequence.

16. The novel tetracycline operator cassette of claim 15 wherein the promoter with tetracycline operator sequences comprises a member of the group selected from consisting of:
a) one tetracycline operator sequence,
b) two tetracycline operator sequences,
c) three tetracycline operator sequences, or
d) four tetracycline operator sequences.

17. The novel tetracycline operator cassette of claim 16 wherein the promoter with tetracycline operator sequences is selected from the group consisting of: SEQ ID NO:4, and a TripleX promoter.

18. The novel tetracycline operator cassette of claim 17 wherein the promoter is operable in plants.

19. The novel tetracycline operator cassette of claim 17 wherein the promoter is operable in animals.

20. The novel tetracycline operator cassette of claim 18 wherein the polynucleotide sequence of interest is a member selected from the group consisting of: a plant gene, an herbicide resistance gene, a reporter gene, an insecticide resistance gene, a fungicide resistance gene, a viral resistance gene, a cold tolerance gene, a water stress tolerance gene, a stress tolerance gene, an input trait gene, and a output trait gene.

21. The novel tetracycline operator cassette of claim 20 wherein the transcriptional terminator sequence is selected from the group consisting of: an octopine synthase terminator sequence, and a NOS terminator sequence.

22. The novel tetracycline operator cassette of claim 21 wherein the first enhancer is selected from the group consisting of: a single octopine synthase activating sequence, two octopine synthase activating sequences, a triad of octopine synthase activating sequences (i.e., (OCS)₃), and a single matrix attachment region sequence (MAR).

23. The novel tetracycline operator cassette of claim 22 wherein the first enhancer is located 5′ of the promoter.

24. The novel tetracycline operator cassette of claim 22 wherein the first enhancer is located 3′ of the promoter.

25. The novel tetracycline operator cassette of claims 23 or 24 wherein the second enhancer is selected from the group consisting of: a single octopine synthase activating sequence, two octopine synthase activating sequences, a triad of octopine synthase activating sequences (i.e., (OCS)₃), and a single matrix attachment region sequence (MAR).

26. The novel tetracycline operator cassette of claim 25 wherein the second enhancer is located 5′ of the promoter.

27. The novel tetracycline operator cassette of claim 25 wherein the second enhancer is located 3′ of the promoter.

28. The novel tetracycline operator cassette of claim 25 wherein the promoter is transcriptionally oriented in the 5′ direction.

29. The novel tetracycline operator cassette of claim 25 wherein the promoter is transcriptionally oriented in the 3′ direction.

30. A novel tetracycline repressor/operator cassette comprising a first promoter operably linked to the tetracycline repressor coding sequence, a first transcriptional terminator sequence, a first enhancer sequence, and optionally a second enhancer sequence, wherein said first and/or second enhancer sequences are associated with said first promoter, a second promoter with at least one tetracycline operator sequences, a coding region of a polynucleotide sequence of interest, a second transcriptional terminator located 3′ of the polynucleotide sequence of interest, a third enhancer sequence, and optionally, a fourth enhancer sequence, wherein the third and/or fourth enhancer sequences are associated with said second promoter.

31. The novel tetracycline repressor/operator cassette of claim 30 wherein the tetracycline repressor coding sequence further comprises a nuclear localization signal.

32. The novel tetracycline repressor/operator cassette of claim 31 wherein the first promoter is selected from the group consisting of: a mannopine synthase promoter (MAS), a minimal MAS promoter, a Arabidopsis thaliana acetohydroxyacid synthase promoter (AtAHAS), a Arabidopsis thaliana hydroxyphenylpyruvate dioxygenase promoter (AtHPPD), a Arabidopsis thaliana Actin-Intron promoter, a Cauliflower Mosaic Virus 35s promoter, a two tandem CMV promoters—2×35s and a 35s minimal promoter.

33. The novel tetracycline repressor/operator cassette of claim 32 wherein the first promoter is operable in plants.

34. The novel tetracycline repressor/operator cassette of claim 32 wherein the first promoter is operable in animals.

35. The novel tetracycline repressor/operator cassette of claim 33 wherein the transcriptional terminator sequence is selected from the group consisting of: an octopine synthase terminator sequence, and a NOS terminator sequence.

36. The novel tetracycline repressor/operator cassette of claim 35 wherein the first enhancer is selected from the group consisting of: a single octopine synthase activating sequence, two octopine synthase activating sequences, a triad of octopine synthase activating sequences (i.e., (OCS)₃), and a single matrix attachment region sequence (MAR).

37. The novel tetracycline repressor/operator cassette of claim 36 wherein the first enhancer is located 5′ of the promoter.

38. The novel tetracycline repressor/operator cassette of claim 36 wherein the first enhancer is located 3′ of the promoter.

39. The novel tetracycline repressor/operator cassette of claims 37 or 38 wherein the second enhancer is selected from the group consisting of: a single octopine synthase activating sequence, two octopine synthase activating sequences, a triad of octopine synthase activating sequences (i.e., (OCS)₃), and a single matrix attachment region sequence (MAR).

40. The novel tetracycline repressor/operator cassette of claim 39 wherein the second enhancer is located 5′ of the promoter.

41. The novel tetracycline repressor/operator cassette of claim 39 wherein the second enhancer is located 3′ of the promoter.

42. The novel tetracycline repressor/operator cassette of claim 40 or 41 wherein the second enhancer is selected from the group consisting of: a single octopine synthase activating sequence, two octopine synthase activating sequences, a triad of octopine synthase activating sequences (i.e., (OCS)₃), and a single matrix attachment region sequence (MAR).

43. The novel tetracycline repressor/operator cassette of claim 42 wherein the first enhancer, second enhancer, and the first transcriptional terminator are associated with the first promoter and coding region of the tetracycline repressor.

44. The novel tetracycline repressor/operator cassette of claim 42 wherein the second promoter with tetracycline operator sequences comprises a member selected from the group consisting of:
a) one tetracycline operator sequence,
b) two tetracycline operator sequence,
c) three tetracycline operator sequence, or
d) four tetracycline operator sequence.

45. The novel tetracycline repressor/operator cassette of claim 43 wherein the second promoter with tetracycline operator sequences is selected from the group consisting of: SEQ ID NO:4, and a TripleX promoter.

46. The novel tetracycline repressor/operator cassette of claim 45 wherein the second promoter is operable in plants.

47. The novel tetracycline repressor/operator cassette of claim 45 wherein the second promoter is operable in animals.

48. The novel tetracycline repressor/operator cassette of claim 46 wherein the polynucleotide sequence of interest is selected from the group consisting of: genes capable of being expressed in plants, plant genes, selectable markers genes, reporter genes, genes encoding agronomic traits, transcriptional activators, antisense genes, sense genes, cDNAs, genes encoding input traits, genes encoding output traits, or the AtAHAS (S653N) mutant gene.

49. The novel tetracycline repressor/operator cassette of claim 48 wherein the second transcriptional terminator sequence is selected from the group consisting of: an octopine synthase terminator sequence, and a NOS terminator sequence.

50. The novel tetracycline repressor/operator cassette of claim 49 wherein the third enhancer is selected from the group consisting of: a single octopine synthase activating sequence, two octopine synthase activating sequences, a triad of octopine synthase activating sequences (i.e., (OCS)₃), and a single matrix attachment region sequence (MAR).

51. The novel tetracycline repressor/operator cassette of claim 50 wherein the third enhancer is located 5′ of the promoter.

52. The novel tetracycline repressor/operator cassette of claim 50 wherein the third enhancer is located 3′ of the promoter.

53. The novel tetracycline repressor/operator cassette of claims 51 or 52 wherein the fourth enhancer is selected from the group consisting of: a single octopine synthase activating sequence, two octopine synthase activating sequences, a triad of octopine synthase activating sequences (i.e., (OCS)₃), and a single matrix attachment region sequence (MAR).

54. The novel tetracycline repressor/operator cassette of claim 53 wherein the fourth enhancer is located 5′ of the promoter.

55. The novel tetracycline repressor/operator cassette of claim 53 wherein the fourth enhancer is located 3′ of the promoter.

56. The novel tetracycline repressor/operator cassette of claim 54 or 55 wherein the first and second promoters, in addition to their accompanying enhancer, transcriptional terminator, and gene(s) of interest, are transcriptionally oriented in a similar direction.

57. The novel tetracycline repressor/operator cassette of claim 54 or 55 wherein the first and second promoters, in addition to their accompanying enhancer, transcriptional terminator, and gene(s) of interest, are transcriptionally oriented in an opposing direction.

58. A method of modulating the expression of a gene in a plant comprising the following steps:
a) Creating a vector comprising the coding region of a polynucleotide sequence of interest inserted downstream of a member of the group consisting of: the second promoter of the novel tetracycline repressor/operator cassette of claim 56, and second promoter of the novel tetracycline repressor/operator cassette of claim 57,
b) Transfecting the plant with the vector of step ‘a’, and
c) Subjecting said transfected plant to a tetracycline analog and/or functional equivalent.

59. A method of making a plant herbicide resistant comprising the following steps:
a) Creating a vector comprising the coding region of an herbicide resistance gene inserted downstream of a member of the group consisting of: the second promoter of the novel tetracycline repressor/operator cassette of claim 56, and the second promoter of the novel tetracycline repressor/operator cassette of claim 57,
b) Transfecting the plant with the vector of step ‘a’, and
c) Subjecting said transfected plant to a tetracycline analog and/or functional equivalent.

60. The method of making a plant herbicide resistant of claim 59 wherein the polynucleotide sequence of interest is selected from the group consisting of: the polynucleotide sequence encoding the AtAHAS (S653N) mutant gene.

61. A method of expressing a gene of interest in specific plant tissues comprising the following steps:
a) Creating a vector comprising the coding region of a gene of interest inserted downstream of a member of the group consisting of: the second promoter of the novel tetracycline repressor/operator cassette of claim 56, and the second promoter of the novel tetracycline repressor/operator cassette of claim 57,
b) Transfecting the plant with the vector of step ‘a’, and
c) Subjecting said transfected plant to a tetracycline analog and/or functional equivalent.

62. A method of modulating the gene expression of a plant comprising the following steps:
a) Creating monoclonal antibodies directed against the gene of interest,
b) Isolating the coding region of the antibody gene directed against the gene of interest,
c) Creating a vector comprising the coding region of the antibody directed against the gene of interest inserted downstream of a member of the group consisting of: the second promoter of the novel tetracycline repressor/operator cassette of claim 56, the second promoter of the novel tetracycline repressor/operator cassette of claim 57,
d) Transfecting the plant with the vector of step ‘c’, and
e) Subjecting said transfected plant to a tetracycline analog and/or functional equivalent.

63. A method of identifying novel tetracycline analogs and/or functional equivalents
a) Creating a vector comprising the coding region of a gene of interest inserted downstream of the second promoter of a member of the group consisting of: the novel tetracycline repressor/operator cassette of claim 56, and the novel tetracycline repressor/operator cassette of claim 57,
b) Transfecting the plant with the vector of step ‘a’,
c) Subjecting said transfected plant to a chemical compound, and
d) Assessing whether the plant expressed the gene of interest.

64. The method of claim 63 wherein the gene of interest is a member of the group consisting of: genes capable of being expressed in plants, plant genes, selectable markers genes, reporter genes, genes encoding agronomic traits, transcriptional activators, antisense genes, sense genes, cDNAs, genes encoding input traits, genes encoding output traits, or the AtAHAS (S653N) mutant gene.

65. A recombinant vector comprising a member of the group consisting of: novel tetracycline repressor cassette of claim 1, the novel tetracycline repressor cassette of claim 8, novel tetracycline repressor cassette of claim 9, novel tetracycline repressor cassette of claim 10, novel tetracycline repressor cassette of claim 11, novel tetracycline repressor cassette of claim 12, novel tetracycline operator cassette of claim 15, novel tetracycline repressor cassette of claim 23, novel tetracycline repressor cassette of claim 24, novel tetracycline repressor cassette of claim 25, novel tetracycline repressor/operator cassette of claim 30, novel tetracycline repressor/operator cassette of claim 37, novel tetracycline repressor/operator cassette of claim 38, novel tetracycline repressor/operator cassette of claim 39, novel tetracycline repressor/operator cassette of claim 40, novel tetracycline repressor/operator cassette of claim 41, novel tetracycline repressor/operator cassette of claim 42, novel tetracycline repressor/operator cassette of claim 43, novel tetracycline repressor/operator cassette of claim 44, novel tetracycline repressor/operator cassette of claim 45, novel tetracycline repressor/operator cassette of claim 46, novel tetracycline repressor/operator cassette of claim 47, novel tetracycline repressor/operator cassette of claim 48, novel tetracycline repressor/operator cassette of claim 49, novel tetracycline repressor/operator cassette of claim 50, novel tetracycline repressor/operator cassette of claim 51, novel tetracycline repressor/operator cassette of claim 52, novel tetracycline repressor/operator cassette of claim 53, novel tetracycline repressor/operator cassette of claim 54, novel tetracycline repressor/operator cassette of claim 55, novel tetracycline repressor/operator cassette of claim 56, and novel tetracycline repressor/operator cassette of claim 57.

66. A method of making a recombinant host cell comprising the step of transfecting a host cell with the recombinant vector of claim 65.

67. A recombinant host cell produced by the method of claim 66.

68. The recombinant host cell of claim 67 comprising vector sequences.
